(19) United States
(12) Patent Application Publication
(10) Pub. No.: US 2010/0150958 A1
(43) Pub. Date: Jun. 17, 2010

(54) Methods and Compositions for Use of a Coccidiosis Vaccine

(75) Inventor: Michael Sheppard, Eltham (AU)

Correspondence Address: MCANDREWS HELD & MALLOY, LTD, 500 WEST MADISON STREET, SUITE 3400, CHICAGO, IL, 60661, US

(73) Assignee: VECTOGEN PTY LTD., Perth (AU)

(21) Appl. No.: 12/621,421

(22) Filed: Nov. 18, 2009

Related U.S. Application Data
(60) Provisional application No. 61/122,596, filed on Dec. 15, 2008.

(51) Int. Cl. A61K 39/235; A61P 33/00; A61K 39/17; A61K 39/215; A61K 39/295; A61K 39/145; A61K 39/15; A61K 39/21; A61K 39/155; C12N 15/861; C12N 5/00

(52) U.S. Cl. 424/201.1; 424/233.1; 424/229.1; 424/214.1; 424/222.1; 424/202.1; 424/209.1; 424/215.1; 424/207.1; 424/211.1; 435/320.1; 435/325

(57) ABSTRACT

The invention relates to new vaccine compositions for vaccinating birds.

### RELATED APPLICATIONS

The present application claims priority of U.S. Provisional Patent Application No. 61/122,596, which was filed on Dec. 15, 2008. The entire text of the aforementioned application is incorporated herein by reference in its entirety.

### FIELD OF THE INVENTION

The present invention relates to methods and compositions for vaccination of birds.

### BACKGROUND OF THE INVENTION

Coccidiosis is an extremely important disease of chickens worldwide. It results in estimated losses to the broiler industry alone of more than 1 billion USD per year. Coccidiosis is caused by infection with seven species of the apicomplexan protozoan parasite Eimeria. Of these seven species, E. tenella, E. maxima and E. acervuline are considered to be the most problematic. Symptoms of coccidiosis include listlessness, anemia, watery or bloody diarrhea (depending on the infecting species), weight loss and poor feed conversion ratios.

The growth and spread of Eimeria parasites is particularly prevalent in chickens because these birds are typically reared under crowded conditions which make it extremely difficult to maintain sanitary control. The use of coccidiostat drugs under these conditions is ineffective due to the development of resistance and due to difficulties in sustained administration of such drugs in cramped feeding environments. Furthermore, coccidiostats also have antibacterial effects and the use of in-feed antibiotics is deemed undesirable.

The United States Department of Agriculture (USDA) has recognized that the U.S. and worldwide broiler industry relies heavily on the use of anti-coccidial drugs, which are added to poultry feed and prevent the intracellular development of Eimeria stages inside the chicken gut. The drugs are removed from feed about 1 week prior to the chickens being sent to market as a way of preventing drug residues in the meat product. While anti-coccidial drugs continue to be the primary means of preventing avian coccidiosis, the USDA has stated that the ability of Eimeria parasites to become resistant to such drugs requires development of alternative control measures.

One alternative solution to combating coccidiosis would be to develop an effective vaccine. While administration of a mixture of low doses of virulent or attenuated Eimeria species oocysts has been contemplated it remains to be proven as an effective intervention in reality. Because chickens develop immunity to Eimeria, substantial efforts are being expended to develop “subunit” vaccines against coccidiosis. Such vaccines would utilize genetic engineering technology to produce protein components of Eimeria parasites. The rationale behind this approach is that harmless, laboratory strains of bacteria can be utilized to produce “recombinant” proteins that may be used to immunize chickens either in ovo (in the egg) or at hatch. If successful, chickens will be resistant to a subsequent Eimeria infection because they have been immunized with a protein that is normally present on the surface of the parasite. However, according to the USDA, the efforts to date have failed to come to fruition and, as yet, there are no commercial subunit vaccines available to prevent avian coccidiosis.

CoxAbic® is a vaccine gaining some acceptance in the industry and is based on using three major affinity purified, full length native antigens (of 56 kDa, 82 kDa and 230 kDa) isolated from the macrogametocyte (female sexual) stage of development of Eimeria maxima to vaccinate laying hens just prior to the start of their laying period. CoxAbic® elicits cross immunity against the coccidial species affecting broilers including immunity against E. acervuline, maxima, and tenella. It is used to immunize pullets before point of lay. The immune breeders transfer specific antibodies to the broilers through the egg yolk and shield them during early life after hatch while they are naturally exposed to the coccidia on the farm. This exposure brings about an immunity during maternal protection and transfers it to the broilers for the early stage of the broiler life cycle. Protective maternal antibodies are transferred via the egg yolk to offspring chicks, which hatch with high titers of maternal antibody. These maternal antibodies act to reduce oocyst shedding for the first 2-3 weeks of the chickens' growth period. This, in turn, leads to a 60-80% lowering of the peak litter oocyst counts, which usually occurs at 3-5 weeks of age.

Nevertheless, despite the fact that this vaccine is one that is able to transfer maternal immunity to broilers, and the broilers can be reared without coccidiostats in their feed, the immunity is an indirect immunity in that it is transferred from the mother to the broilers at an early stage in their life cycle. There is no mechanism for ensuring that the broilers retain the immunity and there are currently no available vaccines that can be administered directly to broilers or chicks after hatching. This leaves the possibility of spread of coccidiosis in broilers that have not adequately received immunity from the mother or older broilers that have lost the immunity and need a boost of immunity. Indeed, the manufacturers of CoxAbic® indicate that CoxAbic is used to vaccinate breeders and protect their broiler chicks only. The maternal immunity lasts for approximately 14 days or a little longer as determined by ELISA methods. If the birds are exposed to various species of Eimeria at later ages in the life cycle the maternal immunity no longer exists and the older birds remain be unprotected.

The CoxAbic® vaccine also suffers from the further drawback that it is administered by injection to the breeder chickens. In the vaccination schedule for CoxAbic® the pullets must be injected with the vaccine twice during their rearing with at least a 4 weeks interval between the two injections. The first injection can be done at the age of 12 to 15 weeks; the second injection at 18 to 21 weeks of age. Thus, the mode of administration of this vaccine is not readily adaptable for direct administration to large populations of broiler chickens.

As noted above, there is a major commercial incentive to obtain a vaccine for the treatment of broiler chickens. A vaccine that must be injected into chickens is impractical for administration to large populations of chickens. Therefore there is a need for a vaccine that may be readily administered to broiler chickens to effect protection against the deleterious effects of coccidiosis.

### BRIEF SUMMARY OF THE INVENTION

In certain aspects, the present invention addresses the need for a coccidiosis vaccine by providing a coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, a multiple cloning site for insertion of an open reading frame (ORF) to allow insertion of an ORF in frame with said hydrophobic signal sequence, a polyadenylation signal; and an avian adenovirus genome.

In specific embodiments, the ORF of interest encodes a truncated r56 antigen of Eimeria maxima. In other embodiments, the ORF of interest encodes a truncated TFP250 antigen of Eimeria maxima. In still additional embodiments, the ORF of interest encodes a truncated 82 kDa antigen of Eimeria maxima. In certain other exemplary embodiments, the multiple cloning site contains an ORF that encodes a truncated r56 antigen of Eimeria maxima, in combination with a truncated TFP250 antigen of Eimeria maxima and/or a truncated 82 kDa antigen of Eimeria maxima.

The coccidiosis vaccine may be prepared from any avian virus. Preferably, the coccidiosis vaccine employs an avian adenovirus genome selected from the group consisting of the genome of FAV 1, FAV 2, FAV 3, FAV 4, FAV 5, FAV 6, FAV 7, FAV 8, FAV 9, FAV 10, FAV 11 and FAV 12. In specific embodiments, the avian adenovirus genome is an FAV 8 genome.

The recombinant recombinant avian adenovirus vector further may comprise a cleavage sequence immediately upstream of the cloning site for the insertion of the ORF of interest, wherein expression product from said vector produces a soluble product.

In exemplary embodiments, the nucleic acid that encodes a truncated r56 comprises the sequence of nucleotides 70-1035 of the full length r56 sequence shown in SEQ ID NO:14 but does not encode the complete r56 protein sequence shown in SEQ ID NO:2. The nucleotide sequence encoded by residues 70-1035 is shown in SEQ ID NO:13. The full length Eimeria maxima R56 coding sequence also is shown in SEQ ID NO:14, which sequence is contained within the sequence of SEQ ID NO:1, where the atg start site is seen at residues 103-106. In still other embodiments, the nucleic acid that encodes a truncated r56 encodes the truncated r56 fragment that consists of amino acids 24-345 of SEQ ID NO:2 or a fragment of amino acids 24-345 of SEQ ID NO:2. In a specific alternative embodiments, the nucleic acid that encodes a truncated TFP250 comprises the sequence of nucleotides 6448-7083 of the full length TFP250 sequence shown in SEQ ID NO:16 but does not encode the complete TFP250 protein sequence shown in SEQ ID NO:4. More particularly, the nucleic acid that encodes a truncated TFP250 consists of the nucleic acid sequence of nucleotides 6448-7083 of SEQ ID NO:16. The nucleotide sequence encoded by residues 6448-7083 is shown in SEQ ID NO:15. The full length Eimeria maxima TFP250 coding sequence also is shown in SEQ ID NO:16, which sequence is contained within the sequence of SEQ ID NO:3, where the atg start site is seen at residues 231-233.

Also contemplated are compositions and methods of use of a coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated r56 that consists of amino acids 24-345 of SEQ ID NO:2 or a fragment of amino acids 24-345 of SEQ ID NO:2 inserted in frame with said hydrophobic signal sequence, a polyadenylation signal; and an avian adenovirus genome.

Another embodiment teaches preparation and use of a coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated TFP250 that consists of the nucleic acid sequence of nucleotides 6448-7083 of SEQ ID NO:16 inserted in frame with said hydrophobic signal sequence, a polyadenylation signal; and an avian adenovirus genome.

A further embodiment relates to compositions and methods of use of a coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated 82 kDa antigen of Eimeria maxima inserted in frame with said hydrophobic signal sequence, a polyadenylation signal; and an avian adenovirus genome

The invention also contemplates multivalent coccidiosis vaccine preparations that comprise combinations of the above coccidiosis vaccine compositions.

In addition the multivalent coccidiosis vaccine preparations may further comprise an immunogen selected from the group of consisting of Marek's Disease virus (MDV), Newcastle Disease virus (NDV), Infectious Bronchitis virus (IBV), Chicken Anaemia Virus (CAV), Infectious bursal disease virus (IBDV), Avian influenza (AI), Reo virus, Avian Retro virus, Fowl Adeno virus, Turkey Rhinotracheitis virus, Salmonella spp. and E. coli.

Exemplary methods of the invention relate to immunizing a subject against infection by Eimeria tenella, Eimeria maxima, Eimeria acervulina, Eimeria necatrix, Eimeria praecox, Eimeria mitis or Eimeria brunetti, comprising the step of administering to the subject a vaccine of the present invention. In specific embodiments, the administering elicits an enhanced level of immunity as compared to immunity seen when said subject is immunized with an FAV vector comprising a full length r56 or a full length TFP 250 antigen or a full length 82 kDa antigen.

The methods are used preferably for the treatment of an avian species selected from the group consisting of chickens, turkeys, geese, ducks, bantams, quail and pigeons. Preferably, the avian species is chickens. In specific embodiments, the chickens are adult broiler chickens.

The administration may be through any conventional route of administration including for example, spraying said subject with said vaccine, feeding said subject said vaccine in food, and providing said vaccine in the drinking supply of said subject.

Another method of the invention comprises a combination vaccination therapy for providing protective immunity against Eimeria tenella, Eimeria maxima, Eimeria acervulina, Eimeria necatrix, Eimeria praecox, Eimeria mitis or Eimeria brunetti, to a chicken population comprising the step of administering to the subject a vaccine of any of the present invention and administering CoxAbic® to said chicken population.

The combination therapy is such that the CoxAbic® is administered to breeder hens to confer immunity to chicks upon hatch and said vaccine of the invention is administered to the chicks at day 1 after hatching and later and to adult broiler hens of said population.

Other aspects of the invention describe an avian adenovirus vector comprising an avian adenovirus genome comprising a heterologous promoter, an heterologous hydrophobic signal sequence, a multiple cloning site, and a polyadenylation sequence, wherein said promoter and said hydrophobic signal sequence are located upstream of a multiple cloning site, wherein insertion of a ORF of interest into said multiple cloning site will result in an expression vector capable of expressing said ORF of interest under the control of said promoter and in frame with said signal sequence.

In specific embodiments, the hydrophobic signal sequence comprises a cleavage site to allow secretion of the expression product of said ORF of interest from host cell in which it is expressed. In other embodiments, the signal sequence does not contain a cleavage site thereby resulting in expression of a fused expression product of said ORF of interest being anchored to the cell surface of the host cell.

Also contemplated is a recombinant avian adenovirus vector comprising a promoter operably linked to a hydrophobic signal sequence comprising a nucleic acid that encodes a membrane anchoring domain, a multiple cloning site for insertion of a ORF of interest to allow insertion of a ORF of interest in frame with said hydrophobic signal sequence, a polyadenylation signal; and an avian adenovirus genome. The vector, in some embodiments, may further comprise a cleavage sequence immediately upstream of the cloning site for the insertion of the ORF of interest, wherein expression product from said vector produces a soluble ORF product.

Also disclosed is a recombinant avian adenovirus vector comprising a promoter operably linked to a hydrophobic secretion signal sequence and cleavage site, a nucleic acid that encodes a truncated r56 protein of Eimeria maxima, a polyadenylation signal and an avian adenovirus genome.

Another embodiment relates to a recombinant avian adenovirus vector comprising a promoter operably linked to a hydrophobic secretion signal sequence and cleavage site, a nucleic acid that encodes a truncated TFP250 protein of Eimeria maxima, a polyadenylation signal and an avian adenovirus genome.

Yet a further embodiment relates to a recombinant avian adenovirus vector comprising a promoter operably linked to a hydrophobic secretion signal sequence and cleavage site, a nucleic acid that encodes a truncated 82 kDa protein of Eimeria maxima, a polyadenylation signal and an avian adenovirus genome.

Still a further embodiment relates to a recombinant avian adenovirus vector comprising a promoter operably linked to a signal sequence comprising a nucleic acid that encodes a membrane anchoring domain, a nucleic acid that encodes a truncated r56 protein of Eimeria maxima, a polyadenylation signal and an avian adenovirus genome.

Yet additional embodiments describe a recombinant avian adenovirus vector comprising a promoter operably linked to a signal sequence comprising a nucleic acid that encodes a membrane anchoring domain, a nucleic acid that encodes a truncated TFP250 protein of Eimeria maxima, a polyadenylation signal and an avian adenovirus genome.

Also contemplated is a recombinant avian adenovirus vector comprising a promoter operably linked to a signal sequence comprising a nucleic acid that encodes a membrane anchoring domain, a nucleic acid that encodes a truncated 82 kDa protein of Eimeria maxima, a polyadenylation signal and an avian adenovirus genome.

In the above outlined vaccines, the nucleic acid that encodes a truncated r56 comprises the sequence of nucleotides 70-1035 of the full length r56 sequence shown in SEQ ID NO:14 but does not encode the complete r56 protein sequence shown in SEQ ID NO:2. More particularly, the nucleic acid that encodes a truncated r56 consists of the nucleic acid sequence of nucleotides 70-1035 of SEQ ID NO:14, or a fragment of nucleotides 70-1035 of SEQ ID NO:14. For example, the nucleic acid that encodes a truncated r56 encodes the truncated r56 fragment that consists of amino acids 24-345 of SEQ ID NO:2 or a fragment of amino acids 24-345 of SEQ ID NO:2.

In other embodiments, the nucleic acid that encodes a truncated TFP250 comprises the sequence of nucleotides 6448-7083 of the full length TFP250 sequence shown in SEQ ID NO:3 but does not encode the complete TFP250 protein sequence shown in SEQ ID NO:4. More particularly, the nucleic acid that encodes a truncated TFP250 consists of the nucleic acid sequence of nucleotides 6448-7083 of SEQ ID NO:16.

The recombinant avian adenovirus vector that produce secreted can comprise any secretion signal sequence. In specific embodiments, the secretion signal sequence is selected from the group consisting of the secretion signal sequence of chicken gamma interferon, porcine gamma interferon, and Human Influenza H1N2.

The recombinant avian adenovirus vector that produce anchored products can comprise any membrane anchoring signal sequence. In specific embodiments, the membrane anchoring signal sequence is selected from the group consisting of the secretion signal sequence of an avian influenza HA antigen.

Any of the expression vectors described may readily be formulated into vaccines for use in the methods described herein.

Exemplary methods comprise methods of eliciting an immune response in an avian population comprising administering to said population such a vaccine.

Preferred methods of vaccinating a fowl population against coccidiosis comprise administering a vaccine comprising a recombinant avian adenovirus vector of the present invention, wherein administration of said vaccine elicits an increased immune response as compared to administration of a vaccine comprising full length r56 or full length TFP250.

Also contemplated are isolated cells that comprise recombinant avian adenovirus vectors described herein.

Any of the recombinant avian adenovirus vectors may be advantageously combined with a suitable excipient to produce a pharmaceutical formulation for the treatment of animals and in particular, birds.

### BRIEF DESCRIPTION OF SEVERAL VIEWS OF THE DRAWINGS

FIG. 1 shows a schematic of the FAV based vectors of the invention.

FIG. 2 shows Western blot analysis of various FAV constructs comprising r56 protein in either native, membrane anchored or secreted form.

FIG. 3 shows Western blot analysis of various FAV constructs comprising TFP250 protein in either native, membrane anchored or secreted form.

FIG. 4. shows a collection of eukaryotic signal sequences reproduced from FIG. 1 of Heijne Eur. J. Biochem 133, 17-21 (1983). The sequences are aligned based on their known or predicted cleavage sites, which are indicated by an asterisk (*). The sequences shown herein are SEQ ID NO:35-124.

FIG. 5 shows scheme for chemical synthesis of expression cassettes.

FIG. 6 shows scheme for PCR amplification for preparing constructs.

FIG. 7 shows scheme for use of multiple cloning site for insertion of sequences.

FIG. 8 shows the plasmid structure of CMVP-TFP250-pA/1054 (FAV RHE) with secretion signal of gamma interferon, previously shown in the Appendix as “p1232_entire”. The entire sequence is depicted as SEQ ID NO:17 herein. In that sequence, the secretion signal sequence is encoded by SEQ ID NO:18 which is located at nucleotides 5629 . . . 5712 of SEQ ID NO:17 and translates into the protein of SEQ ID NO:19. The truncated TFP250 insert is encoded by the sequence of SEQ ID NO:20 which is located at nucleotides 5713 to 6348 of SEQ ID NO:17 and translates to the sequence of SEQ ID NO:21. The plasmid has a CMV promoter sequence at location 4965 . . . 5623. The information depicted in this Figure and the associated sequence information was previously presented in the Appendix of priority application, U.S. Provisional Patent Application No. 61/122,596, which was filed on Dec. 15, 2008 (incorporated herein by reference in its entirety.

FIG. 9 shows the plasmid structure of MLP-R56-pA-pA/1054 (FAV RHE) with secretion signal of gamma interferon, previously shown in the Appendix as “p1223_entire”. The entire sequence is depicted as SEQ ID NO:22 herein. In that sequence, the secretion signal sequence is encoded by SEQ ID NO:23 which is located at nucleotides 5381.5461 of SEQ ID NO:22 and translates into the protein of SEQ ID NO:6. The truncated R56 insert is encoded by the sequence of SEQ ID NO:24 which is located at nucleotides 5462 to 6430 of SEQ ID NO:22 and translates to the sequence of SEQ ID NO:25. The plasmid has a MLP sequence at nucleotides 5381 . . . 5461. The information depicted in this Figure and the associated sequence information was previously presented in the Appendix of priority application, U.S. Provisional Patent Application No. 61/122,596, which was filed on Dec. 15, 2008 (incorporated herein by reference in its entirety.

FIG. 10A through FIG. 100 shows the sequence of the 82 kDa protein of E. maxima. The sequences shown in this figure are SEQ ID NO:26 (upper strand); SEQ ID NO:27 (lower strand) and SEQ ID NO:28 (protein sequence).

FIG. 11 shows a comparison of the R56 sequence of E. maxima (SEQ ID NO:2), the R56 sequence of E. tenella (SEQ ID NO:29). The sequence of a truncated R56 lacking the signal sequence (SEQ ID NO:30) also is depicted. The lower portion of the figure shows an alignment of a tuncated R56 from E. tenella (SEQ ID NO:31) with a truncated R56 from E. maxima (SEQ ID NO:32). This sequence information figure was previously presented in the Appendix of priority application, U.S. Provisional Patent Application No. 61/122,596, which was filed on Dec. 15, 2008 (incorporated herein by reference in its entirety.

FIG. 12 shows the shortened versions of R56 of E. maxima (SEQ ID NO:33) and E. tenella (SEQ ID NO:34). This sequence information figure was previously presented in the Appendix of priority application, U.S. Provisional Patent Application No. 61/122,596, which was filed on Dec. 15, 2008 (incorporated herein by reference in its entirety.

### DETAILED DESCRIPTION OF THE INVENTION

The present invention relates to methods of preparing and use of recombinant viral vaccine compositions that can be administered to a population of broiler chickens for protective immunity of such birds against coccidiosis. This vaccine can be administered to the birds after hatching and does not require administration by injection but instead may be administered orally, through the food supply, the drinking supply or even as an aerosol spray. An advantage of the vaccine constructs of the invention is that they direct expression of the immunogen being delivered to an extracellular site on the infected cell rather than internal expression of the immunogen. In the case of the vaccines described herein, the immunogen is thus delivered to the outer surface of mucosal cells (e.g., mucosal cells in the nasal passages, the respiratory tract, the gastrointestinal tract, the intestinal mucosa and the like) thereby presenting the immunogen at a site where an immune response may rapidly be mounted as opposed to expression of the delivered immunogen within the cells where it may not come into efficient contact with the appropriate immune response machinery.

The existing vaccines do not meet the long-felt need in the art for an effective coccidiosis vaccine for a number of reasons. Firstly, CoxAbic® the currently available vaccine for treating coccidiosis only confers immunity to the mother and relies on transfer of that immunity to broiler population through the egg yolk. The maternal immunity lasts for a relatively short period of about the first 14 days after egg hatch. Thus it is not an effective vaccine for eliciting a long term response specifically in broiler chickens. Moreover, the vaccine is administered via injection. Again, this renders the vaccine ineffective for treating large populations of older birds.

To combat the problems with the existing treatments for coccidiosis, the present inventors have developed a new vaccine for conferring protective immunity to broilers. The vaccine is based on an avian adenovirus expression system that affords expression of an Eimeria antigen in a subunit vaccine. The antigen is expressed in-frame with a hydrophobic signal sequence and is either presented on the cell surface of virus-infected cells in the chicken to which the vaccine has been administered or is alternatively secreted into the extracellular domain in such infected chickens in the event that the expression vector is one in which the hydrophobic signal sequence also comprises a cleavage signal. These features and methods and compositions for using recombinant avian adenovirus coccidiosis vaccines are described in further detail herein below.

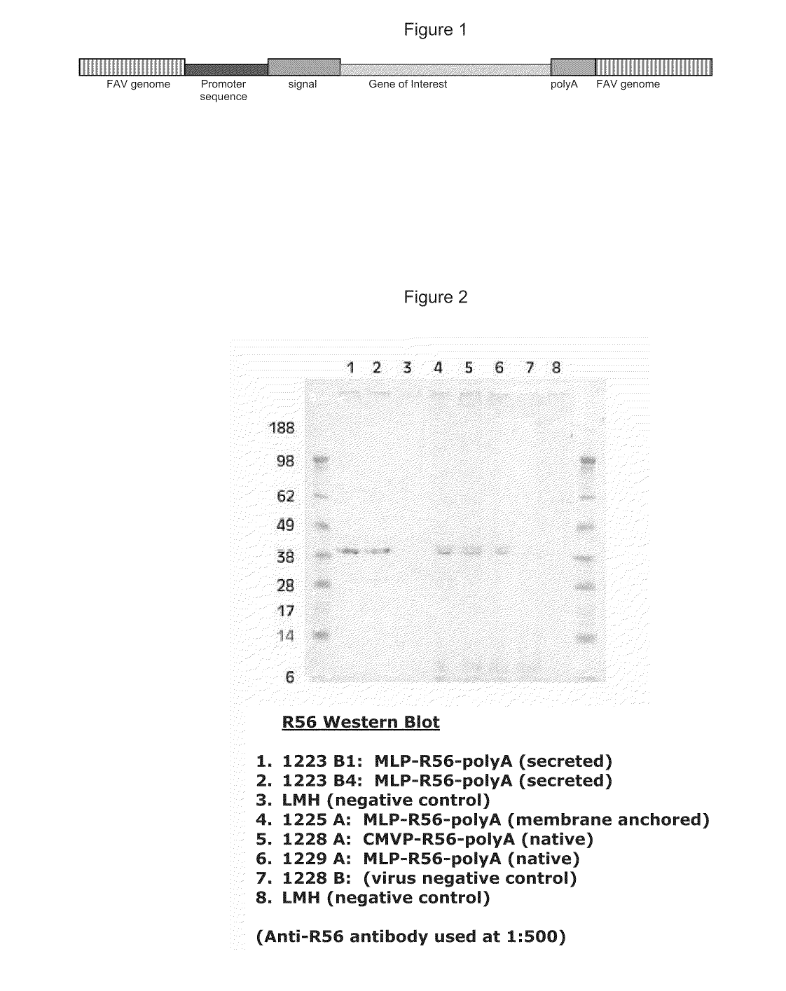

In general terms the vaccine of the present invention is comprised of an expression vector that is made of an avian adenovirus genome and is organized as shown in FIG. 1. Avian or fowl adenovirus (FAV) are well known to those of skill in the art and have been extensively characterized. For example, an avian adenovirus, termed fowl adenovirus type 1 strain CELO (for chick embryo lethal orphan), has been described (Chiocca, S. et al., J. Virol. 70:2939-49 (1996); Li, P. et al., J. Gen. Virol. 65 (Pt 10):1817-25 (1984); May, J. T. et al., Virology 68:483-9 (1975); Lehrmann, H., Cotton, M., J. Virol. 73:6517-25 (1999); Chiocca, S. et al., J. Virol. 71:3168-77 (1997)). The use and methods of manipulating CELO to form vectors for gene therapy and for use in vaccines against infectious diseases in humans and animals and particularly birds has also been extensively described in e.g., U.S. Pat. No. 6,335,016. The complete genome sequence of CELO (FAV 1 or FAV A) may be found at Genbank Accession Nos. U46933; NC_{—}001720, and AC_{—}000014.

In specific embodiments, the fowl adenovirus vector used in the methods and compositions described herein is a fowl adenovirus vector (FAV), such as described in U.S. Ser. Nos. 08/448,617 and 09/272,032, the contents of which are incorporated herein by way of reference. In a particularly preferred embodiment, the vector comprises the right-hand end of FAV serotype 8 (hereinafter “FAV8”). The entire nucleotide sequence of the FAV8 is set forth herein as SEQ ID NO: 5. The entire nucleotide sequence of the FAV8 expression vector is also contained in GenBank Accession No. AF155911. Method for the isolation and production of FAV 8 are described in U.S. Pat. No. 6,296,852 (incorporated herein by reference in its entirety).

FAV 9 (also referred to as FAV D) is described in Cao et al., J. Gen. Virol. 79 (Pt 10), 2507-2516 (1998) and the complete genome thereof is shown at GenBank Accession Nos. AF083975 and NC_{—}000899.

Given the teachings of the sequences of FAVs known to those of skill in the art, the vaccines of the present invention may readily be prepared using FAV 1, FAV 2, FAV 3, FAV 4, FAV 5, FAV 6, FAV 7, FAV 8, FAV 9, FAV 10, FAV 11, FAV 12 or any subsequently isolated serotype of fowl adenovirus (see Monreal, G. Adenoviruses and adeno-associated viruses of Poultry. Poultry Science Rev. 4, p. 1-27 (1992) for virus classification). AS described in U.S. Pat. No. 6,296,852, FAV CFA20 (which is an FAV serotype 10), CFA15 (serotype 10) and CFA 40 and CFA 44 (both serotype 8) and FAV CFA15 and CFA19 (serotype 9) may be particularly useful for vaccine production.

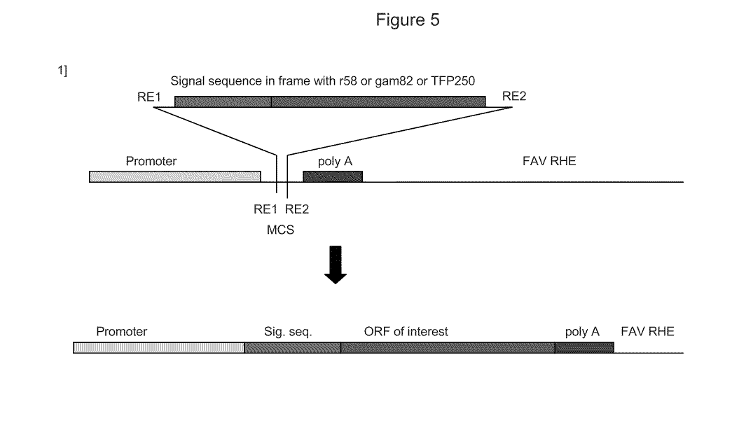

In the vaccines prepared herein the promoter used may be any promoter that can drive expression of a heterologous coding region of interest in an FAV construct. Such promoters include but are not limited to avian adenoviral major late promoter (MLP), CMVp, PGK-, E1-, SV40 early promoter (SVG2), SV40 late promoter, SV-40 immediate early promoter, T4 late promoter, and HSV-1 TK (herpesvirus type 1 thymidine kinase) gene promoter, the RSV (Rous Sarcoma Virus) LTR (long terminal repeat) and the PGK (phosphoglycerate kinase) gene promoter. The DNA sequence of the FAV MLP is shown in FIG. 5 of U.S. Pat. No. 6,296,852. Many other mammalian or avian promoters are known to those of skill in the art and also may be used.

The promoter used in the vaccines described herein drives the expression of an in-frame fusion of a hydrophobic signal sequence linked in-frame with a nucleic acid sequence of an open reading frame or coding region of interest. The hydrophobic signal sequence may be any sequence that can be used to target or specifically direct the expression of the pen reading frame or coding region of interest to the outer membrane of the host cell that is infected with the fowl adenoviral expression vector. In the present invention the FAV-based expression vector is intended to infect chickens. The FAV typically infects mucosal, liver and epithelial cells of the chicken which may be found for example in the intestinal tract, the respiratory tract or the gastrointestinal tract of the chicken. Thus, the hydrophobic signal sequence is one which traffics the expression of the open reading frame or coding region of interest on the cell surfaces of these mucosal cells. By thus presenting the open reading frame or coding region of interest at the cell surface of mucosal cells in the animal, the vaccine of the invention are able to most effectively deliver the antigen to the internal site where an immune response can be effectively mounted as opposed to expression within the cell of animal where it may be less effective at facilitating the mounting of an immune response. This extracellular secretion of the expression products through the use of the currently described vaccines leads to a greater antibody immune response and antibody production than is seen when the vaccine is prepared with wild-type immunogens.

In eukaryotic cells, secretory proteins are targeted to the endoplasmic reticulum membrane by hydrophobic signal sequences. The present invention uses this property to employ heterologous hydrophobic signal sequences to direct the expression of a given protein in the vaccine to the cell surface.

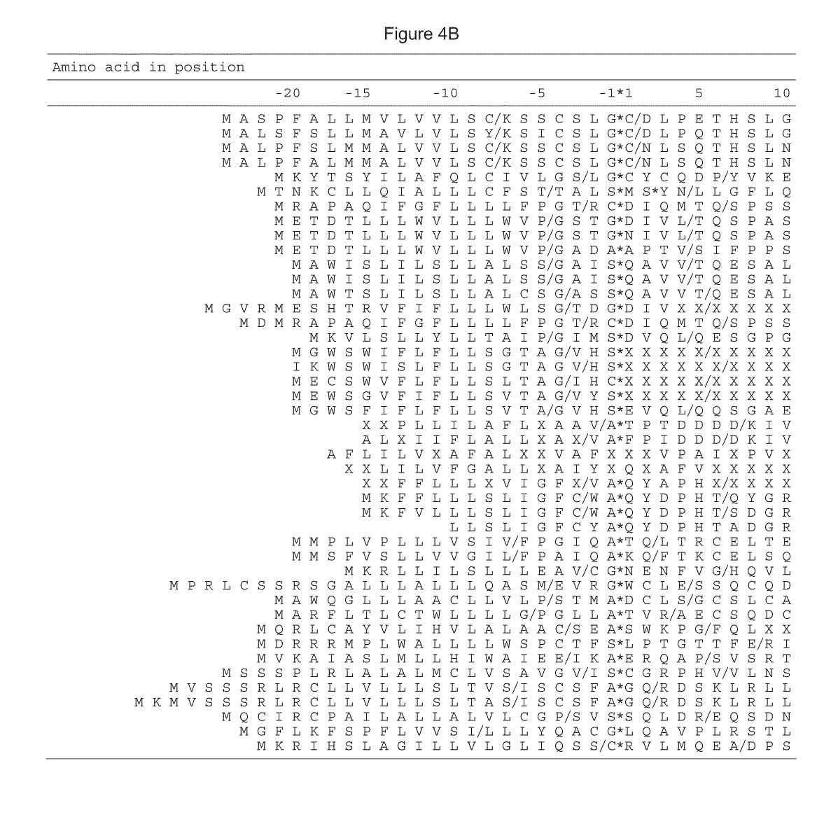

The viral vectors employed herein are recombinant vectors in that they comprise a polynucleotide construct that contains nucleic acid that encodes a modified ORF in which the expression product of the ORF allows secretion (from the infected cell) of truncated ORF protein upon expression or directly expresses the protein on the surface of the infected cell. For example, the ORF of interest is expressed in-frame with the signal sequence from chicken gamma interferon, porcine gamma interferon, or the HA protein of influenza virus. Other signal sequences that may be used include, for example, the signal sequence of whey phosphoprotein signal sequence; α-1 acid glycoprotein; α-thyrotropin; insulin from hagfish; insulin from anglerfish; human insulin; rat insulin I or II; ovine β-casein; ovine X-casein; ovine α-lactalbumin; ovine β-lactoglobulin; ovine α-s1 casein, and ovine α-s2 casein; VS virus glycoprotein; cockerel VLDL-11; bee melittin; rat lactin; human placental lactogen; human β-choriogonadotropin; human α-choriogonadotropin; rabbit uteroglobin; rat growth hormone; human growth hormone, human; bovine growth hormone; bovine parathyroid hormone; rat relaxin; rat serum albumin; human serum albumin; rat liver albumin; chicken tropoelastin B; chicken ovomucoid; chicken lysozyme; chicken conalbumin; human α-1 antitrypsin; rat prostatic binding protein; rat prostatic binding protein c2; AD virus glycoprotein; rat apolipoprotein Al; rabies virus glycoprotein; human influenza Victoria hemagglutinin; human influenza Jap hemagglutinin; avian influenza FPV hemagglutinin; human leukocyte interferon; human immune interferon; human fibroblast interferon; mouse X-immunoglobulin; mouse λ-immunoglobulin; mouse X-immunoglobulin; mouse H-chain immunoglobulin; mouse embryonic VH-immunoglobulin; mouse H-chain immunoglobulin; canine trypsinogen 1; canine trypsinogen 2+3; canine chymotrypsinogen 2; canine carboxypeptidase Al; canine amylase; mouse amylase; rat amylase; rabbit α-lactalbumin; porcine α-lactalbumin; rat carboxypeptidase A; bovine ACTH-β-LPH precursor; porcine ACTH-β-LPH precursor; human ACTH-β-LPH precursor; porcine gastrin; mouse renin; trypanosome glycoprotein; catfish somatostatin; anglerfish somatostatin; rat calcitonin; and anglerfish glucagons. Each of these signal sequences is shown at FIG. 1 of von Heijne et al. Eur. J. Biochem 133 17-21 (1983) and may readily be adapted for use herein. The signal sequences from FIG. 1 of the aforementioned reference are reproduced in FIG. 4 herein.

These and other signal peptide sites for a given protein can readily be determined using methods known to those of skill in the art. For example, signal peptide site can be predicted using the SignalP 3.0 server (Bendtsen, J. D., Nielsen, H., von Heijne, G. & Brunak, S. (2004) Improved prediction of signal peptides: SignalP 3.0. J. Mol. Biol. 340, 783-795). Additionally, there are websites available to facilitate determination of signal sequences see e.g., http://www.cbs.dtu.dk/services/SignalP/. The exact identity of the signal sequence used is not important as long as it is a hydrophobic sequence that is capable of trafficking the expressed product to the cell surface.

In preferred embodiments, the signal sequence contains a cleavage site that permits the signal sequence to be cleaved and allows the attached protein to be secreted extracellular space of such cells. In particularly preferred embodiments, this aspect of the invention is demonstrated using the signal sequences from chicken gamma IFN which contains sequence: MTCQTYNLFVLSVIMIYYGHTASSLNL (SEQ ID NO:6) encoded by the DNA sequence of ATG ACT TGC CAG ACT TAC AAC TTG TTT GTT CTG TCT GTC ATC ATG ATT TAT TAT GGA CAT ACT GCA AGT AGT CTA AAT CTT (SEQ ID NO:7), a hydrophobic signal sequence for porcine gamma IFN is: MSYTTYFLAFQLCVTLCFSGSYC (SEQ ID NO:8), which is encoded by the DNA sequence of ATG AGT TAT ACA ACT TAT TTC TTA GCT TTT CAG CTT TGC GTG ACT TTG TGT TTT TCT GGC TCT TAC TGC (SEQ ID NO:9), a hydrophobic signal sequence for human influenza virus H1N2 is: MKVKLLILLCTFTATYADTI (SEQ ID NO:10) encoded by a sequence of: atg aaa gta aaa cta ctg atc ctg tta tgt aca ttt aca get aca tat gca gac aca ata (SEQ ID NO:11). Each of these exemplary sequences also contain a cleavage site at which a signal peptidase acts and results in the release of the expressed ORF.

In addition to the promoter and the hydrophobic signal sequence, which may or may not contain a cleavage site, the expression vector further comprises a polyadenylation sequence. The polyA tail protects the mRNA molecule from degradation by exonucleases in the cytoplasm and aids in transcription termination, export of the mRNA from the nucleus, and translation. Almost all eukaryotic mRNAs are polyadenylated. Those skilled in the art routinely add a polyA tail sequence for the recombinant expression of proteins.

The ORF or other exogenous sequences inserted into the vectors of the present invention may be any ORF or other exogenous sequences that is desired to be expressed through the use of a FAV vector described herein. These other exogenous sequences can consist of one or more ORFS or expression products of interest or other nucleotide sequences that are not genes but have other functions of therapeutic interest.

However, in specific embodiments, the present invention describes vectors that are to be used as subunit vaccines for vaccinating chickens against coccidiosis. In this context the ORF interest is one that encodes an antigen of the apicomplexan protozoan parasite Eimeria. More specifically, the ORF is selected from the group consisting of antigens of 56 kDa, 82 kDa and 230 kDa as described in U.S. Pat. No. 7,423,137 (incorporated herein by reference). More particularly, it has been found by the present inventors than incorporation of the full length sequence of the 56 kDa (alternatively referred to herein as r56) or the 230 kDa (alternatively referred to herein as TPF250) antigen into FAV does not produce an effective vaccine. However, when a truncated r56 sequence is used the vaccine is effective at eliciting an immune response. The full length amino acid sequence of r56 is shown in SEQ ID NO:2, this protein sequence is encoded by a sequence of SEQ ID NO:1, the r56 encoding full length gen also is also depicted in SEQ ID NO:14. In addition, a plasmid encoding the 56 kDa antigen is publicly available from the Australian Government Analytical Laboratories, Pymble, Australia, under Accession No. NM01/22400. The bacterial cell transformed with the 56 kDa antigen also is available from the same depository under Accession No. NM01/22401. In specific embodiments therefore, a truncated r56 is used for the preparation of a coccidiosis vaccine. The sequence of r56 is one which comprises amino acids 24-345 of SEQ ID NO:2 which may be encoded by a sequence of 70-1035 of SEQ ID NO:14. In a preferred coccidiosis vaccine of the invention, the truncated sequence of r56 is in frame with a hydrophobic signal sequence that anchors the truncated r56 to the cell surface of the infected cell. In another preferred coccidiosis vaccine of the invention, the truncated sequence of r56 is in frame with a hydrophobic signal sequence that comprises a cleavage site such that the upon trafficking to the extracellular side of the membrane, the truncated r56 is released into the extracellular space of the cell. In specific embodiments, a coccidiosis vaccine is provided in which the anchored r56 protein is anchored to the cells surface through a hydrophobic signal sequence of an avian influenza HA antigen. In still other specific embodiments, a coccidiosis vaccine is provided in which the hydrophobic signal sequence containing a cleavage site is selected from the group consisting of chicken gamma interferon, porcine gamma interferon, and human influenza virus H1N2. In certain embodiments, a coccidiosis vaccine composition may be prepared that comprises both types of vaccines, i.e., a vaccine that allows cell surface expression of the truncated r56 and a vaccine that releases the expressed r56 into the extracellular space.

In yet other exemplary embodiments, a truncated TFP250 is used for the preparation of a coccidiosis vaccine. The full length sequence of TFP250 is shown in SEQ ID NO:4 and is encoded by SEQ ID NO:3 or SEQ ID NO:16. A plasmid encoding the 250 kDa antigen is publicly available from the Australian Government Analytical Laboratories, Pymble, Australia under Accession No. NM01/22396. A bacterial cell transformed with this antigen is available from the same depository under Accession No. NM01/22397. The sequence of TFP250 used in the preferred vaccines herein is one which comprises amino acids 2149-2361 or amino acids 2150-2361 of SEQ ID NO:4 which may be encoded by a sequence of 6444-7083 and 2149-2361, respectively of SEQ ID NO:16. In a preferred coccidiosis vaccine of the invention, the truncated sequence of TFP250 is in frame with a hydrophobic signal sequence that anchors the truncated TFP250 to the cell surface of the infected cell. In another preferred coccidiosis vaccine of the invention, the truncated sequence of TFP250 is in frame with a hydrophobic signal sequence that comprises a cleavage site such that the upon trafficking to the extracellular side of the membrane, the truncated TFP250 is released into the extracellular space of the cell. In specific embodiments, a coccidiosis vaccine is provided in which the anchored r56 protein is anchored to the cells surface through a hydrophobic signal sequence of an avian influenza HA antigen. In still other specific embodiments, a coccidiosis vaccine is provided in which the hydrophobic signal sequence containing a cleavage site is selected from the group consisting of chicken gamma interferon, porcine gamma interferon, and human influenza virus H1N2. In certain embodiments, a coccidiosis vaccine composition may be prepared that comprises both types of vaccines, i.e., a vaccine that allows cell surface expression of the truncated TFP250 and a vaccine that releases the expressed TFP250 into the extracellular space.

In like manner, it also is contemplated that the vaccines also may be prepared in conjunction with vaccines that express the 82 kDa antigen of Eimeria. The 82 kDa (also referred to herein as gam82) antigen is publicly available from the Australian Government Analytical Laboratories, Pymble, Australia under Accession No. NM01/22398 and a bacterial cell transformed with the 82 kDa antigen is available from the same depository at Accession No. NM01/22399 (and is shown in the appendix herein). Any of the vaccines of the present invention may be used in combination with existing vaccination protocols. For example, the vaccines described herein may be used in combination with CoxAbic® to produce protective immunity in breeders, chicks and in older chickens.

While many of the examples described herein relate to vaccines prepared from antigens of Eimeria maxima, it will be readily apparent that the skilled person may prepare such vaccines using homologous sequences from other Eimeria species. The skilled person can readily identify such appropriate DNA sequences via homology to the above sequences of the open reading frames from Eimeria maxima using conventional molecular biology techniques. Thus homologs of Eimeria maxima r56, TFP25 and 82 kDa ORFS from other Eimeria species, e.g., Eimeria tenella, Eimeria acervulina, Eimeria necatrix, Eimeria praecox, Eimeria mitis or Eimeria brunetti are specifically contemplated for the preparation of coccidiosis vaccines described herein. In this regard, SEQ ID NO:12 provides the sequence of r56 of Eimeria tenella. Given that the r56 sequence of Eimeria tenella and Eimeria maxima are shown to be effective, the skilled person will readily be able to identify homologs of r56 from other Eimeria species for use in the methods and compositions described herein.

In preferred embodiments, the vaccines of the invention are used to provide a method of immunizing a subject against infection by Eimeria tenella, Eimeria maxima, Eimeria acervulina, Eimeria necatrix, Eimeria praecox, Eimeria mitis or Eimeria brunetti, or a microorganism expressing an immunologically cross-reactive antigen, comprising the step of administering to the subject the vaccine of the subject invention. In particularly preferred embodiments, the subject is an avian species including but not limited to an avian species selected from the group consisting of chickens, turkeys, geese, ducks, bantams, quail and pigeons. In particularly preferred embodiments, the avian species is chickens, and more specifically broiler chickens.

The vaccine may be administered through any route typically employed for vaccination including, but not limited to, systemically (for example, intravenously, intratracheally, intravascularly, intrapulmonarilly, intraperitoneally, intranasally, parenterally, enterically, intramuscularly, subcutaneously, intratumorally or intracranially), by oral administration, by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the ORF or fragment of polypeptide of interest), but can be determined by one of skill in the art.

The vaccine may be administered according to a conventional administration regimen, e.g., as a single or repeated administration in a manner compatible with the dosage formulation, and in such amount as will be prophylactically effective, i.e. the amount of immunizing antigen or recombinant micro-organism capable of expressing said antigen that will induce immunity in birds (especially poultry) against challenge by virulent Eimeria parasites. Immunity is defined as the induction of a significant level of protection in a population of birds after vaccination compared to an unvaccinated group. In specific embodiments, the immunity conferred is an enhanced immunity wherein the vaccines of the invention induce a level of protection in a population of birds after vaccination that is more effective than the protection seen when the birds are vaccinated with a subunit FAV vector comprising a full length r56 or a full length TFP 250 antigen or a full length 82 kDa antigen. This more effective vaccination is observed due to the current subunit vaccines have a greater stability than subunit vaccines prepared from the full length sequences.

A vaccine of the invention may reduce the number of oocysts shed by the infected animals. Normally, the shed oocysts will infect other animals in the flock. A decrease in the number of oocysts shedded will then also give a decrease in the number of animals which is subsequently infected and also a decrease in the number of oocysts shed will give rise to a lesser infectious load. In specific embodiments, the vaccines of the present invention reduce the number of cecal lesions in a bird when challenged with a subsequent Eimeria infection.

Typically, live viral vector vaccines the dose rate per chicken may range from 10² to 10¹⁰ pfu (but even <1000 pfu might be sufficient e.g. for HVT).

The vaccines of the invention may also be effectively mixed with other antigenic components of the same and/or other Eimeria species, and/or with additional immunogens derived from a poultry pathogenic virus or micro-organism and/or nucleic acid sequences encoding these immunogens. Such a combination vaccine can decrease the parasitic load in a flock of birds and can increase the level of protection against coccidiosis, and in addition protect against other poultry pathogens. Such other immunogens may include e.g. be selected from the group of poultry pathogenic viruses or micro-organisms consisting of Marek's Disease virus (MDV), Newcastle Disease virus (NDV), Infectious Bronchitis virus (IBV), Chicken Anaemia Agent (CM), Reo virus, Avian Retro virus, Fowl Adeno virus, Turkey Rhinotracheitis virus, Salmonella spp. or E. Coli. Thus, multivalent vaccines are contemplated by the present invention. Particularly preferred multivalent vaccines are those coccidiosis vaccines of the present invention that are comprised of the above-described r56 expressing vectors in combination with the above-described TFP250 expressing fowl adenovirus vectors, and/or in combination with the above-described 82 kDa antigen expressing fowl adenovirus vectors.

A particular advantage of the vaccines of the present invention which are prepared from truncated antigens of Eimeria maxima expressed in the FAV-based subunit vaccines is that they can be administered through an aerosol spray or through eye drops or even be administered in the drinking water, in ovo, or in the bird feed of the broiler birds or formulated as a gel matrix to be ingested by the birds thereby making these vaccines applicable to large scale vaccination of broiler birds even under typical over-crowded conditions. Thus preferably, the vaccine may be prepared with excipients that facilitate spraying of the vaccine to achieve administration thereof.

These vaccines of the invention may be sprayed onto or fed to newly hatched chicks and they may be likewise sprayed and fed to older birds.

The vaccines of the invention are capable of protecting poultry against the pathogenic effects of Eimeria infection in a manner that creates a more pronounced immune response and confers better immunity than a like vaccine created with a full length sequence of r56 or a full length sequence of TFP250.

Vaccines according to the present invention can be made e.g. by merely admixing the fowl adenovirus vectors described above with a pharmaceutically acceptable carrier. A pharmaceutically acceptable carrier is understood to be a compound that does not adversely effect the health of the animal to be vaccinated, at least not to the extend that the adverse effect is worse than the effects seen due to illness when the animal is not vaccinated. A pharmaceutically acceptable carrier can be e.g. sterile water or a sterile physiological salt solution. In a more complex form, the carrier can e.g. be a buffer.

Alternatively, the coccidiosis vaccines of the present invention also may contain an adjuvant. Adjuvants in general comprise substances that boost the immune response of the host in a non-specific manner. A number of different adjuvants are known in the art. Examples of adjuvants are Freunds Complete and Incomplete adjuvant, vitamin E, non-ionic block polymers and polyamines such as dextransulphate, carbopol and pyran. Also very suitable are surface active substances such as Span, Tween, hexadecylamine, lysolecitin, methoxyhexadecylglycerol and saponins such as Quill A®. Furthermore, peptides such as muramyldipeptides, dimethylglycine, tuftsin, are often used. Next to these adjuvants, Immune-stimulating Complexes (ISCOMS), mineral oil e.g. Bayol® or Markol®, vegetable oils or emulsions thereof and Diluvac® Forte can advantageously be used. The vaccine may also comprise a “vehicle”. A vehicle is a compound to which the polypeptide adheres, without being covalently bound to it. Often used vehicle compounds are e.g. aluminium hydroxide, -phosphate, sulphate or -oxide, silica, Kaolin, and Bentonite. A special form of such a vehicle, in which the antigen is partially embedded in the vehicle, is the so-called ISCOM (EP 109.942, EP 180.564, EP 242.380).

The vaccine composition may further comprise stabilisers, e.g. to protect degradation-prone polypeptides from being degraded, to enhance the shelf-life of the vaccine, or to improve freeze-drying efficiency. Useful stabilisers include skimmed milk, gelatin, bovine serum albumin, carbohydrates e.g. sorbitol, mannitol, trehalose, starch, sucrose, dextran or glucose, proteins such as albumin or casein or degradation products thereof, and buffers, such as alkali metal phosphates.

Freeze-dried material can be stored and kept viable for many years. Storage temperatures for freeze-dried material may well be above zero degrees, without being detrimental to the material. In certain aspects, the vaccines are freeze dried.

### EXAMPLES

The following examples demonstrate the production of vaccines according to the present invention. In these examples, fowl adenovirus serotype 8 (FAV8) was used. A major benefit of the use of this delivery system lies in its ability to utilize attenuated FAV8 as a vector for subunit vaccine delivery to the appropriate target tissue (in this case the intestinal mucosa).

### Example 1

### Preparation and Analysis of Constructs

In the present example, one of the most immunogenic proteins from the macrogametocyte stage of Eimeria parasites, the recombinant protein—r56, was cloned into an FAV8 vector. In addition, another immunogenic protein from the Eimeria merogony stage, the recombinant protein—TFP250, was separately cloned into another FAV8 vector. This TFP250 ORF encodes a portion of a microneme protein (an organelle involved in parasite invasion), that in previous studies was shown to also induce partial protective immunity against a challenge infection with Eimeria.

The expression constructs formed are shown in the following two Tables

Portions of R56 gene as used in the Coccidiosis constructs:

| | R56 encoding nucleic | |
|---|---|---|
| Construct | acid insert | R56 amino acid encoded |
| 1222 CMVp-R56-pA | nucleotides 70-1035 of | amino acids 24-345 of |
| Secreted | SEQ ID NO: 14 | SEQ ID NO: 2 |
| 1224 CMVp-R56-pA | nucleotides 73-1035 of | amino acids 25-345 of |
| Membrane | SEQ ID NO: 14 | SEQ ID NO: 2 |
| 1228 CMVp-R56-pA | nucleotides 70-1035 of | amino acids 24-345 of |
| Native | SEQ ID NO: 14 | SEQ ID NO: 2 |
| 1223 MLP-R56-pA | nucleotides 70-1035 of | amino acids 24-345 of |
| Secreted | SEQ ID NO: 14 | SEQ ID NO: 2 |
| 1225 MLP-R56-pA | nucleotides 73-1035 of | amino acids 25-345 of |
| Membrane | SEQ ID NO: 14 | SEQ ID NO: 2 |
| 1229 MLP-R56-pA | nucleotides 70-1035 of | amino acids 24-345 (of |
| Native | SEQ ID NO: 14 | SEQ ID NO: 2 |

Portions of TFP250 gene as used in the Coccidiosis constructs

| | TFP250 encoding nucleic | TFP250 amino acid |
|---|---|---|
| Construct | acid insert | encoded |
| 1230 CMVp-TFP250-pA | nucleotides 6436-7083 of | amino acids |
| Native | SEQ ID NO: 16 | 2146-2361 of |
| | | SEQ ID NO: 4 |
| 1232 CMVp-TFP250-pA | nucleotides 6444-7083 of | amino acids |
| Secreted | SEQ ID NO: 16 | 2149-2361 of |
| | | SEQ ID NO: 4 |
| 1234 CMVp-TFP250-pA | nucleotides 6448-7083 of | amino acids |
| Membrane | SEQ ID NO: 16 | 2150-2361 of |
| | | SEQ ID NO: 4 |
| 1231 MLP-TFP250-pA | nucleotides 6436-7083 of | amino acids |
| Native | SEQ ID NO: 16 | 2146-2361 of |
| | | SEQ ID NO: 4 |
| 1233 MLP-TFP250-pA | nucleotides 6444-7083 of | amino acids |
| Secreted | SEQ ID NO: 16 | 2149-2361 of |
| | | SEQ ID NO: 4 |
| 1235 MLP-TFP250-pA | nucleotides 6448-7083 of | amino acids |
| Membrane | SEQ ID NO: 16 | 2150-2361 of |
| | | SEQ ID NO: 4 |

In order to maximize the immune response in chickens using this vector system, each ORF encoding these two proteins was cloned separately into three FAV8 expression constructs leading to expression of either the native protein, a membrane anchored version of the protein and the secreted form of the antigen. The last two FAV8 constructs were achieved by fusion of an appropriate signal sequence upstream to the ORF of interest. The use of the FAV8 as a delivery vector provides a final product that has the potential to be introduced to the much larger broiler markets, because the vaccine will be able to be administrated inexpensively on a large scale.

Surprisingly, it was found that when full length r56 was cloned into an FAV8 vector it was unstable in the vector and as such could not provide appropriate protective immunity when delivered as a subunit vaccine. However, when truncated versions of the antigen were used, immunity was clearly demonstrated.

Three versions of r56 and three versions of TFP250 were produced. Version 1 is the native ORF supplied by UTS and unmodified apart from the insertion of its coding region into the FAV expression cassette. Version 2 is the addition of a signal sequence so the r56 or TFP250 is secreted from the cell. Version 3 also has a signal sequence attached but this is to direct r56 or TFP250 to the cell membrane.

The following Table summarizes the results of the first studies:

| | | ^{Ω}PCR | ^{#}SEQUENCE | |
|---|---|---|---|---|
| | *RECOMBINANT | CONFIRMATION | CONFIRMATION | ^{•}CONFIRMATION |
| | FAV8 | OF INSERTED | OF INSERTED | OF PROTEIN |
| | PRODUCED | DNA | DNA | EXPRESSION |
| r56-V1 | + | + | + | + |
| (native) | | | | |
| r56-V2 | + | + | + | + |
| (secreted) | | | | |
| r56-V3 | + | + | + | + |
| (membrane) | | | | |
| TF250-V1 | + | + | + | + |
| (native) | | | | |
| TF250-V2 | + | + | + | + |
| (secreted) | | | | |
| TF250-V3 | + | + | + | − |
| (membrane) | | | | |

For confirmation that a recombinant virus was obtained the virus was isolated and plaque purified using conventional means. In addition, PCR was used to confirm amplification of the entire inserted r56 or TFP250 DNA and polyA isolated from recombinant FAV DNA as well as each section (promoter, r56 or TFP250 and polyA). DNA isolated from recombinant FAV containing the entire inserted expression cassette consisting of promoter, r56 or TFP250 DNA and polyA also was sequenced in both directions to confirm fidelity of sequence insert. Protein expression was confirmed using anti-r56 or anti-TFP250 sera.

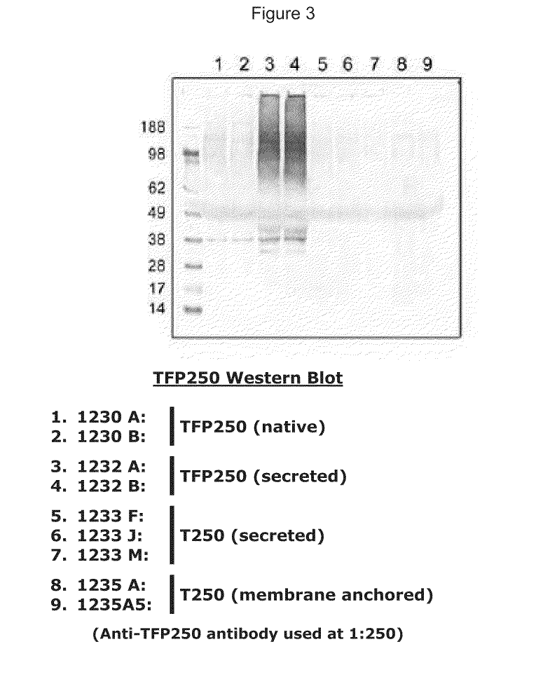

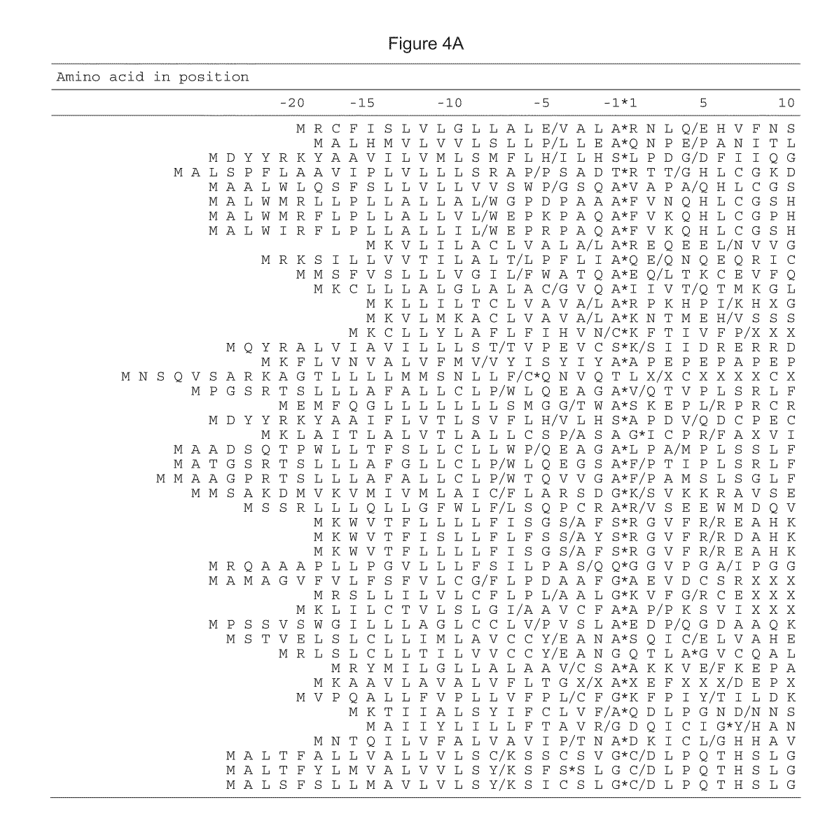

FIGS. 2 and 3 show protein analysis of r56 and TFP250 from the various constructs. Protein expression experiments were performed by infection of LMH cell lines with the different FAV8 constructs. These constructs were designed with all the protein expression versions (native, secreted and membrane anchor) and under control of different promoters (MLP, or CMVp). As negative controls, uninfected LMH cells and cells that were infected with the FAV8 vector alone were used. The r56 antigen was detected using polyclonal antisera raised to the recombinant 56 and the TFP250 was detected using mouse antisera to the recombinant peptide. The protein bands appeared at their appropriate molecular weight based on the expected size of the peptides predicted from the partial gene fragments that were cloned. The smearing seen in lanes 3 and 4 for the recombinant TFP250 (FIG. 3) was likely due to aggregation of the peptide with host cell material.

From the results, all of the constructs appeared to be well expressed apart from the TFP250 membrane anchor construct, which didn't show a clear band. This may be due to membrane anchoring of the expressed protein and potential conformational change after extraction from the membrane.

There are many different possibilities available to someone skilled in the art. To make the necessary constructs, three examples are below. In FIG. 5, there is depicted a scheme for the chemical synthesis of the restriction enzyme site, signal sequence in frame with an ORF of interest (for example a truncated r56, 82 kDa protein or TFP 250) and a second restriction enzyme site for directing the insertion of the construct into a FAV right hand end expression cassette.

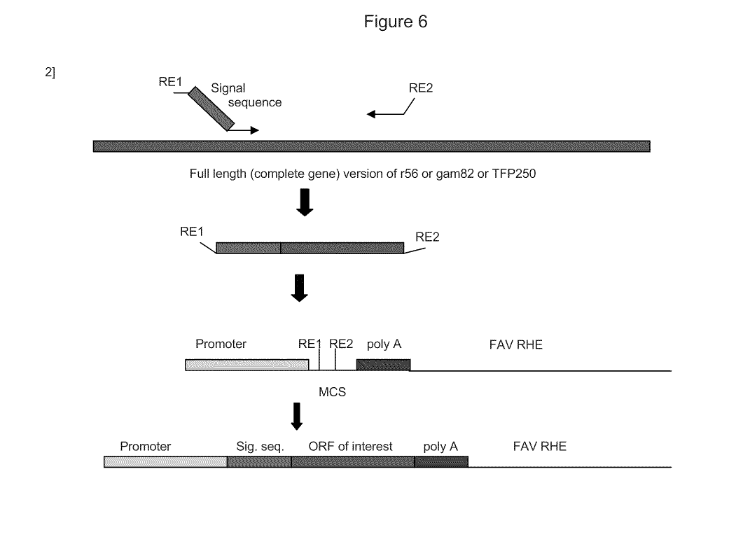

Alternatively, (FIG. 6) the skilled person may PCR amplify the desired sequence from ORF of interest using primers that have the appropriate RE sites as well as a signal sequence in frame.

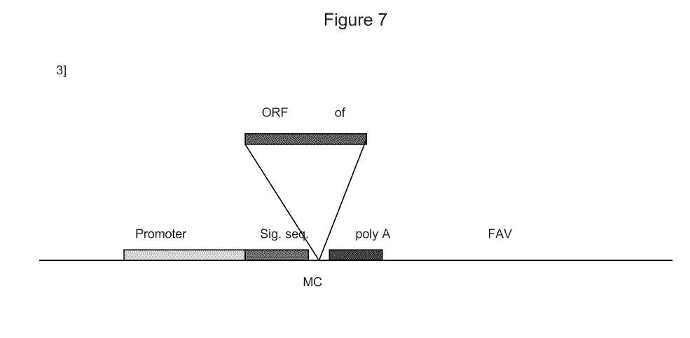

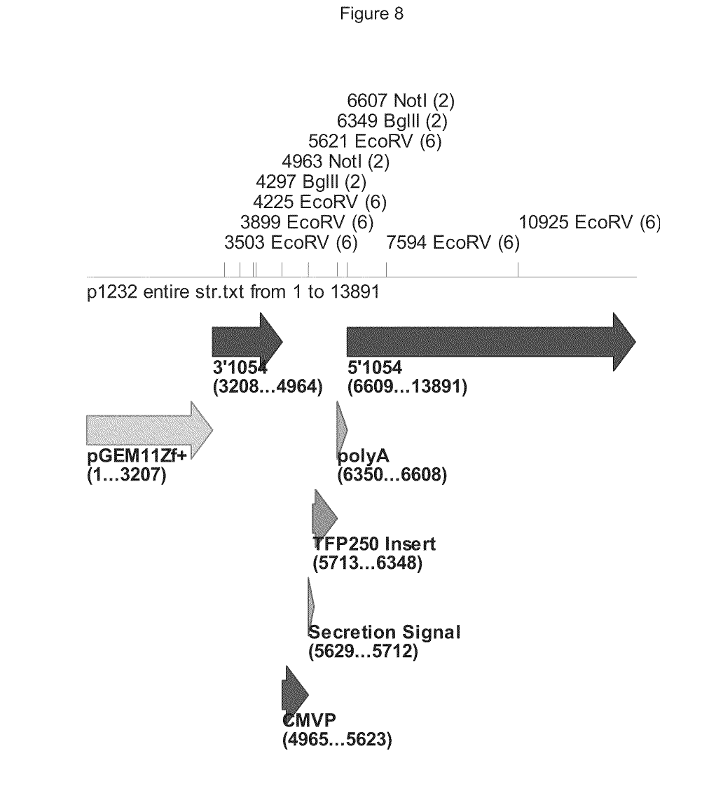

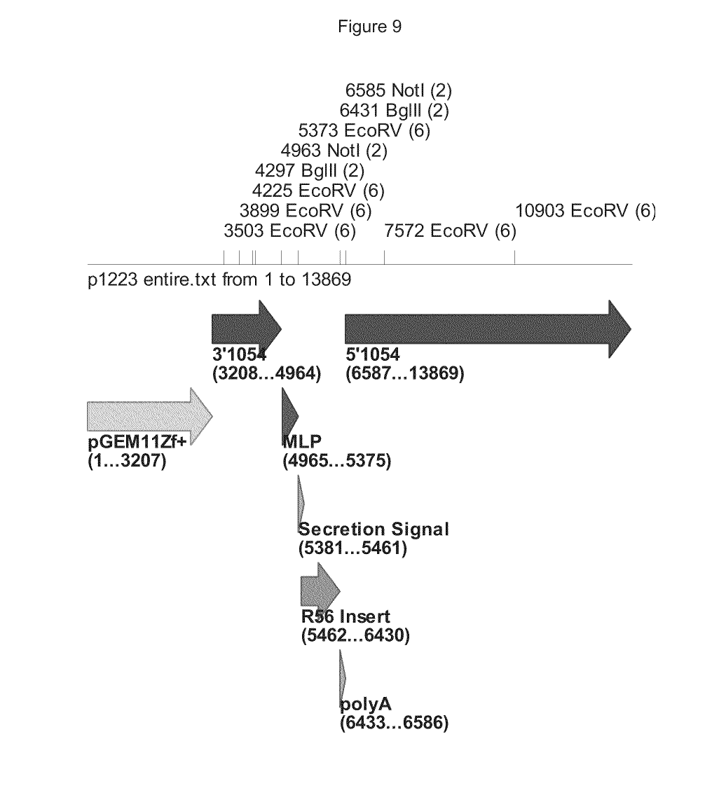

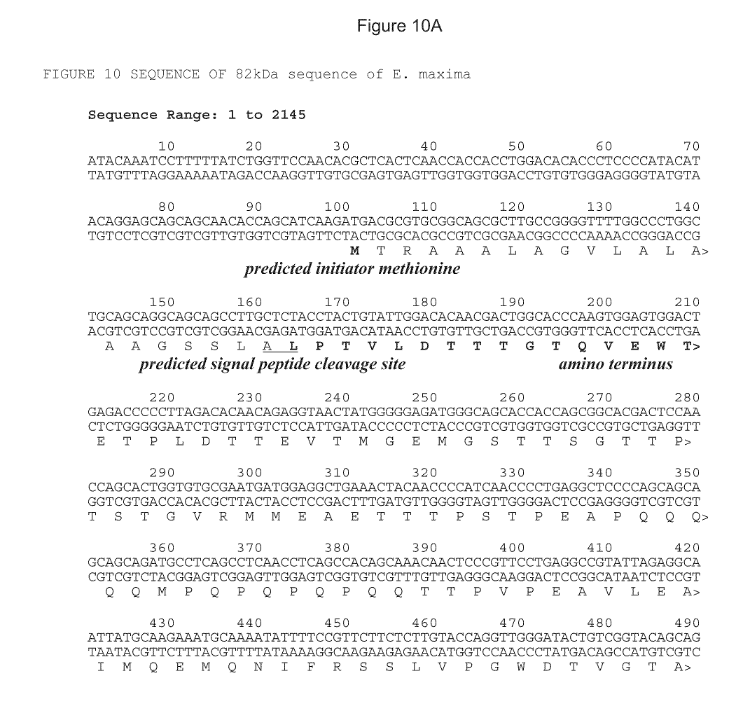

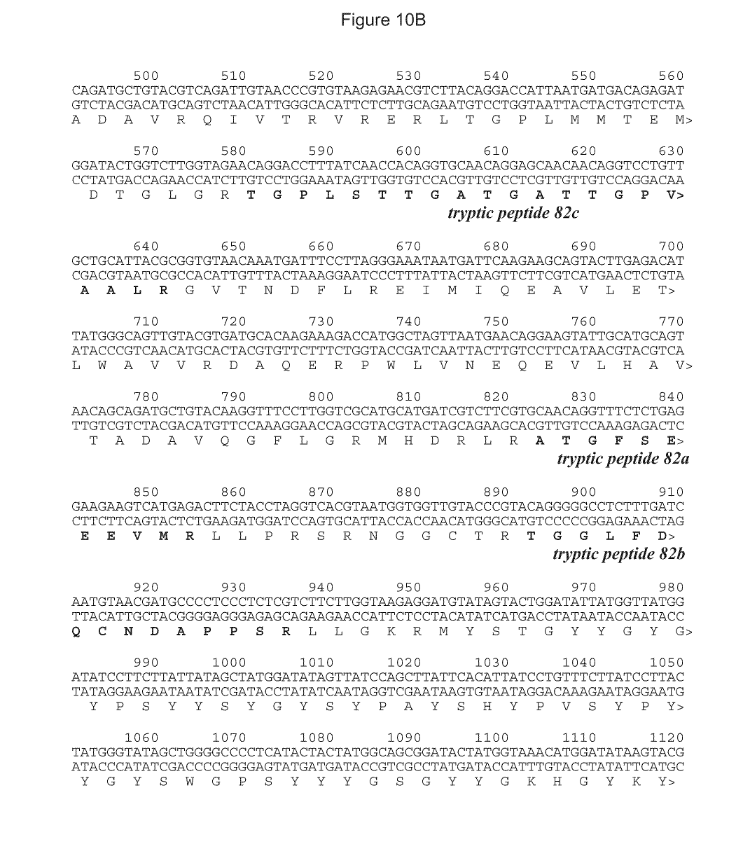

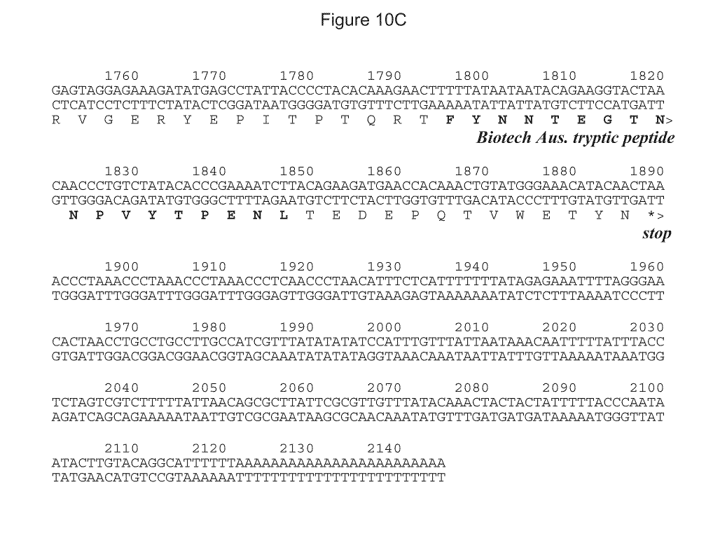

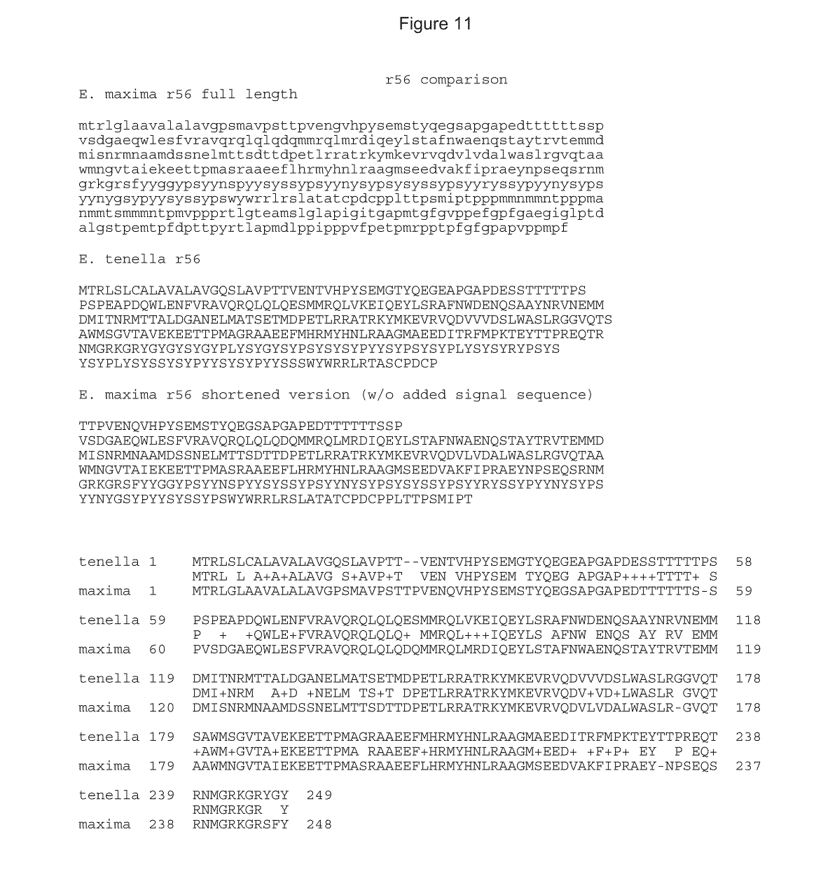

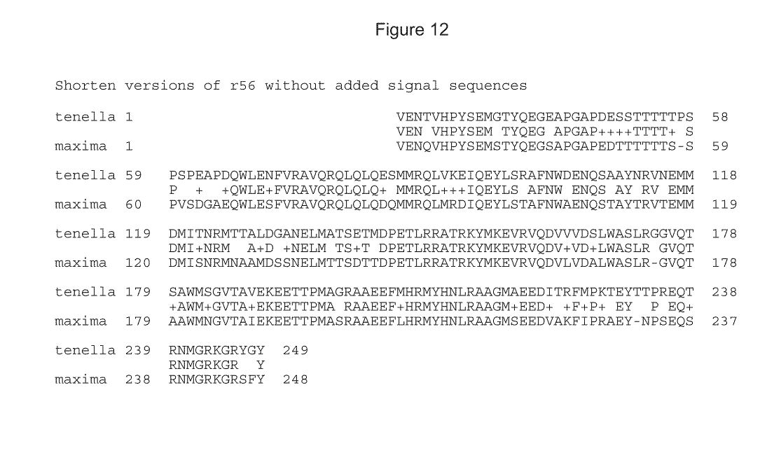

In other examples, the skilled person may construct the FAV RHE Expression cassette to contain the promoter with a signal sequence then a MCS for insertion of the ORF of interest in frame with the signal sequence (FIG. 7).

### Example 2

### Induction of Protective Immune Response

The present example tests the ability of the FAV8-r56 and FAV8-TFP250 vectors to induce a protective immune response that blocks E. maxima development and prevents parasite lesions.

TABLE 2

| Group | Treatment |
|---|---|
| A | Non-vaccinated |
| B | 20,000 Eimeria maxima oocysts per chicken. |
| C | 40,000 Eimeria maxima oocysts per chicken. |
| D | 80,000 Eimeria maxima oocysts per chicken. |

In order to evaluate the number of oocysts needed for producing lesions, a pre-trial is conducted in which 30 SPF chickens are raised under coccidiosis free conditions (cleanness, water and feed) in portable, clean cages. The chickens are treated weekly to prevent coccidial infection prior to challenge by administering amprolium through their drinking water. On day 28 chickens are challenged with the appropriate number of Eimeria maxima oocysts per os (see Table 2). On day 34 lesion scoring (at least five chickens per dosage level) is performed. The group that has the most consistent lesion scores with an average of 3-4, is chosen for the high challenge dosage used in the trial. The same batch of oocysts used for the pretrial are stored in 2% potassium dichromate at 4° C. and used in the trial.

The pretrial is then conducted on 400 SPF chickens obtained from the hatchery near Amidale (allows for up to 10% mortality in the first 3 weeks) as well as 180 fertile eggs at 18 days of incubation for in ovo immunization.

The following vaccines and controls will be used:

Controls: Unvaccinated (negative control); FAV8 vector alone (negative control); r56 protein vaccination; and TFP250 protein vaccination. The vaccines to be tested are FAV8 construct based vaccines as follows: FAV8—r56 native protein; FAV8—r56 N-terminal membrane anchor; FAV8—r56 secreted form; FAV8—TFP250 native protein; FAV8—TFP250 N-terminal membrane anchor and FAV8—TFP250 secreted form.

The titer of the FAV8 constructs will be 1×10⁸ per dose. The vaccine is to be administered by oral, in ovo, or subcutaneous vaccination as described below.

For oral vaccine administration a 1-ml syringe connected to a 21-gauge blunt-tip needle is used. The bird is held upright and gently its mouth is opened and the blunt-tip needle is inserted into the anterior portion of the choanal cleft. The vaccine is administered slowly allowing the vaccine to bathe the choanal cleft and oropharynx prior to being swallowed. In this manner, most of the nasopharynx and almost the entire oral cavity come into contact with the vaccine as it is being administered.

For in ovo vaccination at 18 days incubation, 180 eggs receive into the allantoic fluid an injection of virus (groups 5,6,17,18,23 & 24 eggs each see Table 3 below) at the same dose level as that used for the day old chicks.

For subcutaneous and intramuscular vaccination using the recombinant antigens bacteria will be concentrated 10 fold of the fermentation concentration, lysed by sonication in urea buffer: 25 mM Tris pH 8.0, 6M urea, 100 mM NaCl and filtered through a 0.8 μM filter. Emulsions (25% water phase) of 100 ml will be prepared from each 25 ml CE sample and from Urea buffer and PBS. Freund's complete adjuvant is used for the trial and the emulsion is prepared just prior to injection.

Each chicken will be vaccinated first subcutaneously with 0.5 ml per dose on day 1 and then boosted intramuscularly with 0.5 ml per dose on day 14.

The following Table summarises the experimental design for vaccination.

| | | No. of | |
|---|---|---|---|
| Group no. | Test type | chicks** | vaccine |
| 1 | Oocyst counting | 20 | Unvaccinated (negative control). |
| 2 | Lesion scoring | 20 | Unvaccinated (negative control). |
| 3 | Oocyst counting | 20 | PBS in Freund's adjuvant (negative control group) |
| 4 | Lesion scoring | 20 | PBS in Freund's adjuvant (negative control group) |
| 5* | Oocyst counting | 20 | FAV8 vector alone (negative control) In ovo vaccination |
| 6* | Lesion scoring | 20 | FAV8 vector alone (negative control) In ovo vaccination |
| 7 | Oocyst counting | 20 | r56 vaccination in Freund's adjuvant |
| 8 | Lesion scoring | 20 | r56 vaccination in Freund's adjuvant |
| 9 | Oocyst counting | 20 | TFP250 vaccination in Freund's adjuvant |
| 10 | Lesion scoring | 20 | TFP250 vaccination in Freund's adjuvant |
| 11 | Oocyst counting | 20 | FAV8 - r56 native protein |
| 12 | Lesion scoring | 20 | FAV8 - r56 native protein |
| 13 | Oocyst counting | 20 | FAV8 - r56 N-terminal membrane anchor. |
| 14 | Lesion scoring | 20 | FAV8 - r56 N-terminal membrane anchor |
| 15 | Oocyst counting | 20 | FAV8 - r56 secreted form |
| 16 | Lesion scoring | 20 | FAV8 - r56 secreted form. |
| 17* | Oocyst counting | 20 | FAV8 - r56 secreted form In ovo vaccination |
| 18* | Lesion scoring | 20 | FAV8 - r56 secreted form In ovo vaccination |
| 19 | Oocyst counting | 20 | FAV8 - EmTFP250 native protein. |
| 20 | Lesion scoring | 20 | FAV8 - EmTFP250 native protein. |
| 21 | Oocyst counting | 20 | FAV8 - EmTFP250 membrane form |
| 22 | Lesion scoring | 20 | FAV8 - EmTFP250 membrane form |
| 23* | Oocyst counting | 20 | FAV8 - EmTFP250 secreted form In ovo vaccination |
| 24* | Lesion scoring | 20 | FAV8 - EmTFP250 secreted form In ovo vaccination |

In order to avoid coccidiosis contaminations the chickens will be treated weekly with amprollium in their drinking water on days 7, 14 and 21. Moreover, coccidial infection will be avoided by thoroughly cleaning the isolators, facility, etc. and all workers will change clothing upon entry. On day 26, fecal samples will be taken to test for the presence of Eimeria oocysts. On day 28, chickens used to measure oocyst excretion will be challenged with 100 E. maxima sporulated oocysts per os and those for lesion scoring will receive the number of sporulated oocysts that cause significant pathology as determined in a pretrial (20-50,000).

The above trial will follow the following schedule.

On day 18 of egg incubation, 1st in ovo vaccination of group 3, 9 and 12.

On day 21 of egg incubation, the chicks from in ovo vaccinated groups are hatched in 3 separate egg incubators.

On day 1, 1st vaccination of all of the other groups is undertaken.

On day 14, 2nd vaccination of all chicks (including groups 3, 9 and 12) is undertaken.

On day 28, the chickens are bled for serological tests and then challenged with E. maxima, with the right number of oocysts required per chicken for lesion scoring (based on results from the pretrial) or 100 oocysts per chicken for oocysts counting.

On day 34 lesion scoring is performed on groups that received the large dosage of oocysts.

On days 34-37 feces is collected in a single pool from each group of chicks infected with 100 oocysts, and on day 37 oocyst counts are performed on all samples.

On day 42 (14 days post infection) the birds are bled for serological tests and sacrifice.

In vitro testing for evaluation of immune response is performed in which four weeks post immunization (day 28) serology test is conducted on FAV8 coated plate (TropBio Ltd.) to ensure FAV8 constructs infection (for all the flock). ELISA assays also will be performed four week post immunization sera as well as 14 day post challenge sera, on APGA, r56 and TFP250 coated plates (for all the groups). Further, protein analysis will be performed using Western blots with preparative gametocyte and recombinant TFP 250 containing blots using anti-r56/APGA/anti-TFP250/as positive control sera as well as normal chicken serum as a negative control.

In an initial trial conduced as set out above, it was found that the vaccinated groups with the highest protection were the r56 secreted group and r56 native group with average lesion scores of 1.31 and 1.36 respectively, where a score of 1 is considered acceptable for obtaining good performance results in broiler chickens. The difference between an average lesion score of 2.37 in the control group to 1.31 in the vaccinated group can definitely be assigned the gap between a diseased chicken to a protected chicken.

It was concluded that the FAV8 vector containing the r56 secreted construct worked very well in inducing protection in one day old chicks based on both lesion scoring and oocyst counts. Groups that were vaccinated with this construct by in ovo immunization did not show such a high level of protection. It is believed that the reason for this is that the chicks did not have enough time to be exposed to the virus due to the early hatch. Nevertheless, in ovo vaccination remains a viable and economical approach for future vaccinations. Oral vaccination of one day old chicks has proven effective and as such it is expected that spraying or feeding vaccines to such birds will be a useful method of providing long term immunity to broilers.

In addition to providing good results with the r56 constructs, it was found that both by lesion scoring and oocyst counts the TFP250 secreted construct also induced a significant (albeit lower 20-30%) level of protective immunity.

1. A coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, a multiple cloning site for insertion of an open reading frame (ORF) to allow insertion of an ORF in frame with said hydrophobic signal sequence, a polyadenylation signal; and an avian adenovirus genome.

2. The coccidiosis vaccine of claim 1, wherein the ORF of interest encodes an antigen selected from the group consisting of a truncated r56 antigen of Eimeria maxima, a truncated TFP250 antigen of Eimeria maxima and a truncated 82 kDa antigen of Eimeria maxima

3. The coccidiosis vaccine of claim 1, wherein the multiple cloning site contains an ORF that encodes a truncated r56 antigen of Eimeria maxima, in combination with a truncated TFP250 antigen of Eimeria maxima and/or a truncated 82 kDa antigen of Eimeria maxima.

4. The coccidiosis vaccine of claim 1, wherein said avian adenovirus genome is selected from the group consisting of the genome of FAV 1, FAV 2, FAV 3, FAV 4, FAV 5, FAV 6, FAV 7, FAV 8, FAV 9, FAV 10, FAV 11 and FAV 12.

5. The coccidiosis vaccine of claim 1, wherein said avian adenovirus genome is an FAV 8 genome.

6. The coccidiosis vaccine of claim 1, wherein said recombinant avian adenovirus vector further comprises a cleavage sequence immediately upstream of the cloning site for the insertion of the ORF of interest, wherein expression product from said vector produces a soluble product.

7. The coccidiosis vaccine of claim 2, wherein said nucleic acid that encodes an antigen selected from the group consisting of:
a) a truncated R56 comprises the sequence of nucleotides 70-1035 of the full length r56 sequence shown in SEQ ID NO:13 but does not encode the complete r56 protein sequence shown in SEQ ID NO:2;
b) a truncated r56 encodes the truncated R56 fragment that consists of amino acids 24-345 of SEQ ID NO:2 or a fragment of amino acids 24-345 of SEQ ID NO:2;
c) a truncated TFP250 comprises the sequence of nucleotides 6448-7083 of the full length TFP250 sequence shown in SEQ ID NO:16 but does not encode the complete TFP250 protein sequence shown in SEQ ID NO:4; and
d) a truncated TFP250 consists of the nucleic acid sequence of nucleotides 6448-7083 of SEQ ID NO:16

8. A coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated r56 that consists of amino acids 24-345 of SEQ ID NO:2 or a fragment of amino acids 24-345 of SEQ ID NO:2 inserted in frame with said hydrophobic signal sequence, a polyadenylation signal; and an avian adenovirus genome.

9. A coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated TFP250 that consists of the nucleic acid sequence of nucleotides 6448-7083 of SEQ ID NO:16 inserted in frame with said hydrophobic signal sequence, a polyadenylation signal; and an avian adenovirus genome.

10. A coccidiosis vaccine for the protection of poultry against Eimeria infection, said vaccine comprising a recombinant avian adenovirus vector comprising a promoter operably linked to a hydrophobic signal sequence comprising a nucleic acid that encodes a membrane anchoring domain, and a nucleic acid that encodes a truncated 82 kDa antigen of Eimeria maxima inserted in frame with said hydrophobic signal sequence, a polyadenylation signal; and an avian adenovirus genome.

11. A multivalent coccidiosis vaccine preparation that comprises a coccidiosis vaccine of claim 7 and
a coccidiosis vaccine of comprising a recombinant avian adenovirus vector comprising a promoter operably linked to a hydrophobic signal sequence comprising a nucleic acid that encodes a membrane anchoring domain, and a nucleic acid that encodes a truncated TFP250 that consists of the nucleic acid sequence of nucleotides 6448-7083 of SEQ ID NO:16 inserted in frame with said hydrophobic signal sequence, a polyadenylation signal; and an avian adenovirus genome; and/or
a coccidiosis vaccine comprising a recombinant avian adenovirus vector comprising a promoter operably linked to a hydrophobic signal sequence comprising a nucleic acid that encodes a membrane anchoring domain, and a nucleic acid that encodes a truncated 82 kDa antigen of Eimeria maxima inserted in frame with said hydrophobic signal sequence, a polyadenylation signal; and an avian adenovirus genome.

12. The multivalent coccidiosis vaccine preparation of claim 11, further comprising an immunogen selected from the group of consisting of Marek's Disease virus (MDV), Newcastle Disease virus (NDV), Infectious Bronchitis virus (IBV), Chicken Anaemia Virus (CAV), Infectious bursal disease virus (IBDV), Avian influenza (AI), Reo virus, Avian Retro virus, Fowl Adeno virus, Turkey Rhinotracheitis virus, Salmonella spp. and E. coli.

13. A method of immunizing a subject against infection by Eimeria tenella, Eimeria maxima, Eimeria acervuline, Eimeria necatrix, Eimeria praecox, Eimeria mitis or Eimeria brunetti, comprising the step of administering to the subject a vaccine of claim 1.

14. The method of claim 13, wherein said administering elicits an enhanced level of immunity as compared to immunity seen when said subject is immunized with an FAV vector comprising a full length r56 or a full length TFP 250 antigen or a full length 82 kDa antigen.

15. The method of claim 13, wherein said subject is of an avian species selected from the group consisting of chickens, turkeys, geese, ducks, bantams, quail and pigeons.

16. The method of claim 15, wherein said avian species is chickens.

17. The method of claim 16, wherein said chickens are adult broiler chickens.

18. The method of claim 13, wherein said administering comprises spraying said subject with said vaccine, feeding said subject said vaccine in food, and providing said vaccine in the drinking supply of said subject.

19. A combination vaccination therapy for providing protective immunity against Eimeria tenella, Eimeria maxima, Eimeria acervulina, Eimeria necatrix, Eimeria praecox, Eimeria mitis or Eimeria brunetti, to a chicken population comprising the step of administering to the subject a vaccine of claim 1 and administering CoxAbic® to said chicken population.

20. The combination vaccination therapy of claim 19, wherein said CoxAbic® is administered to breeder hens to confer immunity to chicks upon hatch and said vaccine of claim 1 is administered to the chicks at day 1 after hatching and later and to adult broiler hens of said population.

21. A recombinant avian adenovirus vector comprising an avian adenovirus genome comprising a heterologous promoter, an heterologous hydrophobic signal sequence, a multiple cloning site, and a polyadenylation sequence, wherein said promoter and said hydrophobic signal sequence are located upstream of a multiple cloning site, wherein insertion of a ORF of interest into said multiple cloning site will result in an expression vector capable of expressing said ORF of interest under the control of said promoter and in frame with said signal sequence.

22. The recombinant avian adenovirus vector of claim 21, wherein said hydrophobic signal sequence comprises a cleavage site to allow secretion of the expression product of said ORF of interest from host cell in which it is expressed.

23. The recombinant avian advenovirus vector of claim 21, wherein said signal sequence does not contain a cleavage site thereby resulting in expression of a fused expression product of said ORF of interest being anchored to the cell surface of the host cell.

24. A recombinant avian adenovirus vector comprising a promoter operably linked to a hydrophobic signal sequence comprising a nucleic acid that encodes a membrane anchoring domain, a multiple cloning site for insertion of a ORF of interest to allow insertion of a ORF of interest in frame with said hydrophobic signal sequence, a polyadenylation signal; and an avian adenovirus genome.

25. The recombinant avian adenovirus vector of claim 24, further comprising a cleavage sequence immediately upstream of the cloning site for the insertion of the ORF of interest, wherein expression product from said vector produces a soluble ORF product.

26. A recombinant avian adenovirus vector comprising a promoter operably linked to
a) a hydrophobic secretion signal sequence and cleavage site or a signal sequence comprising a nucleic acid that encodes a membrane anchoring domain,
b) a nucleic acid that encodes a truncated r56 protein of Eimeria maxima,
c) a polyadenylation signal and
d) an avian adenovirus genome.

27. A recombinant avian adenovirus vector comprising a promoter operably linked to
a) a hydrophobic secretion signal sequence and cleavage site, or a signal sequence comprising a nucleic acid that encodes a membrane anchoring domain
b) a nucleic acid that encodes a truncated TFP250 protein of Eimeria maxima,
c) a polyadenylation signal and
d) an avian adenovirus genome.

28. A recombinant avian adenovirus vector comprising a promoter operably linked to
a) a hydrophobic secretion signal sequence and cleavage site, or a signal sequence comprising a nucleic acid that encodes a membrane anchoring domain
b) a nucleic acid that encodes a truncated 82 kDa protein of Eimeria maxima,
c) a polyadenylation signal and
d) an avian adenovirus genome.

29. The recombinant avian adenovirus vector of claim 26, wherein said nucleic acid that encodes a truncated r56 comprises the sequence of nucleotides 70-1035 of the full length r56 sequence shown in SEQ ID NO:14 but does not encode the complete r56 protein sequence shown in SEQ ID NO:2.

30. The recombinant avian adenovirus of claim 26, wherein said nucleic acid that encodes a truncated r56 encodes the truncated R56 fragment that consists of amino acids 24-345 of SEQ ID NO:2 or a fragment of amino acids 24-345 of SEQ ID NO:2.

31. The recombinant avian adenovirus vector of claim 26, wherein said nucleic acid that encodes a truncated TFP250 comprises the sequence of nucleotides 6448-7083 of the full length TFP250 sequence shown in SEQ ID NO:16 but does not encode the complete TFP250 protein sequence shown in SEQ ID NO:4.

32. The recombinant avian adenovirus vector of claim 26, wherein said nucleic acid that encodes a truncated r56 encodes the truncated r56 fragment that consists of amino acids 2150-2361 of SEQ ID NO:4.

33. The recombinant avian adenovirus vector of claim 27, wherein said nucleic acid that encodes a truncated TFP250 comprises the sequence of nucleotides 6444-7083 of the full length TFP250 sequence shown in SEQ ID NO:16 but does not encode the complete TFP250 protein sequence shown in SEQ ID NO:4.

34. The recombinant avian adenovirus vector of claim 27, wherein said nucleic acid that encodes a truncated TFP250 encodes the truncated TFP250 fragment that consists of amino acids 2149-2361 of SEQ ID NO:4.

35. The recombinant avian adenovirus vector of claim 26, wherein said secretion signal sequence is selected from the group consisting of the secretion signal sequence of chicken gamma interferon, porcine gamma interferon, and Human Influenza H1N2.

36. The recombinant avian adenovirus vector of claim 26, wherein said membrane anchoring signal sequence is selected from the group consisting of the secretion signal sequence of an avian influenza HA antigen.

37. A vaccine comprising a recombinant avian adenovirus vector of claim 26.

38. A method of eliciting an immune response in an avian population comprising administering to said population a vaccine of claim 38.

39. A method of vaccinating a fowl population against coccidiosis comprising administering a vaccine comprising a recombinant avian adenovirus vector of claim 24, wherein administration of said vaccine elicits an increased immune response as compared to administration of a vaccine comprising full length r56 or full length TFP250.

40. An isolated cell comprising recombinant avian adenovirus vector of claim 24.

41. A pharmaceutical formulation comprising a recombinant avian adenovirus vector claim 24 and a suitable excipient.


SEQUENCE LISTING (124 sequences)

SEQ ID NO: 1 - DNA, 1754 bp, Eimeria maxima
agcagaacatagggagttcatctgttccttcttttcatcatttattcctcgtttctcaccgttttattttttttgtgtaaccctctccgctgttgagtcccaatgacccgcctcggcctcgctgctgtcgcgctggctctcgccgtgggcccttccatggcagtgcccagcaccactcctgttgagaaccaggttcacccttacagcgagatgagtacctaccaggaggggagtgccccgggggctccggaggacaccaccaccaccactacgtcgtcccctgtttccgatggagccgagcagtggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccatggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaactccccccccaatgatgaacatgatgaacaccccaccccccatggcaaacatgatgaccagcatgatgatgaacactcccatggttcctcctccccgcaccctcggaactgaagccatgagcctcggcttggcccccatcggtatcaccggcgcccccatgacaggtttcggtgttcctcctgagttcggtccctttggagccgaaggtatcggcctccccaccgatgccctcggcagcacccccgaaatgacaccattcgacccaactaccccctacagaactctcgcccccatggacctcccccccatcccccctcctgtcttccctgaaacccctatgaggccacctactcccttcggcttcggacctgcacctgttcctcccatgcccttctaaacgacctaccatccctcaatccatagctcacatttcgtagcctcaaaacagttttttgttcatttcacttccaggactcatgctgcgacatttgcattcgtacctcgaaaccgtcaacctcaaaccccaaaccattctgtgacctcccctcgcaaacgcggaaggcggaacattttttctgaagtatattactacgttaaaaaaaaaaaaaaaaaaaaaaa

SEQ ID NO: 2 - protein, 476 aa, Eimeria maxima
MTRLGLAAVALALAVGPSMAVPSTTPVENQVHPYSEMSTYQEGSAPGAPEDTTTTTTSSPVSDGAEQWLESFVRAVQRQLQLQDQMMRQLMRDIQEYLSTAFNWAENQSTAYTRVTEMMDMISNRMNAAMDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFYYGGYPSYYNSPYYSYSSYPSYYNYSYPSYSYSSYPSYYRYSSYPYYNYSYPSYYNYGSYPYYSYSSYPSWYWRRLRSLATATCPDCPPLTTPSMIPTPPPMMNMMNTPPPMANMMTSMMMNTPMVPPPRTLGTEAMSLGLAPIGITGAPMTGFGVPPEFGPFGAEGIGLPTDALGSTPEMTPFDPTTPYRTLAPMDLPPIPPPVFPETPMRPPTPFGFGPAPVPPMPF

SEQ ID NO: 3 - DNA, 7990 bp, Eimeria maxima
caacatttcttcttcctttttcttcttcgagcttctttagctcgattttctggcccttgcagctctccgcgggtgcagggcgcagccagctcactactgcctttcacagcgtcgttccccaccttggcccatgtgccacatggtcatttttcttcagtttgttcatgagaagagctgctacagtgtagctcgaactcaactttaaacgcagccgtttcagcggcgacaatatgctgcatcgcaacccgcggtgggcgctttgtgcagccctcgctgcactctatggcggaacaggaatcgccagcgccgaagttaacaatgaattgagcaagtgcgaatctgggtggacaccctggactacctgcaacccgcaaactggtctgcgggagaggcacaatgcacagtgcgagacatgggtggaggttgaggaatgccagaagctgacaggatgtggcaactggactccttggtctcccggcgatatgtcgtgtgtggtgggacagtttcaaacccgcaacagggagggctgcccagaggtgcaggaagtgagggcatgcaggcctgtacttctagaatgcaacgatcaatggaccccctggacaatgtgcgacaccaaccgcgtccaggaaagatacaactcaaagtgcggacccgtcgaagtccgcgagtgcaacatggacgacgcagagatcgagaaatgcggcgagttcgtggaatgggatccccctatgaatggagactgcgtacgcgggggtacccacacgcgttaccgtcaaaactgcccagaccgcaaagaggtgcgggtgtgcggagcctttgattgcagtagctgctctgtaaacgccacttgcgatcccattggtgcatcctgcgaatgcaagcctggtttccgcggcaatgggaagacctgcgaggccttcaacccctgcgaagatacccctgcaccttgcgacagcaacgccatctgcaccccagacggcaatgacgccaaatgccagtgcaaggcaggctgggacgcagattccggagcaggcagcagcaagaagccttgcgttgaggtcgacgagtgcgcatccaacacccaccagtgcccggcacactccacatgcatcaacaccaagggctcttataagtgcgactgcaaccagggatacgtcaagggagaggacggacagtgtcatgacgtcgatgaatgcaccaacggagagcacacctgccccgctcactccacttgtttgaatacagctggcagctacgagtgccgctgcgacactgggtacagcggaaatgcaactgcagacagcccttgcaagaacattgacgaatgcgccaaccccaacgcctgctcggccaacgctatctgcacagacaccgacggctccttcacctgcagctgccccgaagggtacagcggccagggaacccatgactctccctgctccaagatcgacttctgcgcagacccctcactcaatacatgcggagcccactccacttgcgtgaacaccctcacatctttcaagtgcatctgcgatgcgggatatgaaggcgccggcactcgcgagagcccgtgcgtggacgtgaacgagtgctcgaacgagaagcccacaaacaactgcaacagaaacgcaaactgcaccaacaccgagggatcctacacttgcgaatgcaagcccggtttctctggcgacggcatgggtcccaacgggtgtaccgacatcgacgagtgcgcggcggagcagtccccctgcgaccctcacgcctcctgcagcaacactgagggctcgtatgtatgcacctgcaacaccggctacgagccagcttcaaccgacgggcatgcatgcaaagatatcgacgagtgcgccaccggtgcagctgggtgccacgtgtcagcacagtgtctgaacacggacggcagctacgagtgcaagtgtcttgagggcttcgtcggcgacggaaagacctgcaacgacgtcgatgagtgcgctgcggcgacatctccttgcggtgacaacactcactgccagaacacaattggcagctacgagtgcgagtgcaaggctggctatggcaacatgcaagacaacgcatgcagcgacattgacgagtgcaaggatgcgaacaccaagatccctgacaactgtctttgcgtgaacaatgatggcagctactcccttgaggcgaaggctggatacgaattggtgaacggcgagtgcatcaagatcgacttctgcgcccgcggcgcatgcaactcgctggcctcctgcaaggagaatgaagaaggcacagcggcgatctgcacctgcctgccaggctacagcggcgacggcactgctgaaggccactgcaacgacattgacgagtgtgcaggtcagaatgactgtgctcctgccgagcagggaggcatctgcgagaacactgtcggctcgtacacctgcaagtgcaaagaggggtacaggcaagatggaaactcatgcactgagatcgacgagtgcgctgagggaacccacaactgccacccttccgccacctgcagcaacacccccggaagcttcacctgccaatgcaacagtggattcactggcagcggtgtggagtgcgaagacattgacgagtgctcaactgaggcagatgattgtggtgcaaacaccatctgcagcaacaccattggtgctttcgagtgcaactgccgtgaaggctatgaacgcgcagacgcaaagacgtgcgtcgacatcgacgaatgcgcgacaggcacacacacttgctcgaaccacgccacctgcaccaataccgatgggtcattcacatgccagtgcaaccccggcttcgaaggtgacggccacaagtgcgaggacatcgacttctgcggtgctggacagcacgactgcaatgtgcatgccgagtgctctgagagcgaggacaacaccactttcaagtgcacctgtataacagggtacgctggagacggccatggcgaggcaggctgccaagacattgatgagtgcgcagaagaaaacatctgcggaagcaacgctgtctgcacaaacaccgcaggaagctaccaatgcgcatgccgtgagggcttcgttgcatcagctgaacagcagcagcagggaaccccagcactggtttgcgtggacgtcgacgagtgcagcgacgcttcgaagaacacatgtgccaagccagccgacggaggcatttgcacaaacactgaaggcagctacgaatgcgcttgcaagccaggctaccaaggtgacggccacagctgcgcagacatcaacgaatgcactgcacagggcacctgcggcgaacacacaacttgcaagaacacacccggatccttccagtgcgactgcgttgagggattcgagcgcgctgatgaacgcacctgccgtgacatcaacgagtgcgagacaggagcagtcgtgctgccaccgaactccacctgcgtcaacactgaaggcagctacgacttcgactgcgttgctgggtaccgccgcactgatggagcttgtgtgaagatcgacttctgcaaggagaagggatgcaacgcaaacgccacatgccgcgaaaacgatgccggcaccgaggccatctgcacttgcaaggaaggctatgaaggcagcggagaaggcgaagatggttgccagaacatcaatgagtgcgagagaggcgaaccctgcaaggacttcggcgaaggcggtgtttgcgtcgacacaccaggatcattcacttgcgagtgcgctgctggattcattcaacgccgctccgtttgccaagatgttgacgaatgtctcgacggaaagctgaacacctgcgctgccaccggaggcgtctgctccaacaccgtcggttccttcacctgctcgtgcgccagcggcttcgaaggcgatggccacacctgcaatgatgtcgacgaatgcgcaacagcacagcacacctgtgacccgaatgccacttgcgtcaacaccgaaggcagcttcgagtgccgctgcaatgccggattcgagggcgacggacacacctgcgcagacatcgacgaatgcgcagacccagccaaaaacacatgcgatacacacaagggtgtatgccaaaacaccacagggtcctacacctgcggctgcaagaccggattcagtcttgcagctgacggaagcacatgcgaaaacgtcgacgagtgcgcggcgggaactgcaaactgcaacgagcgaagcttctgtaaggacacagagggttcctaccaatgcgagtgcaagaacggctacaaggctgcaggagaggactgtgtggacgttgacgagtgcgaggctggcgtgcatggatgcagcgagcacgcaatctgcacaaatacagacggcagctactcctgcgaatgcatggagggataccagggagacggcaaggcttgcgagaagacagtcggcgtctgcgactccgctccctgcggtgcccacgccacctgcgagcctgcaggggacaactacacttgcacatgccacccaggctacgagatgcgcgaaggagcctgcgttgacatcgatgagtgcacagcaggcagcctcaactgcgaccctcatgccatttgcacaaacaccgacggctccttcacttgcgtctgtggcagcggctataccggccttggcacatcctgcgaagacatcgacgagtgcgcgggtaacgcagcaggctgcgacatccacgccgtctgcacgaacactcccggatcgttcaagtgcgagtgcaagagcggcttcgaaggcgatggcacgcaatgcacggagaaggtgttgctccccggacagattcactgcgaagcctggactgcatggacagagtgtaccgacggcgccaaaaccagcacacgcagctgccttgcactgccgcttaagaaggagatgcgcgcctgccctgcagctgacttctcccagtgcggagagttcactgaatggactgcctgccctggaaccaacaataacctgtctcataggcgcactgaaagattcggagaacccggatgcgaagatgcagaggaagtccgcgaatgcccagatgaagagaccgagcagaaatgcggcgcctggggtgagtggaccgcctgcggcgacccatcccctggcctgagaactcgcgcacgcgagaactgccccgatgtggtagagttcgagcgttgcactatgcccagtgagcctgaggctggcgaagtgactgagcctcacacagaaggaggagccggagttggtggcgaagtgactgagcctgacacggaagaaggagccggagttggtggtgaagtgcagcccggtacagaagaaggagcaggagttggtggtgaagtgcagcccggtacagaagaaggagccggagttggtggtgaagtgcagcccggtacagaagaaggagccggagttggtggtgaagtgcagcccggtacggaagaaggagccggcattggtggcgaagtgactgagcctgacaccgaaggaggagccggagttagtggcgaaccgaccgaagaagagggcaccgaaagcaccggtccatgcaaagagttcggaccctggacggcctgcaaggaggacgagaacggagtcggcatccaacgccgtatgtgcgccggcagagaagacatcatcgaatccagaatttgcactgtcacggatgactgcggagaatggaccccctggtcaacttgcactaacggcagccaggccagaaacaaacgcttctgcaccaacgttagggaagtccgtctctgcggagctgacattccagttacagacggatgcacgtggagcgagtggacttcttgcagtctagtcaatgaggagggcggctacttccgcacgcgcacatcctctgactgcaacatgaatgaagtgcaggcctgctctcccagcagcagcacaaccgcagacagcgaaacagaaggcacctgctctgcatggaacccctggacggagtgctcgaacggccaccagacacgcaagtgtgccacaatggaagcagaagaatcgcgcacttgcggagagactccagagaactgcggagaattcggccccttcgaacccgcaaactgcacggccggccaaatggtcaccaggacgcgcacctgcggagaaaccgagcagaaggaaaccaaactgtgcgacgtcagctccaccgaagaaggaaaacaatgcggtcagtggggcccatggagcgaatgcaacatccacctgggctcagaggacaatgtgcgtgttcgtgaggacaccgcttgcggcgtgacggagtacgaggagtgcagcaagccggcgaacaacgcctttgtctgcacaccttggagtgaatgctcggacaagaaggagcggagaacgtgcaccatccgcaaaaacggtcttgttcagacacgtcaagaattcagaacatgcagtgtagacatcgccacaacttgcggcgatttcggcgcatggtctgaatgcaacgctgagggcttgcatcagcgcagtctcgagaaatgccccgacgtcatcgaggtcgcaacttgcggcagtgaggattgcccgccattcggcgagtggactgaatgcggcgttccagaggagggcatgcgttctcgccaacgcattgactgcgttgaatctgcagcctgccagtgcacagaagtggagagctgcttcgacaccgaattgcaccccattccagcccccggtacggaaacaggcgaaggagagggagagaccgagacaggcgaaggcgaaactggtgaagcaggtggcgaggaaggcgagcaaacaggagaaggcgaagtgcagcccccagaagaagagcttcctggggagagtgtaactgagcctgaggagaagcctgaggaggagctacctgaggaggaggttactgagcctgaggagaagcctgaggagggtgtgactcagcctgaggagacacctgagcagcctgttgagggtaccgaagaagagggcaagcaggagtctgaggctgcccccgaaactcctgccgtccagccaaaaccagaggagggtcacgaacgcccagaacccgaagaggaggaggagaagaaggaagaaggcggcggcttcccaacagctgcagtggcaggaggtgttggtggtgtgttgctcatagctgctgtaggtggtggtgttgcagccttcactagcggcggaggtggcgctggcgcacaggaggcagaacaggtcgagttcgaaggagaagataccggagcagcaactgccgagacacctgaagccgatacagttatcgacatcacagacgaagacgactactgggccgacagcggcgacattcagtaaagttgaatgtctgttttcttccaaggagaagatacaaaaccaaaatcttaacaaaacgaaggatgcgaaggcgaaacaggccaaagtcgacctgttttctcattcaatcaatggttgcagtcgtgaaggagctggactcagttgcatctccaccccaagagctcgccgttagtgggcaagttggatagggtgaatgcattttcatctccgcaggcgaaagtcacgacgagggcctgttcttgtttgtttgtttgaattggttggttggtgcatcctgctggatttcaacgacggcaatcatcacgcagtgagacggagcagcagcgcatactattttctgagcaagttcatcgtttatttttcgctgtatctcgtagcgccgaggaagcaaacaagcaaacgcccaccaactgaccaaccaatgagagagccgtcctttaatttccacccctcttctgtcttccgaagattcggcggggtttcggatgggggagaaattgtggttggattggtcgggtgttttcgttttctttgattgatgcaacaatatctgctagccagcgtacaaagaatagctgcagttcaaatgaatgcatcttaattattccacaccgcggtgcctcttctttgccgtggcacaccttccgtttattacctccactcaagattttctcccc

SEQ ID NO: 4 - protein, 2360 aa, Eimeria maxima
MLHRNPRWALCAALAALYGGTGIASAEVNNELSKCESGWTPWTTCNPQTGLRERHNAQCETWVEVEECQKLTGCGNWTPWSPGDMSCVVGQFQTRNREGCPEVQEVRACRPVLLECNDQWTPWTMCDTNRVQERYNSKCGPVEVRECNMDDAEIEKCGEFVEWDPPMNGDCVRGGTHTRYRQNCPDRKEVRVCGAFDCSSCSVNATCDPIGASCECKPGFRGNGKTCEAFNPCEDTPAPCDSNAICTPDGNDAKCQCKAGWDADSGAGSSKKPCVEVDECASNTHQCPAHSTCINTKGSYKCDCNQGYVKGEDGQCHDVDECTNGEHTCPAHSTCLNTAGSYECRCDTGYSGNATADSPCKNIDECANPNACSANAICTDTDGSFTCSCPEGYSGQGTHDSPCSKIDFCADPSLNTCGAHSTCVNTLTSFKCICDAGYEGAGTRESPCVDVNECSNEKPTNNCNRNANCTNTEGSYTCECKPGFSGDGMGPNGCTDIDECAAEQSPCDPHASCSNTEGSYVCTCNTGYEPASTDGHACKDIDECATGAAGCHVSAQCLNTDGSYECKCLEGFVGDGKTCNDVDECAAATSPCGDNTHCQNTIGSYECECKAGYGNMQDNACSDIDECKDANTKIPDNCLCVNNDGSYSLEAKAGYELVNGECIKIDFCARGACNSLASCKENEEGTAAICTCLPGYSGDGTAEGHCNDIDECAGQNDCAPAEQGGICENTVGSYTCKCKEGYRQDGNSCTEIDECAEGTHNCHPSATCSNTPGSFTCQCNSGFTGSGVECEDIDECSTEADDCGANTICSNTIGAFECNCREGYERADAKTCVDIDECATGTHTCSNHATCTNTDGSFTCQCNPGFEGDGHKCEDIDFCGAGQHDCNVHAECSESEDNTTFKCTCITGYAGDGHGEAGCQDIDECAEENICGSNAVCTNTAGSYQCACREGFVASAEQQQQGTPALVCVDVDECSDASKNTCAKPADGGICTNTEGSYECACKPGYQGDGHSCADINECTAQGTCGEHTTCKNTPGSFQCDCVEGFERADERTCRDINECETGAVVLPPNSTCVNTEGSYDFDCVAGYRRTDGACVKIDFCKEKGCNANATCRENDAGTEAICTCKEGYEGSGEGEDGCQNINECERGEPCKDFGEGGVCVDTPGSFTCECAAGFIQRRSVCQDVDECLDGKLNTCAATGGVCSNTVGSFTCSCASGFEGDGHTCNDVDECATAQHTCDPNATCVNTEGSFECRCNAGFEGDGHTCADIDECADPAKNTCDTHKGVCQNTTGSYTCGCKTGFSLAADGSTCENVDECAAGTANCNERSFCKDTEGSYQCECKNGYKAAGEDCVDVDECEAGVHGCSEHAICTNTDGSYSCECMEGYQGDGKACEKTVGVCDSAPCGAHATCEPAGDNYTCTCHPGYEMREGACVDIDECTAGSLNCDPHAICTNTDGSFTCVCGSGYTGLGTSCEDIDECAGNAAGCDIHAVCTNTPGSFKCECKSGFEGDGTQCTEKVLLPGQIHCEAWTAWTECTDGAKTSTRSCLALPLKKEMRACPAADFSQCGEFTEWTACPGTNNNLSHRRTERFGEPGCEDAEEVRECPDEETEQKCGAWGEWTACGDPSPGLRTRARENCPDVVEFERCTMPSEPEAGEVTEPHTEGGAGVGGEVTEPDTEEGAGVGGEVQPGTEEGAGVGGEVQPGTEEGAGVGGEVQPGTEEGAGVGGEVQPGTEEGAGIGGEVTEPDTEGGAGVSGEPTEEEGTESTGPCKEFGPWTACKEDENGVGIQRRMCAGREDIIESRICTVTDDCGEWTPWSTCTNGSQARNKRFCTNVREVRLCGADIPVTDGCTWSEWTSCSLVNEEGGYFRTRTSSDCNMNEVQACSPSSSTTADSETEGTCSAWNPWTECSNGHQTRKCATMEAEESRTCGETPENCGEFGPFEPANCTAGQMVTRTRTCGETEQKETKLCDVSSTEEGKQCGQWGPWSECNIHLGSEDNVRVREDTACGVTEYEECSKPANNAFVCTPWSECSDKKERRTCTIRKNGLVQTRQEFRTCSVDIATTCGDFGAWSECNAEGLHQRSLEKCPDVIEVATCGSEDCPPFGEWTECGVPEEGMRSRQRIDCVESAACQCTEVESCFDTELHPIPAPGTETGEGEGETETGEGETGEAGGEEGEQTGEGEVQPPEEELPGESVTEPEEKPEEELPEEEVTEPEEKPEEGVTQPEETPEQPVEGTEEEGKQESEAAPETPAVQPKPEEGHERPEPEEEEEKKEEGGGFPTAAVAGGVGGVLLIAAVGGGVAAFTSGGGGAGAQEAEQVEFEGEDTGAATAETPEADTVIDITDEDDYWADSGDIQ

SEQ ID NO: 5 - DNA, 19056 bp, Fowl Adenovirus 8
agacctcgtcttcgccacggtcaaggagaagatcggctggcggcggttcgtggaagctatccaacggtacgtggctgacgcctacggtgctttcctgacactcaatgcggaaaccgcacccgtcgggggtgacgaagataacgccgtcagtgtgctcattgacactttaggcgaagaaagggctattttagcagcttatcgggtggcggaaaagctattagacgataagccgctgccaaacgacggcgagaacaatgggtccgaaaatccccgggacgccgcgcatacttttgcggagagtcccgaatcggacgaggacgtacaaaaggcgtccgccgagagctctcccgacacaccagctcgagactttaccgcggaagccgtaaccgtgtacatcgactcggacggcggttgcgaggacagcagcgaagaagatcaggaggaagaggaggaggacgatgaagaagaagacgaagaagaagaagacgaagaggaggaggaggaagaggaggaggaggaggacgacggaacacccgagtctaccccttctaccgtcatcgaagcggcgaatctctcgccggtcggcaccgacgaaaagcacggggaacccgacggcgagcccgatgatggtgacaatgacgacggagaggacgagggaagaaattctgacgaggatagcggatactattggggggatgacacccccctggaattggttcgcgatcgcgggacaggcgatcacggtgagccagatagcaccgctccttccgacggtcctggggaagctccgtcggccgacggggtagacgaggagcaggagcaagacgaacaagagggagagaccgccgtccccgccgccaccgctcagcccgctttcgacaaatgcctccaacggcaagccatgatgctcaccggcgctttgaaagacgccttacccgagcaggaacgcgacgtgcccctctgcgtcgatagcgtgcaataccagctcgagcgctacatctttaaccccgatatgcgtgtccctccggaataccgcgaagtgcgctttaacttctatccgcccttcatgcgccccaaagcgatcgcgaactaccacattttcgccgtcacggcgcccattccggcaagttgcaaagccaaccgcagcgggagccagctcttagaagcttgtcgcgacatgaaagtgttcaagcgcttacctcgttggcgcctcaacgtccaatccgacgacgggctcggggacgaagtggtacctgtaacagagctgacagatgccaaattagtccctctcaaggacgacatctcgcggttgcagtgggctaaaatgcgcggtgaacacatccgcttttttagctacccttccctgcacatgcctcccaagatctcacgtatgctcatggagtgtctgctccaacctttcgcaaacgaaaacgacaaggcggaacaggtcgccccctgcgtgagcgaagaggaaatgcgttttattgtagatccggagcagagaatgagaggcgaggaactctacaaggccatgctcaaaaggagggccgtcgttaccatggccgtgcggtacaccgctttgctcgagctcatggaacgcgtcttccgagagccttcctccgtcaaaaaagcccaagaagtgctccatcacacccttcatcacggcttcgtggcccaagtgcgcgaaacggccaaagtgaacctgagcaactacgccacctaccacggcgtcacctacaacgacccgctcaacaactgcacgtcagccaagcttttcgaaggcagggacaaggaggattacgtgctcgacaccgtctaccttttcttggtcctcaattggcaaaccgcgatgggtatgtggcagcaagccatcgatgataccaccctggacatctacgcgaaagcctttacgcgccagcgacgcgccatttacggcctcggaagcgtcaccgaggtcagcaaggccatcgtcgacatcctgatggacggggacaggctcacggaggaaatgcggaaagccctccccaacttcgtgacgcagagccagatctccgattttcggcactttgtcaccgaaaggtcgaacgtcccctccatggccgccccgttctacccctccgatttcgtcccgttggctttccggcaaagcgcccctctgctctgggaccaggtctacctcctccagatcgcctttttcctcaccaaccacggaggatacctgtgggaaccgcccgagagcgaagcggaggtgccgcagcaccgcacttactgcccctgcaatctctgcagcccgcaccgcatgccggcggataacgtcgctctgcacaacgaagtgctcgccatcggcactttcgagattcgcagcgccgaaggcaaatctttcaggctcacgcccgaactctgggccaacgcctatctcgataaattcgtgcccgaggacttccatcctttcaccgtgttccactttcccgaaaaccgctcttccttcaccaaaaatcacaccggttgcgtcacggaaagtccggaaatcctctctctgattcgtcagatccaggcctccagggaggagtttctcctccccgagcaaggggctctacaaagacccgcagaccggcgaaacgctcaccacttcggtcggggcagagaaccgtcctggagcctccggcggagcgcctctaccgcccgctgccgccagtacctgcggaggagctcgagcgccgccgaaacctcctagggctctacggtctgcctgccctgctgcagacccggactcccagagcgactacggggaagctgctctcgcgtccaactacggccgatatggctcagaggatgctggacgagaaaatcagagttaccgaagaccctccggaacccgagaacgccgttcccttccctacggacgcccggttcgtgggggttcgcccgtgcggaggacctgaagtgagcgaatcagacggagaaacgttagaagccggacaccgagagatctgagtaccatctcggagaggaggaggacctcgaagagatggagaaagagaatatcccaccgcggccctcctcgctgcctctggacgggacgcggaaccgcaagcgccgctccgcatcctcgcccgggaaggagctgaagaggcctccgatccgaaagagagccaaatccgataaagacgcggagaccgcgcccgcgtccaaaaagcgccgtcctcgaggtaactatagaagctgggtcaggcaccgcgtggcgatctgccaagcgctccgcgacgccgttttcgaccgaaggctggcggccgaaatcctaaagagagcgcgcggtatcttcgtaccgcccaccgtattgggctactacgcccgcaaactcctagaacttcccgacgaagatcactgatcgtcggctttttctttctcctttccttcttagcggctcccgctaccgcctctgacacgctccctccgcctctcccgccgaaaaaacgccccaaaaatacgccgcggaccgactcgtccttcgaattggtccctcccgaggtcgcagacttgaaagccaacatcctcgacgtgctcgtcgaaatcgaaaatatcgccaaaaacgacccttcacggcgcgtttccatccgcaaccgcacccgggaaagcatcactcggcagttacactacgtcaaggacgagcaaaaactcaccaagcttaaggcagatgcggaaaaaatcctgcacctgtggaaatccctttcctaatcccgcttcttttatagcgctacagaccgcgtgactgagcccgcggcaacatcatgaacctcctcgaagccactcctaccgagtacgtgtggaaatacaaccccctctccgggattcccgccggcgcgcaacagaattacggagcgaccataaactgggtggtgcccggaggtaacagtttcgcttacgcggcggacgagataagacggcacaccctaagccctgccgccacccgcgcgatcaccgaacgtttcgaagctgagtcagaccagcaacccttcgccaacgcccgggaaaccgcctacatcaccgccaacgtgctggactctggctttccaaagtccgccgtgtaccccgtggacccttccggagttcaacgggttcagctctcgggcggcgccgagggccggatgcaactcgcgggtggcctcaccgaaggtcgagtgcaactttcgggaggtgtcctaggacacgtcgtgcctcctggggggagaagacgcgccggcgggcgtccgccgcgatggtgtgggaccgctctcgcgggaaacgggcttcccgaggacgccgaagtggtttcggatacctacaagtacttcctccgcacccagggacccagccaagtcgtgcaagaacccggcgtgtactcgcggaggcagttcatgaccaccttcctgccggccgtggtgccccgacctttcagcagtcccaatccgcgcgactttcccgcgcagtacagcgccatctacaaaggcaccaacgcgtacgaggacgtattttgggactggtgaagtccctcttcgcggcttacccgttgctgacggtgctctgtttcgcaataaagttcttccaattcagcctcgctgaacggttcccgcctcgttattgtcacgcgttcgcctccgtcgctcaccacgcgcgcgcgaaaccgtcttttgatccaaaagacgtaaccggggtttaggggttgcgcaaacctcacgatcgcctggtcgttgactttcaaccaatattttttaggagcctgcgactccgtctccgacatggcgacctcgactcctcacgccttctcctttggccaaatcggctcccgaaaacgccctgcgggtggcgatggcgagcgagacgcctccaaagtgccgaaaatgcagacccccgctccgagcgcgaccgccaacggaaatgacgagctggacctggtctaccccttttggctccaaaacggctctaccggaggaggcggcggcggcggttccggtggaaacccgtccctcaacccgccgtttttggaccccaacggacccctggccgtccaaaacagcctcctgaaggtcaataccgcagcccccatcaccgtcaccaataaggccctgacactcgcctatgaaccggagagtctcgagctcactaaccaacagcaactggcggtcaaaatcgaccccgaaggacctctgaaagccacgaccgagggaatacagctgtcggtcgaccctacgacgttggaggttgatgacgtcgactgggagttaaccgtgaaactcgaccccgatggccccctggattcctcagccgcaggaatcacggtccgagtcgatgagaccttgctcatcgaagatgctggatccggacagggcaaagaactcggagtcaatctcaaccccacgggaccgattacggccgacgaacagggcctggacttagaaatagacaaccagacactcaaggtcaacagtgtcaccggcgggggcgtcctagctgtacaactcaaatcccaaggtggacttaccgtacagactgacggtatccaagtgaacactcaaaacagcatcaccgttactaacggagctctggacgtgaaagtagccgccaacggacctttggaatcaaccgacaccgggctcacacttaattatgaccccggagacttcacagttaatgcgggcacgttgagcattattagggacccggctctcgtagccaatgcgtacctcacatccggcgcctccacccttcagcaatttacagctaagagtgaaaattccagtcaattttctttcccatgcgcatactatctgcaacagtggctttccgatgggttgatttttagctccctctatctgaagctcgacaggcacagttcacgaacataccaacgggtgaaaattatcagaacgccaagtactttaccttctgggttggagcgggcacttcatttaatctttctacccttacccaacccactattacacccaacaccacacaatggaatgcattcgcacctgctcttgattactcaggtgctcctcccttcatctacgacgcgtcttccgtagttacaatttattttgaacccaccagtggtcgactggaaagctatctccccgtccttaccgataactggagccaaacaacctacaaccccggcaccatcaccctgtgtgtaagaacggtaagggttcaattgagatcgcaagggaccttcagcactctagtctgttacaacttccgctgtcagaacgcgggcatttttaacagcaacgctacaacgggaaccatgaccctgggtcctatcttctgtagttatcctgccttgagcaccgccaacgctccttaattcaataaaaaatgatccacacaatatgaaggtctactgtgattttttattaaagcagccatactaattctcctggatacccatcagtctgtcccactctccgcgttgccagtagtacaggcagttcacggcgtccacgtaccacgattcgctcaccagaaagacccgctgctcgggaaaatccaccatcattctgcggatgtagtgacaagggagcgcccccatctggccgagcgtggccaccgcttccacgaacacaccggtgttgtgcggagggacatagatgatcatgcccagaactcctccgcgggcgcggcgcagaagacgggataaaattcggtaaacatgacagaggcccacgccgctacgaagtacaccccttccagactagggtcgccttccagcctgctccaaaggtacacggagagaccactgctgtcgggttgactgcacacggccatcggcacgcgtctcatctcgaatccgtggcagtgacaccgctccggaatcatctcgaattcttccaaacatagaaactggtgcgcgtggacggccggcacgaaacgcaaatctccttcccctgctcccaccgcgggttgatgttcccctatgtcttcgccccataacctctgagcggccatgatctacacctgggattcttcgcgctctatctcagtatacacgtgttccacagagccgccgtagacctcttcctcgtcgctacctgccggtggcgctctcagcaacgacagttccagtggtgtcggcggcggtggaacagaaggaggcggtgaacgggtatccgagcgggagtcgtaaaacggattgctgctcacagtccaattgggggaacgagcgggaaactgagacagggaacgcagccaggagaaccgacgtgatggctcgcggttattaggcaatggtgggggtaaagcagaacaccggcgacggatactccaacggtgtcctcggatgctcttgaggacctcccgcaatgattctccgccactgcgcgatcagcaatacaaacgcgatcgtggctagaagagtgcaacagccaaacatcataaacacgtagggaacggcatgtaaatattgactgaggaagagataactggcggccaccgcaccgcagtgaatgactcccacggtcacagccagaagcagcagaaggcatgcctctctgcggcgccggcggatccgcacctatcaatagaaaaaaggggactttctatcaccctccacgcgtgcccggcgcttggacatgcaattccgcaaataggacaactgagctatagtggctaggggcaagggctgtctaagagggcatccggggcaagaagcttcggggtgatggtcgcagtacgtgccgtgaacatgcagtacccattcttctacatccacaaacgtggcggtacgggaagcggaatgtagcataacccccgcgacaccatgctccagcaagcggggtcggccatctctttcagcatggatcgggtcatcagaggctgggtcacgggactgcccttcccgcaagttttaagaatgacggtcgctaccacattggtgcgacacgcacccagatagagaacgggatatttcaaaaaaggcaggtgctcaggcacgaccgtctgggaaacctcataacataatgattgaagagccaaccgaaaggcaacaccggtctctagaacgagtcccgtatctacaaacagaaagtcggttttgtttttgcgaaccatccattcccgatgtttctctatcagaggttcccgcgctacgaacagacgcctcgaaaccgctcgcaggatctcctccttttccgcgggtgataaagacaaccgagactgcagtctcagtatgacgttcaacagaacgcacggtcccgtcttgagtctgagatacttcgaacacctgcagctcactaccgtatacagggactcgtgccacgagtaatgctggggtttatcgaagagactaatggaggctacggaacggctcgtgtgatactccatcatgcgttccgctgcttcttgggacggaccctgtctgaccaggatgctgaaccatatggctccgcattcgttttgatagccgcaaccgcgggtaacggcaaccacctcctacacgaaagaaaggggcgccttaagttactcaaggaaaccgcccgggaaaaatcggggcaatgaaaagctatcactcaccgaatcagaacacagaggcatgatgcgtaactaagacagctcttttattgatcaggtaccgtcacctgtaaagatacacacattaaacgatacggtaagagtcaccgcggtaacaccgacatcggtagtggcagaatatatagagcacgactgctgtcgtgaacagagcagctacgacaccacccgtaacaatcgcgaggcgcgcggcatcgtcttcccaaaattcacggatcagagagtagaactctcctccgagctgaggagacgtagggaaggatcccgtagacccattgctacttttttccgtcggatagcggtagagaccaacacagctaccatagccaaaaacacaagccccacaaccgttaaaaaaagagttccggtagatgcacccgaggccgctactcgagagccctcgtctatggctcgcaaggcgacggcttgaaccggttcggtttcgtttagctggacggtcactacctcctcttctgacgcggtttccgaagtgctccaaaaatcgcttgaactcggtgtagctgctgagaaattccaggtcgaaacggtcgatgcggagtctgtcgatgtagagttcaaagacgcagtaggttctggcgggaactccacagaaacgggttccagtgggcttaccgttaccttcaattttataacttcaaattcaagaaagaattccagttcgaatacgtcggcattaaagaaggtgacggtaaattctttcttcattctgtccaattggaagacacactgcgcagctgtctgacacacgtcccagaaactgtacagtttatgggtccccgaagaatccgtaagttggatactcgtcagccagaaaggagacttgctaagatctaccttgagtccatgtcccactttcacttctacatcagccagctggatcggaatcatagctatcgaggcatctctatccgccaggcaaccagactgaggaggaacactgacttgagatccgccgttcggatttaacatcgtgcgataactcattagggggaatcgctccttggttaccatgcagtagtgcgcaggccgaccgaacggatctcttgtagggttacagtgcatggtacgcacacaccctccagtcattactttacggtcctttatcgagggagcgcttgacgaatcctgatagaatccctgtggtgtgatcacgtacaccaggtagatacttgcagaaggattccagtgacggatccacaacacctcttctgcaagctcggtgtaacccaccgcggtaagtacatcatacctcccataccgaagtgtttgctcactgtaacctccgatgaacattcgactcccactagaagctactactatgatcatgatcggagttacaaagtcctgcttgagtacgggtgagtacgtaatctcagtcatgtagctaatgctaatatgattgaagtagctagcgtatctgtaatctcgactcatagtatacccattccaagtgctccggacaggacccactttgaaaccctcattaagcaagtgaaacatgggtaacccagttctcacatccaaagtctttagaaacttcggtactatcaatctatgaaaacagccgcttttactccacgtatccctccatgagaattcttcgcagaccgtctgtccccatacactttctgcacacactcttcccgaccatcccccatgaatcccacaacctctaccgcccttatcctccaacatgatatcctcagaagccgtataccctcctgtaatccacgacccttccgttttcaggactgccacgtaatccgcatcatttttatctaagcctccggtaacaaacgtgggctctaacccgactattctggtacaccgtctatccccagaaatatatgtccactgtctgcagatagtagagcaagcgtgagcgccggcagaatgtcctatacagtgtaaccttttcgaatgtggtatgtccgacagaaacttcccgaaatcaacatacagaccgtcatagtcgtagtacaggttccagtccaccaggagcagcgcgactgcaggcgtcatattctgatggacacgcaacacctgtctaaaactttgatagcccatgtaataccccggtataagcacaatgatatcggtcttgccggctaaagcactgtgcaatcgatggtagacaccgtactggtacgtcacgtcgtgaaatttcggcacacccccgtgagctccataccacgtaatcttcgatggcggtcggctcaatcttccaacgcctctcggagcatcaagttttagagaatccggtctcgccacgcatgaccgattgcccgaatcgggacatgcaactgccgatttcgcacacgaaagcacggaggctccgaagagcaccccgaccaactgaaacagaacgcatgatttagacccactcccgacagaggattaacgcgacccaatcaagggatatcaaaaaagaatccttaccgcggagagattcatagaccgaagagttgagagcgctcctgtttctctgaagatctctcaccccgactgtgacagatccacaaagcagcactcaatttatactgtcaaatggttaatgtttaatgctagaaaagcgctgacacccagtaaatatttacttagtttgcagttccactgtttccttattgccatgactggacaaaaaccacagataagatgttccattcaagggaacccgatgttccctcgataacttcccggtacaaagtccaaaaatagaactaggtgctttataaatactaagagtcgactccttggtgtttcagaagaacacagacgatctacaaacaggatgaacctcggaagactcaacaccgccggtaagaacatcttaatttttactttgtatgattttcaattctgaaaaacacgtttcctggttcgtgcacgtacgcggaaacgaagttcgaaaaatcagagttggaattttccagctatggttaactattaactatatgacgtcacttagttaattattaacgatataaagtaaatgattaactcgggctagttaatgattaactatacctggttaatgattaactgacttagttaatgattaactagaagttaatgattaactagaagttaatgattaactagaagttaatgattaatctattacgtcactcgttatatattaactagtgacgtcactcgttatacattaacccattacgtcactcgttatacattaactagtgacgtcatgagtaaatcattaaccttcatgcatatgcatgaggagctactgaatatgcatgagagcctcatacatatgcatggaacttatgcatattcacgacactcatgcatatgcatgcattggttaaagagtaaccctatgactcagtgtgtatgtttacgttgcctagcaacgttaatgatttacctgctgacgtggcagctccgcctccaggtaaatcatttacctgaactttgttctttatgtttattcaccatggcaacgctaccatatatggacatccgactccgcctcccccgttatacattaacgatggcgtgataggcggagctctcccccattggctctcaatgacgtagttcaggttaaccataagccagaaccgcctatataggtagagcaggtagacccggaacaccattcccatccggacctccatagagtgcggacctctacgggctctccataccggtaaatattttattccatttaatccaatcgaataaatcaataatcaactcaatgctgtgattctgcctcaaattcaatggtgattttctttaataaaaagcccaccccccttggcacccccctgtacacccccctgtacaggcgaccaccccctatggacacccccctgtacaggcgaccaccccctatggacacccccctgtacaggcgaccaccccctatggacacccccctgtacacccccctgtacaggcgaccaccccctatggacacccccctgtacaggcgaccaccccctatggacacccccctgtacacccccctgtacattttctcccataggctacaatggaatactgccccctagtgtctcctgctgtatgggacccctatgatgtgggcgccattacctttgccactatggagctccttcacgagggggcgccattgaaattgggagaccgcatagagagcctagccaatggggtgctttggaatccggatatccccgtccaactcttcaactgcctttccattcgctcatggggatcacatgggaagcgcgtcatgtaccgtggccacacctaccggatgtaccacgcccagttacgggtccgaagctccgcccccgttactaggaaacaggccggaagcctgctcctcagcctatcacagaagctcctctgtttcgccgcccgctttaatacccatcccctcgtgatgcaattgggggtggagtctaaccctatgggcctacctgtatataccaagagggccctccagatggcgctacagagtatgcgggtgcgcattgcccctgacggccagaaggtggcgccaccggagataggcaagacctgtacggtgaagcccctcaagaccccggagaccctccagcagggggtcttcagtaccaccgatttaaaaaagacacttccagattgggcttttcgccgactttttaaccaaaccccctatatttgtggatggaagattggcaccgcgccagaaggggcggagagttggatcgttacgctccacccccagccttcgactccgccccccacagggaccaagactccgcccactctgcaggaccttgcccggctgggcgtggtcgagcaatgcctcaagatgaggaggcgtggcctggaccgcaggcaccacccctatgctcaataaaccaatcagattccagtacttggctcctcctatttgtgggcgggactttgcacgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggtgcgggcccgagtcacggatggagtagtttcccttgcggccagcagagggcatacctttattctcagctcgcaagtctcaatagatacacacctcatcggtgtacagcgtgtccgcgtagcgcagccccgtgcacctcacccaaccacctatatcgcgaacggctccggtactcactatgtatttcccgacgcgatagttcggatcattgcaccacttattcaagtacattctaaaccattgcccttcggggacttggcgctgataaaaacattccctgaagtaccgtttcaccgcgcgagaacacttatacaagtatctgtcccgcaggttgaacatggttaagcacagaagcaaggtcatgtggcaggaacaagaaccgccaggctgcaaccccacgcagtatcccatcggatgaccgatctccgagttcgcctcctcgtggaacgggtaccatagctgcttcacgtcttgaaccagcttccagaacactgcatcgtgcatacaccacggcggcacggcaactccgaagatcacgtacagtggcatgttccggatacatctacgacacaggtactgtgacaccttgcgcgtacgcgaaaagggaacccgaccctcccccgtaacgctccacttacggacagccggcgatgcgcactgcgaacgaaaaataaatctgcgccgttgtgcgctccaggcggaaacaggggaatatataagccaactcttatctttattgttgccacgcccgacactatccagatttcgagacctgctgacaccaccggaacacgcgacctcgccctctctttatcatccatacgcccaggtgactcagtcaaatcccttatataaagaccgtttttacctgaccgcttccacgtacacaaggcggcacatgaaagcaccatgctgcgccccgtatccgccatcgcgttcctctcgtgcctatgtctcacgcggaccgcgcaacaggtcggtaagtcctttaatcttacgacccactccaacctcactgtgcacccgcagaccaaaggaacctcaaagcaagagtggcggctagggcgcgataccaagattgcgatgtgggagaaaggctacgggtacagctacccgtcgggaccctttaaaggccgcgtagaaatgaacgagaccagtgtcaccttttttgacctccgtcccaacgattctgccatactgacttacttctccgaagatagctccggcacggagagtgaatatccgtacgccatcagcgtaagaggtgagcccttccctacctttgttccattccgcccatcgagcacctcagccgacaccacacattttcagatcccctccgccctcccattctacggctgatgactaacaattcccgtccgcggaccgagagccgcatgagcttgcagtgcatcgcgctcgataacgatagttccattacgtacgcctggtacactgacaccttagagagcggggacaacatccgagaagtaaccgtccgaacggattctgaggtagcagttacctgtcggatatcggatggacattccaccaattccgcgactctcgtcgtgccgctaaaccgaggtcagtaatatcccctccctcaccgcaacagatccgcacagtcaggatcccaggctttcacgatcctttccccactcctttagaacctgccgctccctacggcgcggatatgactacggtgttcctggccatcttagcccttattcttctaaccgtcatcggcggctacgccctcagaaagctgtgtatgcgaaacgagcgcgtttttatttgtaacccgtacagagaatgttttggcggtcatctctaggacaaataaacttctacttgaaatgagtttatttttccccctgcctgtttgtgatgggaaatgatcggtgctgcttatggaccgagatagatggaagggacgggggcattcaaatttctaggtccagggacataaaaaagagatcaaatttacatctccggtaaagatcacctctataaccccgctgtgaatcccagcactcccttccgatacgcaaactgactagcagttcctgtgtatagacaaacggaatcctggtgtacagacaaacggaatcctgagttcccaacgcattcatttatttgaatatttacacatttacacactgtacacggtcattcgatttcattgccaacagaaagactaatcgatgtcccctttcagtatgtggactgtgacagcagggtcttcgctcacttcactgtccgtgtcatcctctgtacgcccacacagcatcgcccgatagtgaaagctgacactcagcatggagaaccaaacagcagggagcaatgtgagaggcagccaacacagggaaacttttttcttctcccttcagaccccctcccgtcgagacattgtgatggactactggtacttcgccctgatgttcccaggtaggaacgaccgtaagggtgaacctctgaacgctgttctgcatcaggtcccgtgtcacttgatacatgccggaatccgcgccacttaagttcttgatagtcacgcagttctcggatctgttatacgacagcctcccttgatacgccccgtagaccatcggctccttcagcgcctggtagtcctgcagcacgaacttgcgcacggcgcccgcatccaccgcggtcacttcccattctctgatctgaaaactctcggtagaaccgcacagagtcacagcgtcccgttcgttagccacgacccgggatacgttctccattttcaccgtaccgttctcgggaagccccgacacggtgaaatatacagtctcgggcttggaacccaccgcgaatgattgtgatagccagcacccgttattagctttgattgcttcgacatgcattgaattacaggatgaatttagccgcgccctcgtcatatagcccagatccagcccctctttgatcgaaggaatcacgaaagccactttctgccatctgttacagaccttccccggagcgtggtaccatagcagcagcgtgaaatcagcgcatctgcccgtagtcagagtcacctccttacggcccctcacggcggcaccggagacggtggcagacaagcagaggacggcggcacacagcaggagcgagccatgactgcggagtccgagccgagcggtgtgctgctcgatcctccgctacctttttatgcaccacccacctttattgtcggtcacacattaattcgccccgtcagcaaacacgtgagtaacgtatgccgttgttctgatcggtcagcaccgcgcccgcgacgtttgaacgaagacgtacggtgacttccgcatagggagctataaggaagtcagttaggaaaaatcgatcctcgacacaccccacggaaacgctgacctaggcgaaacctatcaggtaaaacattcactatacgcaccacgcgttgaataaacagttaacccatacggggtatatattctaccccgactgcagtcagcgatcaggacgcgtccacagagagatccgtgcgcgaccgcctgtccgggctcctctagaatcaagaattccataaccatgcaggtaagaactcctttctcttcctcctatttgatccgagcctttttttacgctactcaaccgcaacccgtttcttccaccacagttactgctccttctatgcctttgccctctgatgggcagcggaacactagcacccctcgtctcggtagacaactcctactccgtgttcggatccggcaaacccccactttctacctccgaatccgccaccaccgcctactccgaaaccgcggttcccgaaaactcttacccccatccgaagccaccacgccctgcgacgacttgctggaagaggactgctggttcgcggagagcagcgcggactacgcacccataccctggaacaccaaagagaatacgtccgtggttatcccggcacaggtagccgtctcgccatcgcagtccactactccccctgcggtcatgctcggcatcgcacagaaagccgtaaaccgcggagcctccagcaaggatcacacgtcccatatcgccacgggcgttaccgtagccggaatagtcatactcattgccctcgtcatcatagccttccgtacaaaggttaaggaaccgcgcccaacccgctccatctacctgggcgtgcctccccctgacgttagaccttaccgtataatagagcaataaagatttggccgccacatcgcacaagaatctttccgtgtcctgtgtctgtctcggcgccgtccgcgggaaaaggttaacgcggaatctatttccctgcggatttccgtatccgtcagttcctgggcgtcgccgaaaatgctcacggaagacacgcccatgcgggcgtggctaaccgatgattcgaaaaacgattcgcgagcgccctctgccggcggcggcgggaatagggggtgtggggggagtgtattttaagtagatatatatagatgatg

SEQ ID NO: 6 - protein, 27 aa, Gallus gallus
MTCQTYNLFVLSVIMIYYGHTASSLNL

SEQ ID NO: 7 - DNA, 78 bp, Gallus gallus
acttgccagacttacaacttgtttgttctgtctgtcatcatgatttattatggacatactgcaagtagtctaaatctt

SEQ ID NO: 8 - protein, 23 aa, Sus scrofa
MSYTTYFLAFQLCVTLCFSGSYC

SEQ ID NO: 9 - DNA, 69 bp, Sus scrofa
atgagttatacaacttatttcttagcttttcagctttgcgtgactttgtgtttttctggctcttactgc

SEQ ID NO: 10 - protein, 20 aa, Influenza A virus
MKVKLLILLCTFTATYADTI

SEQ ID NO: 11 - DNA, 60 bp, Influenza A virus
atgaaagtaaaactactgatcctgttatgtacatttacagctacatatgcagacacaata

SEQ ID NO: 12 - protein, 335 aa, Eimeria tenella
MTRLSLCALAVALAVGQSLAVPTTVENTVHPYSEMGTYQEGEAPGAPDESSTTTTTPSPSPEAPDQWLENFVRAVQRQLQLQESMMRQLVKEIQEYLSRAFNWDENQSAAYNRVNEMMDMITNRMTTALDGANELMATSETMDPETLRRATRKYMKEVRVQDVVVDSLWASLRGGVQTSAWMSGVTAVEKEETTPMAGRAAEEFMHRMYHNLRAAGMAEEDITRFMPKTEYTTPREQTRNMGRKGRYGYGYSYGYPLYSYGYSYPSYSYSYPYYSYPSYSYPLYSYSYRYPSYSYSYPLYSYSSYSYPYYSYSYPYYSSSWYWRRLRTASCPDCP

SEQ ID NO: 13 - DNA, 966 bp, Eimeria maxima
accactcctgttgagaaccaggttcacccttacagcgagatgagtacctaccaggaggggagtgccccgggggctccggaggacaccaccaccaccactacgtcgtcccctgtttccgatggagccgagcagtggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccatggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaact

SEQ ID NO: 14 - DNA, 1431 bp, Eimeria maxima
atgacccgcctcggcctcgctgctgtcgcgctggctctcgccgtgggcccttccatggcagtgcccagcaccactcctgttgagaaccaggttcacccttacagcgagatgagtacctaccaggaggggagtgccccgggggctccggaggacaccaccaccaccactacgtcgtcccctgtttccgatggagccgagcagtggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccatggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaactccccccccaatgatgaacatgatgaacaccccaccccccatggcaaacatgatgaccagcatgatgatgaacactcccatggttcctcctccccgcaccctcggaactgaagccatgagcctcggcttggcccccatcggtatcaccggcgcccccatgacaggtttcggtgttcctcctgagttcggtccctttggagccgaaggtatcggcctccccaccgatgccctcggcagcacccccgaaatgacaccattcgacccaactaccccctacagaactctcgcccccatggacctcccccccatcccccctcctgtcttccctgaaacccctatgaggccacctactcccttcggcttcggacctgcacctgttcctcccatgcccttctaa

SEQ ID NO: 15 - DNA, 653 bp, Eimeria maxima
acaccgaattgcaccccattccagcccccggtacggaaacaggcgaaggagagggagagaccgagacaggcgaaggcgaaactggtgaagcaggtggcgaggaaggcgagcaaacaggagaaggcgaagtgcagcccccagaagaagagcttcctggggagagtgtaactgagcctgaggagaagcctgaggaggagctacctgaggaggaggttactgagcctgaggagaagcctgaggagggtgtgactcagcctgaggagacacctgagcagcctgttgagggtaccgaagaagagggcaagcaggagtctgaggctgcccccgaaactcctgccgtccagccaaaaccagaggagggtcacgaacgcccagaacccgaagaggaggaggagaagaaggaagaaggcggcggcttcccaacagctgcagtggcaggaggtgttggtggtgtgttgctcatagctgctgtaggtggtggtgttgcagccttcactagcggcggaggtggcgctggcgcacaggaggcagaacaggtcgagttcgaaggagaagataccggagcagcaactgccgagacacctgaagccgatacagttatcgacatcacagacgaagacgactactgggccgacagcggcgacattcagtaa

SEQ ID NO: 16 - DNA, 7083 bp, Eimeria maxima
atgctgcatcgcaacccgcggtgggcgctttgtgcagccctcgctgcactctatggcggaacaggaatcgccagcgccgaagttaacaatgaattgagcaagtgcgaatctgggtggacaccctggactacctgcaacccgcaaactggtctgcgggagaggcacaatgcacagtgcgagacatgggtggaggttgaggaatgccagaagctgacaggatgtggcaactggactccttggtctcccggcgatatgtcgtgtgtggtgggacagtttcaaacccgcaacagggagggctgcccagaggtgcaggaagtgagggcatgcaggcctgtacttctagaatgcaacgatcaatggaccccctggacaatgtgcgacaccaaccgcgtccaggaaagatacaactcaaagtgcggacccgtcgaagtccgcgagtgcaacatggacgacgcagagatcgagaaatgcggcgagttcgtggaatgggatccccctatgaatggagactgcgtacgcgggggtacccacacgcgttaccgtcaaaactgcccagaccgcaaagaggtgcgggtgtgcggagcctttgattgcagtagctgctctgtaaacgccacttgcgatcccattggtgcatcctgcgaatgcaagcctggtttccgcggcaatgggaagacctgcgaggccttcaacccctgcgaagatacccctgcaccttgcgacagcaacgccatctgcaccccagacggcaatgacgccaaatgccagtgcaaggcaggctgggacgcagattccggagcaggcagcagcaagaagccttgcgttgaggtcgacgagtgcgcatccaacacccaccagtgcccggcacactccacatgcatcaacaccaagggctcttataagtgcgactgcaaccagggatacgtcaagggagaggacggacagtgtcatgacgtcgatgaatgcaccaacggagagcacacctgccccgctcactccacttgtttgaatacagctggcagctacgagtgccgctgcgacactgggtacagcggaaatgcaactgcagacagcccttgcaagaacattgacgaatgcgccaaccccaacgcctgctcggccaacgctatctgcacagacaccgacggctccttcacctgcagctgccccgaagggtacagcggccagggaacccatgactctccctgctccaagatcgacttctgcgcagacccctcactcaatacatgcggagcccactccacttgcgtgaacaccctcacatctttcaagtgcatctgcgatgcgggatatgaaggcgccggcactcgcgagagcccgtgcgtggacgtgaacgagtgctcgaacgagaagcccacaaacaactgcaacagaaacgcaaactgcaccaacaccgagggatcctacacttgcgaatgcaagcccggtttctctggcgacggcatgggtcccaacgggtgtaccgacatcgacgagtgcgcggcggagcagtccccctgcgaccctcacgcctcctgcagcaacactgagggctcgtatgtatgcacctgcaacaccggctacgagccagcttcaaccgacgggcatgcatgcaaagatatcgacgagtgcgccaccggtgcagctgggtgccacgtgtcagcacagtgtctgaacacggacggcagctacgagtgcaagtgtcttgagggcttcgtcggcgacggaaagacctgcaacgacgtcgatgagtgcgctgcggcgacatctccttgcggtgacaacactcactgccagaacacaattggcagctacgagtgcgagtgcaaggctggctatggcaacatgcaagacaacgcatgcagcgacattgacgagtgcaaggatgcgaacaccaagatccctgacaactgtctttgcgtgaacaatgatggcagctactcccttgaggcgaaggctggatacgaattggtgaacggcgagtgcatcaagatcgacttctgcgcccgcggcgcatgcaactcgctggcctcctgcaaggagaatgaagaaggcacagcggcgatctgcacctgcctgccaggctacagcggcgacggcactgctgaaggccactgcaacgacattgacgagtgtgcaggtcagaatgactgtgctcctgccgagcagggaggcatctgcgagaacactgtcggctcgtacacctgcaagtgcaaagaggggtacaggcaagatggaaactcatgcactgagatcgacgagtgcgctgagggaacccacaactgccacccttccgccacctgcagcaacacccccggaagcttcacctgccaatgcaacagtggattcactggcagcggtgtggagtgcgaagacattgacgagtgctcaactgaggcagatgattgtggtgcaaacaccatctgcagcaacaccattggtgctttcgagtgcaactgccgtgaaggctatgaacgcgcagacgcaaagacgtgcgtcgacatcgacgaatgcgcgacaggcacacacacttgctcgaaccacgccacctgcaccaataccgatgggtcattcacatgccagtgcaaccccggcttcgaaggtgacggccacaagtgcgaggacatcgacttctgcggtgctggacagcacgactgcaatgtgcatgccgagtgctctgagagcgaggacaacaccactttcaagtgcacctgtataacagggtacgctggagacggccatggcgaggcaggctgccaagacattgatgagtgcgcagaagaaaacatctgcggaagcaacgctgtctgcacaaacaccgcaggaagctaccaatgcgcatgccgtgagggcttcgttgcatcagctgaacagcagcagcagggaaccccagcactggtttgcgtggacgtcgacgagtgcagcgacgcttcgaagaacacatgtgccaagccagccgacggaggcatttgcacaaacactgaaggcagctacgaatgcgcttgcaagccaggctaccaaggtgacggccacagctgcgcagacatcaacgaatgcactgcacagggcacctgcggcgaacacacaacttgcaagaacacacccggatccttccagtgcgactgcgttgagggattcgagcgcgctgatgaacgcacctgccgtgacatcaacgagtgcgagacaggagcagtcgtgctgccaccgaactccacctgcgtcaacactgaaggcagctacgacttcgactgcgttgctgggtaccgccgcactgatggagcttgtgtgaagatcgacttctgcaaggagaagggatgcaacgcaaacgccacatgccgcgaaaacgatgccggcaccgaggccatctgcacttgcaaggaaggctatgaaggcagcggagaaggcgaagatggttgccagaacatcaatgagtgcgagagaggcgaaccctgcaaggacttcggcgaaggcggtgtttgcgtcgacacaccaggatcattcacttgcgagtgcgctgctggattcattcaacgccgctccgtttgccaagatgttgacgaatgtctcgacggaaagctgaacacctgcgctgccaccggaggcgtctgctccaacaccgtcggttccttcacctgctcgtgcgccagcggcttcgaaggcgatggccacacctgcaatgatgtcgacgaatgcgcaacagcacagcacacctgtgacccgaatgccacttgcgtcaacaccgaaggcagcttcgagtgccgctgcaatgccggattcgagggcgacggacacacctgcgcagacatcgacgaatgcgcagacccagccaaaaacacatgcgatacacacaagggtgtatgccaaaacaccacagggtcctacacctgcggctgcaagaccggattcagtcttgcagctgacggaagcacatgcgaaaacgtcgacgagtgcgcggcgggaactgcaaactgcaacgagcgaagcttctgtaaggacacagagggttcctaccaatgcgagtgcaagaacggctacaaggctgcaggagaggactgtgtggacgttgacgagtgcgaggctggcgtgcatggatgcagcgagcacgcaatctgcacaaatacagacggcagctactcctgcgaatgcatggagggataccagggagacggcaaggcttgcgagaagacagtcggcgtctgcgactccgctccctgcggtgcccacgccacctgcgagcctgcaggggacaactacacttgcacatgccacccaggctacgagatgcgcgaaggagcctgcgttgacatcgatgagtgcacagcaggcagcctcaactgcgaccctcatgccatttgcacaaacaccgacggctccttcacttgcgtctgtggcagcggctataccggccttggcacatcctgcgaagacatcgacgagtgcgcgggtaacgcagcaggctgcgacatccacgccgtctgcacgaacactcccggatcgttcaagtgcgagtgcaagagcggcttcgaaggcgatggcacgcaatgcacggagaaggtgttgctccccggacagattcactgcgaagcctggactgcatggacagagtgtaccgacggcgccaaaaccagcacacgcagctgccttgcactgccgcttaagaaggagatgcgcgcctgccctgcagctgacttctcccagtgcggagagttcactgaatggactgcctgccctggaaccaacaataacctgtctcataggcgcactgaaagattcggagaacccggatgcgaagatgcagaggaagtccgcgaatgcccagatgaagagaccgagcagaaatgcggcgcctggggtgagtggaccgcctgcggcgacccatcccctggcctgagaactcgcgcacgcgagaactgccccgatgtggtagagttcgagcgttgcactatgcccagtgagcctgaggctggcgaagtgactgagcctcacacagaaggaggagccggagttggtggcgaagtgactgagcctgacacggaagaaggagccggagttggtggtgaagtgcagcccggtacagaagaaggagcaggagttggtggtgaagtgcagcccggtacagaagaaggagccggagttggtggtgaagtgcagcccggtacagaagaaggagccggagttggtggtgaagtgcagcccggtacggaagaaggagccggcattggtggcgaagtgactgagcctgacaccgaaggaggagccggagttagtggcgaaccgaccgaagaagagggcaccgaaagcaccggtccatgcaaagagttcggaccctggacggcctgcaaggaggacgagaacggagtcggcatccaacgccgtatgtgcgccggcagagaagacatcatcgaatccagaatttgcactgtcacggatgactgcggagaatggaccccctggtcaacttgcactaacggcagccaggccagaaacaaacgcttctgcaccaacgttagggaagtccgtctctgcggagctgacattccagttacagacggatgcacgtggagcgagtggacttcttgcagtctagtcaatgaggagggcggctacttccgcacgcgcacatcctctgactgcaacatgaatgaagtgcaggcctgctctcccagcagcagcacaaccgcagacagcgaaacagaaggcacctgctctgcatggaacccctggacggagtgctcgaacggccaccagacacgcaagtgtgccacaatggaagcagaagaatcgcgcacttgcggagagactccagagaactgcggagaattcggccccttcgaacccgcaaactgcacggccggccaaatggtcaccaggacgcgcacctgcggagaaaccgagcagaaggaaaccaaactgtgcgacgtcagctccaccgaagaaggaaaacaatgcggtcagtggggcccatggagcgaatgcaacatccacctgggctcagaggacaatgtgcgtgttcgtgaggacaccgcttgcggcgtgacggagtacgaggagtgcagcaagccggcgaacaacgcctttgtctgcacaccttggagtgaatgctcggacaagaaggagcggagaacgtgcaccatccgcaaaaacggtcttgttcagacacgtcaagaattcagaacatgcagtgtagacatcgccacaacttgcggcgatttcggcgcatggtctgaatgcaacgctgagggcttgcatcagcgcagtctcgagaaatgccccgacgtcatcgaggtcgcaacttgcggcagtgaggattgcccgccattcggcgagtggactgaatgcggcgttccagaggagggcatgcgttctcgccaacgcattgactgcgttgaatctgcagcctgccagtgcacagaagtggagagctgcttcgacaccgaattgcaccccattccagcccccggtacggaaacaggcgaaggagagggagagaccgagacaggcgaaggcgaaactggtgaagcaggtggcgaggaaggcgagcaaacaggagaaggcgaagtgcagcccccagaagaagagcttcctggggagagtgtaactgagcctgaggagaagcctgaggaggagctacctgaggaggaggttactgagcctgaggagaagcctgaggagggtgtgactcagcctgaggagacacctgagcagcctgttgagggtaccgaagaagagggcaagcaggagtctgaggctgcccccgaaactcctgccgtccagccaaaaccagaggagggtcacgaacgcccagaacccgaagaggaggaggagaagaaggaagaaggcggcggcttcccaacagctgcagtggcaggaggtgttggtggtgtgttgctcatagctgctgtaggtggtggtgttgcagccttcactagcggcggaggtggcgctggcgcacaggaggcagaacaggtcgagttcgaaggagaagataccggagcagcaactgccgagacacctgaagccgatacagttatcgacatcacagacgaagacgactactgggccgacagcggcgacattcagtaa

SEQ ID NO: 17 - DNA, 13891 bp, Artificial Sequence
  Plasmid
cggatccctcgaggtcgacgaattcgagctcggccgacttggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttatgcatgcggccggccgcagctagcgtatctgtaatctcgactcatagtatacccattccaagtgctccggacaggacccactttgaaaccctcattaagcaagtgaaacatgggtaacccagttctcacatccaaagtctttagaaacttcggtactatcaatctatgaaaacagccgcttttactccacgtatccctccatgagaattcttcgcagaccgtctgtccccatacactttctgcacacactcttcccgaccatcccccatgaatcccacaacctctaccgcccttatcctccaacatgatatcctcagaagccgtataccctcctgtaatccacgacccttccgttttcaggactgccacgtaatccgcatcatttttatctaagcctccggtaacaaacgtgggctctaacccgactattctggtacaccgtctatccccagaaatatatgtccactgtctgcagatagtagagcaagcgtgagcgccggcagaatgtcctatacagtgtaaccttttcgaatgtggtatgtccgacagaaacttcccgaaatcaacatacagaccgtcatagtcgtagtacaggttccagtccaccaggagcagcgcgactgcaggcgtcatattctgatggacacgcaacacctgtctaaaactttgatagcccatgtaataccccggtataagcacaatgatatcggtcttgccggctaaagcactgtgcaatcgatggtagacaccgtactggtacgtcacgtcgtgaaatttcggcacacccccgtgagctccataccacgtaatcttcgatggcggtcggctcaatcttccaacgcctctcggagcatcaagttttagagaatccggtctcgccacgcatgaccgattgcccgaatcgggacatgcaactgccgatttcgcacacgaaagcacggaggctccgaagagcaccccgaccaactgaaacagaacgcatgatttagacccactcccgacagaggattaacgcgacccaatcaagggatatcaaaaaagaatccttaccgcggagagattcatagaccgaagagttgagagcgctcctgtttctctgaagatctctcaccccgactgtgacagatccacaaagcagcactcaatttatactgtcaaatggttaatgtttaatgctagaaaagcgctgacacccagtaaatatttacttagtttgcagttccactgtttccttattgccatgactggacaaaaaccacagataagatgttccattcaagggaacccgatgttccctcgataacttcccggtacaaagtccaaaaatagaactaggtgctttataaatactaagagtcgactccttggtgtttcagaagaacacagacgatctacaaacaggatgaacctcggaagactcaacaccgccggtaagaacatcttaatttttactttgtatgattttcaattctgaaaaacacgtttcctggttcgtgcacgtacgcggaaacgaagttcgaaaaatcagagttggaattttccagctatggttaactattaactatatgacgtcacttagttaattattaacgatataaagtaaatgattaactcgggctagttaatgattaactatacctggttaatgattaactgacttagttaatgattaactagaagttaatgattaactagaagttaatgattaactagaagttaatgattaatctattacgtcactcgttatatattaactagatttaaatgcggccgcgaattcactagtgattaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatccgatatcaaatgacttgccagacttacaacttgtttgttctgtctgtcatcatgatttattatggacatactgcaagtagtctaaatctccccattccagcccccggtacggaaacaggcgaaggagagggagagaccgagacaggcgaaggcgaaactggtgaagcaggtggcgaggaaggcgagcaaacaggagaaggcgaagtgcagcccccagaagaagagcttcctggggagagtgtaactgagcctgaggagaagcctgaggaggagctacctgaggaggaggttactgagcctgaggagaagcctgaggagggtgtgactcagcctgaggagacacctgagcagcctgttgagggtaccgaagaagagggcaagcaggagtctgaggctgcccccgaaactcctgccgtccagccaaaaccagaggagggtcacgaacgcccagaacccgaagaggaggaggagaagaaggaagaaggcggcggcttcccaacagctgcagtggcaggaggtgttggtggtgtgttgctcatagctgctgtaggtggtggtgttgcagccttcactagcggcggaggtggcgctggcgcacaggaggcagaacaggtcgagttcgaaggagaagataccggagcagcaactgccgagacacctgaagccgatacagttatcgacatcacagacgaagacgactactgggccgacagcggcgacattcagtgaagatctaggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctaatcgaattcccgcggccgctagtgacgtcatgagtaaatcattaaccttcatgcatatgcatgaggagctactgaatatgcatgagagcctcatacatatgcatggaacttatgcatattcacgacactcatgcatatgcatgcattggttaaagagtaaccctatgactcagtgtgtatgtttacgttgcctagcaacgttaatgatttacctgctgacgtggcagctccgcctccaggtaaatcatttacctgaactttgttctttatgtttattcaccatggcaacgctaccatatatggacatccgactccgcctcccccgttatacattaacgatggcgtgataggcggagctctcccccattggctctcaatgacgtagttcaggttaaccataagccagaaccgcctatataggtagagcaggtagacccggaacaccattcccatccggacctccatagagtgcggacctctacgggctctccataccggtaaatattttattccatttaatccaatcgaataaatcaataatcaactcaatgctgtgattctgcctcaaattcaatggtgattttctttaataaaaagcccaccccccttggcacccccctgtacacccccctgtacaggcgaccaccccctatggacacccccctgtacaggcgaccaccccctatggacacccccctgtacaggcgaccaccccctatggacacccccctgtacacccccctgtacaggcgaccaccccctatggacacccccctgtacaggcgaccaccccctatggacacccccctgtacacccccctgtacattttctcccataggctacaatggaatactgccccctagtgtctcctgctgtatgggacccctatgatgtgggcgccattacctttgccactatggagctccttcacgagggggcgccattgaaattgggagaccgcatagagagcctagccaatggggtgctttggaatccggatatccccgtccaactcttcaactgcctttccattcgctcatggggatcacatgggaagcgcgtcatgtaccgtggccacacctaccggatgtaccacgcccagttacgggtccgaagctccgcccccgttactaggaaacaggccggaagcctgctcctcagcctatcacagaagctcctctgtttcgccgcccgctttaatacccatcccctcgtgatgcaattgggggtggagtctaaccctatgggcctacctgtatataccaagagggccctccagatggcgctacagagtatgcgggtgcgcattgcccctgacggccagaaggtggcgccaccggagataggcaagacctgtacggtgaagcccctcaagaccccggagaccctccagcagggggtcttcagtaccaccgatttaaaaaagacacttccagattgggcttttcgccgactttttaaccaaaccccctatatttgtggatggaagattggcaccgcgccagaaggggcggagagttggatcgttacgctccacccccagccttcgactccgccccccacagggaccaagactccgcccactctgcaggaccttgcccggctgggcgtggtcgagcaatgcctcaagatgaggaggcgtggcctggaccgcaggcaccacccctatgctcaataaaccaatcagattccagtacttggctcctcctatttgtgggcgggactttgcacgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggtgcgggcccgagtcacggatggagtagtttcccttgcggccagcagagggcatacctttattctcagctcgcaagtctcaatagatacacacctcatcggtgtacagcgtgtccgcgtagcgcagccccgtgcacctcacccaaccacctatatcgcgaacggctccggtactcactatgtatttcccgacgcgatagttcggatcattgcaccacttattcaagtacattctaaaccattgcccttcggggacttggcgctgataaaaacattccctgaagtaccgtttcaccgcgcgagaacacttatacaagtatctgtcccgcaggttgaacatggttaagcacagaagcaaggtcatgtggcaggaacaagaaccgccaggctgcaaccccacgcagtatcccatcggatgaccgatctccgagttcgcctcctcgtggaacgggtaccatagctgcttcacgtcttgaaccagcttccagaacactgcatcgtgcatacaccacggcggcacggcaactccgaagatcacgtacagtggcatgttccggatacatctacgacacaggtactgtgacaccttgcgcgtacgcgaaaagggaacccgaccctcccccgtaacgctccacttacggacagccggcgatgcgcactgcgaacgaaaaataaatctgcgccgttgtgcgctccaggcggaaacaggggaatatataagccaactcttatctttattgttgccacgcccgacactatccagatttcgagacctgctgacaccaccggaacacgcgacctcgccctctctttatcatccatacgcccaggtgactcagtcaaatcccttatataaagaccgtttttacctgaccgcttccacgtacacaaggcggcacatgaaagcaccatgctgcgccccgtatccgccatcgcgttcctctcgtgcctatgtctcacgcggaccgcgcaacaggtcggtaagtcctttaatcttacgacccactccaacctcactgtgcacccgcagaccaaaggaacctcaaagcaagagtggcggctagggcgcgataccaagattgcgatgtgggagaaaggctacgggtacagctacccgtcgggaccctttaaaggccgcgtagaaatgaacgagaccagtgtcaccttttttgacctccgtcccaacgattctgccatactgacttacttctccgaagatagctccggcacggagagtgaatatccgtacgccatcagcgtaagaggtgagcccttccctacctttgttccattccgcccatcgagcacctcagccgacaccacacattttcagatcccctccgccctcccattctacggctgatgactaacaattcccgtccgcggaccgagagccgcatgagcttgcagtgcatcgcgctcgataacgatagttccattacgtacgcctggtacactgacaccttagagagcggggacaacatccgagaagtaaccgtccgaacggattctgaggtagcagttacctgtcggatatcggatggacattccaccaattccgcgactctcgtcgtgccgctaaaccgaggtcagtaatatcccctccctcaccgcaacagatccgcacagtcaggatcccaggctttcacgatcctttccccactcctttagaacctgccgctccctacggcgcggatatgactacggtgttcctggccatcttagcccttattcttctaaccgtcatcggcggctacgccctcagaaagctgtgtatgcgaaacgagcgcgtttttatttgtaacccgtacagagaatgttttggcggtcatctctaggacaaataaacttctacttgaaatgagtttatttttccccctgcctgtttgtgatgggaaatgatcggtgctgcttatggaccgagatagatggaagggacgggggcattcaaatttctaggtccagggacataaaaaagagatcaaatttacatctccggtaaagatcacctctataaccccgctgtgaatcccagcactcccttccgatacgcaaactgactagcagttcctgtgtatagacaaacggaatcctggtgtacagacaaacggaatcctgagttcccaacgcattcatttatttgaatatttacacatttacacactgtacacggtcattcgatttcattgccaacagaaagactaatcgatgtcccctttcagtatgtggactgtgacagcagggtcttcgctcacttcactgtccgtgtcatcctctgtacgcccacacagcatcgcccgatagtgaaagctgacactcagcatggagaaccaaacagcagggagcaatgtgagaggcagccaacacagggaaacttttttcttctcccttcagaccccctcccgtcgagacattgtgatggactactggtacttcgccctgatgttcccaggtaggaacgaccgtaagggtgaacctctgaacgctgttctgcatcaggtcccgtgtcacttgatacatgccggaatccgcgccacttaagttcttgatagtcacgcagttctcggatctgttatacgacagcctcccttgatacgccccgtagaccatcggctccttcagcgcctggtagtcctgcagcacgaacttgcgcacggcgcccgcatccaccgcggtcacttcccattctctgatctgaaaactctcggtagaaccgcacagagtcacagcgtcccgttcgttagccacgacccgggatacgttctccattttcaccgtaccgttctcgggaagccccgacacggtgaaatatacagtctcgggcttggaacccaccgcgaatgattgtgatagccagcacccgttattagctttgattgcttcgacatgcattgaattacaggatgaatttagccgcgccctcgtcatatagcccagatccagcccctctttgatcgaaggaatcacgaaagccactttctgccatctgttacagaccttccccggagcgtggtaccatagcagcagcgtgaaatcagcgcatctgcccgtagtcagagtcacctccttacggcccctcacggcggcaccggagacggtggcagacaagcagaggacggcggcacacagcaggagcgagccatgactgcggagtccgagccgagcggtgtgctgctcgatcctccgctacctttttatgcaccacccacctttattgtcggtcacacattaattcgccccgtcagcaaacacgtgagtaacgtatgccgttgttctgatcggtcagcaccgcgcccgcgacgtttgaacgaagacgtacggtgacttccgcatagggagctataaggaagtcagttaggaaaaatcgatcctcgacacaccccacggaaacgctgacctaggcgaaacctatcaggtaaaacattcactatacgcaccacgcgttgaataaacagttaacccatacggggtatatattctaccccgactgcagtcagcgatcaggacgcgtccacagagagatccgtgcgcgaccgcctgtccgggctcctctagaatcaagaatccataaccatgcaggtaagaactcctttctcttcctcctatttgatccgagcctttttttacgctactcaaccgcaacccgtttcttccaccacagttactgctccttctatgcctttgccctctgatgggcagcggaacactagcacccctcgtctcggtagacaactcctactccgtgttcggatccggcaaacccccactttctacctccgaatccgccaccaccgcctactccgaaaccgcggttcccgaaaactcttacccccatccgaagccaccacgccctgcgacgacttgctggaagaggactgctggttcgcggagagcagcgcggactacgcacccataccctggaacaccaaagagaatacgtccgtggttatcccggcacaggtagccgtctcgccatcgcagtccactactccccctgcggtcatgctcggcatcgcacagaaagccgtaaaccgcggagcctccagcaaggatcacacgtcccatatcgccacgggcgttaccgtagccggaatagtcatactcattgccctcgtcatcatagccttccgtacaaaggttaaggaaccgcgcccaacccgctccatctacctgggcgtgcctccccctgacgttagaccttaccgtataatagagcaataaagatttggccgccacatcgcacaagaatctttccgtgtcctgtgtctgtctcggcgccgtccgcgggaaaaggttaacgcggaatctatttccctgcggatttccgtatccgtcagttcctgggcgtcgccgaaaatgctcacggaagacacgcccatgcgggcgtggctaaccgatgattcgaaaaacgattcgcgagcgccctctgccggcggcggcgggaatagggggtgtggggggagtgtattttaagtagatatatatagatgatgctagag

SEQ ID NO: 18 - DNA, 84 bp, Artificial Sequence
  Secretion Signal of Gamma Interferon
atgacttgccagacttacaacttgtttgttctgtctgtcatcatgatttattatggacatactgcaagtagtctaaatctcccc

SEQ ID NO: 19 - protein, 28 aa, Artificial Sequence
  Secretion Signal of Gamma Interferon
MTCQTYNLFVLSVIMIYYGHTASSLNLP

SEQ ID NO: 20 - DNA, 636 bp, Eimeria maxima
attccagcccccggtacggaaacaggcgaaggagagggagagaccgagacaggcgaaggcgaaactggtgaagcaggtggcgaggaaggcgagcaaacaggagaaggcgaagtgcagcccccagaagaagagcttcctggggagagtgtaactgagcctgaggagaagcctgaggaggagctacctgaggaggaggttactgagcctgaggagaagcctgaggagggtgtgactcagcctgaggagacacctgagcagcctgttgagggtaccgaagaagagggcaagcaggagtctgaggctgcccccgaaactcctgccgtccagccaaaaccagaggagggtcacgaacgcccagaacccgaagaggaggaggagaagaaggaagaaggcggcggcttcccaacagctgcagtggcaggaggtgttggtggtgtgttgctcatagctgctgtaggtggtggtgttgcagccttcactagcggcggaggtggcgctggcgcacaggaggcagaacaggtcgagttcgaaggagaagataccggagcagcaactgccgagacacctgaagccgatacagttatcgacatcacagacgaagacgactactgggccgacagcggcgacattcagtga

SEQ ID NO: 21 - protein, 211 aa, Eimeria maxima
IPAPGTETGEGEGETETGEGETGEAGGEEGEQTGEGEVQPPEEELPGESVTEPEEKPEEELPEEEVTEPEEKPEEGVTQPEETPEQPVEGTEEEGKQESEAAPETPAVQPKPEEGHERPEPEEEEEKKEEGGGFPTAAVAGGVGGVLLIAAVGGGVAAFTSGGGGAGAQEAEQVEFEGEDTGAATAETPEADTVIDITDEDDYWADSGDIQ

SEQ ID NO: 22 - DNA, 13869 bp, Artificial Sequence
  Plasmid
cggatccctcgaggtcgacgaattcgagctcggccgacttggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttatgcatgcggccggccgcagctagcgtatctgtaatctcgactcatagtatacccattccaagtgctccggacaggacccactttgaaaccctcattaagcaagtgaaacatgggtaacccagttctcacatccaaagtctttagaaacttcggtactatcaatctatgaaaacagccgcttttactccacgtatccctccatgagaattcttcgcagaccgtctgtccccatacactttctgcacacactcttcccgaccatcccccatgaatcccacaacctctaccgcccttatcctccaacatgatatcctcagaagccgtataccctcctgtaatccacgacccttccgttttcaggactgccacgtaatccgcatcatttttatctaagcctccggtaacaaacgtgggctctaacccgactattctggtacaccgtctatccccagaaatatatgtccactgtctgcagatagtagagcaagcgtgagcgccggcagaatgtcctatacagtgtaaccttttcgaatgtggtatgtccgacagaaacttcccgaaatcaacatacagaccgtcatagtcgtagtacaggttccagtccaccaggagcagcgcgactgcaggcgtcatattctgatggacacgcaacacctgtctaaaactttgatagcccatgtaataccccggtataagcacaatgatatcggtcttgccggctaaagcactgtgcaatcgatggtagacaccgtactggtacgtcacgtcgtgaaatttcggcacacccccgtgagctccataccacgtaatcttcgatggcggtcggctcaatcttccaacgcctctcggagcatcaagttttagagaatccggtctcgccacgcatgaccgattgcccgaatcgggacatgcaactgccgatttcgcacacgaaagcacggaggctccgaagagcaccccgaccaactgaaacagaacgcatgatttagacccactcccgacagaggattaacgcgacccaatcaagggatatcaaaaaagaatccttaccgcggagagattcatagaccgaagagttgagagcgctcctgtttctctgaagatctctcaccccgactgtgacagatccacaaagcagcactcaatttatactgtcaaatggttaatgtttaatgctagaaaagcgctgacacccagtaaatatttacttagtttgcagtgccactgtttccatattgccatgactggacaaaaaccacagataagatgtgccattcaagggaacccgatgttccctcgataacttcccggtacaaagtccaaaaatagaactaggtgctttataaatactaagagtcgactccttggtgtttcagaagaacacagacgatctacaaacaggatgaacctcggaagactcaacaccgccggtaagaacatcttaatttttactttgtatgattttcaattctgaaaaacacgtttcctggttcgtgcacgtacgcggaaacgaagttcgaaaaatcagagttggaattttccagctatggttaactattaactatatgacgtcacttagttaattattaacgatataaagtaaatgattaactcgggctagttaatgattaactatacctggttaatgattaactgacttagttaatgattaactagaagttaatgattaactagaagttaatgattaactagaagttaatgattaatctattacgtcactcgttatatattaactagatttaaatgcggccgctgctcgtgaccagcccaaaacaaagcatgctatttgggtggcataacgtttgttttcgacttgtttgtccaggctttctaggtggagtacggtgagcgcctccggtggcgcgtcgaggaatcgaacgggcttgaatgcggtctcggtggctcgcgagtgggcggggtttgtttctgccggcggtcgcccgtcatctatataaaggccgcaggtgagcgcttcttccagctcctgatcgacttcggagaggtctgcctcctcggcggatcctgtccgagccatcccgcttgaggatcgttttcgaccgcgcggacgagccgctgagtgtctagctcgccaaaggcttcgacgaagaggttgagccaatcgtcttcagcgaacacttccgatccaggtgatgtagatatcaaatgacttgccagacttacaacttgtttgttctgtctgtcatcatgatttattatggacatactgcaagtagtctaaatcttaccactcctgttgagaaccaggttcacccttacagcgagatgagtacctaccaggaggggagtgccccgggggctccggaggacaccaccaccaccactacgtcgtcccctgtttccgatggagccgagcgttggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccagggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaacttaaagatcttattaaagcagaacttgtttattgcagcttataatggttacaaataaagcaatagcatcacaaatttcacaaataaagcatttttttcactgcattctagttgtggtttgtccaaactcatcaatgtatctaatcatgtctggtcgacgcggccgctagtgacgtcatgagtaaatcattaaccttcatgcatatgcatgaggagctactgaatatgcatgagagcctcatacatatgcatggaacttatgcatattcacgacactcatgcatatgcatgcattggttaaagagtaaccctatgactcagtgtgtatgtttacgttgcctagcaacgttaatgatttacctgctgacgtggcagctccgcctccaggtaaatcatttacctgaactttgttctttatgtttattcaccatggcaacgctaccatatatggacatccgactccgcctcccccgttatacattaacgatggcgtgataggcggagctctcccccattggctctcaatgacgtagttcaggttaaccataagccagaaccgcctatataggtagagcaggtagacccggaacaccattcccatccggacctccatagagtgcggacctctacgggctctccataccggtaaatattttattccatttaatccaatcgaataaatcaataatcaactcaatgctgtgattctgcctcaaattcaatggtgattttctttaataaaaagcccaccccccttggcacccccctgtacacccccctgtacaggcgaccaccccctatggacacccccctgtacaggcgaccaccccctatggacacccccctgtacaggcgaccaccccctatggacacccccctgtacacccccctgtacaggcgaccaccccctatggacacccccctgtacaggcgaccaccccctatggacacccccctgtacacccccctgtacattttctcccataggctacaatggaatactgccccctagtgtctcctgctgtatgggacccctatgatgtgggcgccattacctttgccactatggagctccttcacgagggggcgccattgaaattgggagaccgcatagagagcctagccaatggggtgctttggaatccggatatccccgtccaactcttcaactgcctttccattcgctcatggggatcacatgggaagcgcgtcatgtaccgtggccacacctaccggatgtaccacgcccagttacgggtccgaagctccgcccccgttactaggaaacaggccggaagcctgctcctcagcctatcacagaagctcctctgtttcgccgcccgctttaatacccatcccctcgtgatgcaattgggggtggagtctaaccctatgggcctacctgtatataccaagagggccctccagatggcgctacagagtatgcgggtgcgcattgcccctgacggccagaaggtggcgccaccggagataggcaagacctgtacggtgaagcccctcaagaccccggagaccctccagcagggggtcttcagtaccaccgatttaaaaaagacacttccagattgggcttttcgccgactttttaaccaaaccccctatatttgtggatggaagattggcaccgcgccagaaggggcggagagttggatcgttacgctccacccccagccttcgactccgccccccacagggaccaagactccgcccactctgcaggaccttgcccggctgggcgtggtcgagcaatgcctcaagatgaggaggcgtggcctggaccgcaggcaccacccctatgctcaataaaccaatcagattccagtacttggctcctcctatttgtgggcgggactttgcacgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggcgcggggccggtcaatcattaacccgacggccggcacgggcgccccctggcggcgggcgcccgccactgcaccctgcgcctcttagcggcgccccctggcggccgagggccgccactgcacccctgtcggacttagtctctggtgcgggcccgagtcacggatggagtagtttcccttgcggccagcagagggcatacctttattctcagctcgcaagtctcaatagatacacacctcatcggtgtacagcgtgtccgcgtagcgcagccccgtgcacctcacccaaccacctatatcgcgaacggctccggtactcactatgtatttcccgacgcgatagttcggatcattgcaccacttattcaagtacattctaaaccattgcccttcggggacttggcgctgataaaaacattccctgaagtaccgtttcaccgcgcgagaacacttatacaagtatctgtcccgcaggttgaacatggttaagcacagaagcaaggtcatgtggcaggaacaagaaccgccaggctgcaaccccacgcagtatcccatcggatgaccgatctccgagttcgcctcctcgtggaacgggtaccatagctgcttcacgtcttgaaccagcttccagaacactgcatcgtgcatacaccacggcggcacggcaactccgaagatcacgtacagtggcatgttccggatacatctacgacacaggtactgtgacaccttgcgcgtacgcgaaaagggaacccgaccctcccccgtaacgctccacttacggacagccggcgatgcgcactgcgaacgaaaaataaatctgcgccgttgtgcgctccaggcggaaacaggggaatatataagccaactcttatctttattgttgccacgcccgacactatccagatttcgagacctgctgacaccaccggaacacgcgacctcgccctctctttatcatccatacgcccaggtgactcagtcaaatcccttatataaagaccgtttttacctgaccgcttccacgtacacaaggcggcacatgaaagcaccatgctgcgccccgtatccgccatcgcgttcctctcgtgcctatgtctcacgcggaccgcgcaacaggtcggtaagtcctttaatcttacgacccactccaacctcactgtgcacccgcagaccaaaggaacctcaaagcaagagtggcggctagggcgcgataccaagattgcgatgtgggagaaaggctacgggtacagctacccgtcgggaccctttaaaggccgcgtagaaatgaacgagaccagtgtcaccttttttgacctccgtcccaacgattctgccatactgacttacttctccgaagatagctccggcacggagagtgaatatccgtacgccatcagcgtaagaggtgagcccttccctacctttgttccattccgcccatcgagcacctcagccgacaccacacattttcagatcccctccgccctcccattctacggctgatgactaacaattcccgtccgcggaccgagagccgcatgagcttgcagtgcatcgcgctcgataacgatagttccattacgtacgcctggtacactgacaccttagagagcggggacaacatccgagaagtaaccgtccgaacggattctgaggtagcagttacctgtcggatatcggatggacattccaccaattccgcgactctcgtcgtgccgctaaaccgaggtcagtaatatcccctccctcaccgcaacagatccgcacagtcaggatcccaggctttcacgatcctttccccactcctttagaacctgccgctccctacggcgcggatatgactacggtgttcctggccatcttagcccttattcttctaaccgtcatcggcggctacgccctcagaaagctgtgtatgcgaaacgagcgcgtttttatttgtaacccgtacagagaatgttttggcggtcatctctaggacaaataaacttctacttgaaatgagtttatttttccccctgcctgtttgtgatgggaaatgatcggtgctgcttatggaccgagatagatggaagggacgggggcattcaaatttctaggtccagggacataaaaaagagatcaaatttacatctccggtaaagatcacctctataaccccgctgtgaatcccagcactcccttccgatacgcaaactgactagcagttcctgtgtatagacaaacggaatcctggtgtacagacaaacggaatcctgagttcccaacgcattcatttatttgaatatttacacatttacacactgtacacggtcattcgatttcattgccaacagaaagactaatcgatgtcccctttcagtatgtggactgtgacagcagggtcttcgctcacttcactgtccgtgtcatcctctgtacgcccacacagcatcgcccgatagtgaaagctgacactcagcatggagaaccaaacagcagggagcaatgtgagaggcagccaacacagggaaacttttttcttctcccttcagaccccctcccgtcgagacattgtgatggactactggtacttcgccctgatgttcccaggtaggaacgaccgtaagggtgaacctctgaacgctgttctgcatcaggtcccgtgtcacttgatacatgccggaatccgcgccacttaagttcttgatagtcacgcagttctcggatctgttatacgacagcctcccttgatacgccccgtagaccatcggctccttcagcgcctggtagtcctgcagcacgaacttgcgcacggcgcccgcatccaccgcggtcacttcccattctctgatctgaaaactctcggtagaaccgcacagagtcacagcgtcccgttcgttagccacgacccgggatacgttctccattttcaccgtaccgttctcgggaagccccgacacggtgaaatatacagtctcgggcttggaacccaccgcgaatgattgtgatagccagcacccgttattagctttgattgcttcgacatgcattgaattacaggatgaatttagccgcgccctcgtcatatagcccagatccagcccctctttgatcgaaggaatcacgaaagccactttctgccatctgttacagaccttccccggagcgtggtaccatagcagcagcgtgaaatcagcgcatctgcccgtagtcagagtcacctccttacggcccctcacggcggcaccggagacggtggcagacaagcagaggacggcggcacacagcaggagcgagccatgactgcggagtccgagccgagcggtgtgctgctcgatcctccgctacctttttatgcaccacccacctttattgtcggtcacacattaattcgccccgtcagcaaacacgtgagtaacgtatgccgttgttctgatcggtcagcaccgcgcccgcgacgtttgaacgaagacgtacggtgacttccgcatagggagctataaggaagtcagttaggaaaaatcgatcctcgacacaccccacggaaacgctgacctaggcgaaacctatcaggtaaaacattcactatacgcaccacgcgttgaataaacagttaacccatacggggtatatattctaccccgactgcagtcagcgatcaggacgcgtccacagagagatccgtgcgcgaccgcctgtccgggctcctctagaatcaagaatccataaccatgcaggtaagaactcctttctcttcctcctatttgatccgagcctttttttacgctactcaaccgcaacccgtttcttccaccacagttactgctccttctatgcctttgccctctgatgggcagcggaacactagcacccctcgtctcggtagacaactcctactccgtgttcggatccggcaaacccccactttctacctccgaatccgccaccaccgcctactccgaaaccgcggttcccgaaaactcttacccccatccgaagccaccacgccctgcgacgacttgctggaagaggactgctggttcgcggagagcagcgcggactacgcacccataccctggaacaccaaagagaatacgtccgtggttatcccggcacaggtagccgtctcgccatcgcagtccactactccccctgcggtcatgctcggcatcgcacagaaagccgtaaaccgcggagcctccagcaaggatcacacgtcccatatcgccacgggcgttaccgtagccggaatagtcatactcattgccctcgtcatcatagccttccgtacaaaggttaaggaaccgcgcccaacccgctccatctacctgggcgtgcctccccctgacgttagaccttaccgtataatagagcaataaagatttggccgccacatcgcacaagaatctttccgtgtcctgtgtctgtctcggcgccgtccgcgggaaaaggttaacgcggaatctatttccctgcggatttccgtatccgtcagttcctgggcgtcgccgaaaatgctcacggaagacacgcccatgcgggcgtggctaaccgatgattcgaaaaacgattcgcgagcgccctctgccggcggcggcgggaatagggggtgtggggggagtgtattttaagtagatatatatagatgatgctagag

SEQ ID NO: 23 - DNA, 81 bp, Artificial Sequence
  Secretion Signal Sequence
atgacttgccagacttacaacttgtttgttctgtctgtcatcatgatttattatggacatactgcaagtagtctaaatctt

SEQ ID NO: 24 - DNA, 969 bp, Eimeria maxima
accactcctgttgagaaccaggttcacccttacagcgagatgagtacctaccaggaggggagtgccccgggggctccggaggacaccaccaccaccactacgtcgtcccctgtttccgatggagccgagcgttggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccagggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaacttaa

SEQ ID NO: 25 - protein, 322 aa, Eimeria maxima
TTPVENQVHPYSEMSTYQEGSAPGAPEDTTTTTTSSPVSDGAERWLESFVRAVQRQLQLQDQMMRQLMRDIQEYLSTAFNWAENQSTAYTRVTEMMDMISNRMNAARDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFYYGGYPSYYNSPYYSYSSYPSYYNYSYPSYSYSSYPSYYRYSSYPYYNYSYPSYYNYGSYPYYSYSSYPSWYWRRLRSLATATCPDCPPLTTPSMIPT

SEQ ID NO: 26 - DNA, 1515 bp, Eimeria maxima
atacaaatcctttttatctggttccaacacgctcactcaaccaccacctggacacaccctccccatacatacaggagcagcagcaacaccagcatcaagatgacgcgtgcggcagcgcttgccggggttttggccctggctgcagcaggcagcagccttgctctacctactgtattggacacaacgactggcacccaagtggagtggactgagacccccttagacacaacagaggtaactatgggggagatgggcagcaccaccagcggcacgactccaaccagcactggtgtgcgaatgatggaggctgaaactacaaccccatcaacccctgaggctccccagcagcagcagcagatgcctcagcctcaacctcagccacagcaaacaactcccgttcctgaggccgtattagaggcaattatgcaagaaatgcaaaatattttccgttcttctcttgtaccaggttgggatactgtcggtacagcagcagatgctgtacgtcagattgtaacccgtgtaagagaacgtcttacaggaccattaatgatgacagagatggatactggtcttggtagaacaggacctttatcaaccacaggtgcaacaggagcaacaacaggtcctgttgctgcattacgcggtgtaacaaatgatttccttagggaaataatgattcaagaagcagtacttgagacattatgggcagttgtacgtgatgcacaagaaagaccatggctagttaatgaacaggaagtattgcatgcagtaacagcagatgctgtacaaggtttccttggtcgcatgcatgatcgtcttcgtgcaacaggtttctctgaggaagaagtcatgagacttctacctaggtcacgtaatggtggttgtacccgtacagggggcctctttgatcaatgtaacgatgcccctccctctcgtcttcttggtaagaggatgtatagtactggatattatggttatggatatccttcttattatagctatggatatagttatccagcttattcacattatcctgtttcttatccttactatgggtatagctggggcccctcatactactatggcagcggatactatggtaaacatggatataagtacggagtaggagaaagatatgagcctattacccctacacaaagaactttttataataatacagaaggtactaacaaccctgtctatacacccgaaaatcttacagaagatgaaccacaaactgtatgggaaacatacaactaaaccctaaaccctaaaccctaaaccctcaaccctaacatttctcatttttttatagagaaattttagggaacactaacctgcctgccttgccatcgtttatatatatccatttgtttattaataaacaatttttatttacctctagtcgtctttttattaacagcgcttattcgcgttgtttatacaaactactactatttttacccaataatacttgtacaggcattttttaaaaaaaaaaaaaaaaaaaaaaaa

SEQ ID NO: 27 - DNA, 1515 bp, Eimeria maxima
tatgtttaggaaaaatagaccaaggttgtgcgagtgagttggtggtggacctgtgtgggaggggtatgtatgtcctcgtcgtcgttgtggtcgtagttctactgcgcacgccgtcgcgaacggccccaaaaccgggaccgacgtcgtccgtcgtcggaacgagatggatgacataacctgtgttgctgaccgtgggttcacctcacctgactctgggggaatctgtgttgtctccattgataccccctctacccgtcgtggtggtcgccgtgctgaggttggtcgtgaccacacgcttactacctccgactttgatgttggggtagttggggactccgaggggtcgtcgtcgtcgtctacggagtcggagttggagtcggtgtcgtttgttgagggcaaggactccggcataatctccgttaatacgttctttacgttttataaaaggcaagaagagaacatggtccaaccctatgacagccatgtcgtcgtctacgacatgcagtctaacattgggcacattctcttgcagaatgtcctggtaattactactgtctctacctatgaccagaaccatcttgtcctggaaatagttggtgtccacgttgtcctcgttgttgtccaggacaacgacgtaatgcgccacattgtttactaaaggaatccctttattactaagttcttcgtcatgaactctgtaatacccgtcaacatgcactacgtgttctttctggtaccgatcaattacttgtccttcataacgtacgtcattgtcgtctacgacatgttccaaaggaaccagcgtacgtactagcagaagcacgttgtccaaagagactccttcttcagtactctgaagatggatccagtgcattaccaccaacatgggcatgtcccccggagaaactagttacattgctacggggagggagagcagaagaaccattctcctacatatcatgacctataataccaatacctataggaagaataatatcgatacctatatcaataggtcgaataagtgtaataggacaaagaataggaatgatacccatatcgaccccggggagtatgatgataccgtcgcctatgataccatttgtacctatattcatgcctcatcctctttctatactcggataatggggatgtgtttcttgaaaaatattattatgtcttccatgattgttgggacagatatgtgggcttttagaatgtcttctacttggtgtttgacataccctttgtatgttgatttgggatttgggatttgggatttgggagttgggattgtaaagagtaaaaaaatatctctttaaaatcccttgtgattggacggacggaacggtagcaaatatatataggtaaacaaataattatttgttaaaaataaatggagatcagcagaaaaataattgtcgcgaataagcgcaacaaatatgtttgatgatgataaaaatgggttattatgaacatgtccgtaaaaaatttttttttttttttttttttttt

SEQ ID NO: 28 - protein, 386 aa, Eimeria maxima
MTRAAALAGVLALAAAGSSLALPTVLDTTTGTQVEWTETPLDTTEVTMGEMGSTTSGTTPTSTGVRMMEAETTTPSTPEAPQQQQQMPQPQPQPQQTTPVPEAVLEAIMQEMQNIFRSSLVPGWDTVGTAADAVRQIVTRVRERLTGPLMMTEMDTGLGRTGPLSTTGATGATTGPVAALRGVTNDFLREIMIQEAVLETLWAVVRDAQERPWLVNEQEVLHAVTADAVQGFLGRMHDRLRATGFSEEEVMRLLPRSRNGGCTRTGGLFDQCNDAPPSRLLGKRMYSTGYYGYGYPSYYSYGYSYPAYSHYPVSYPYYGYSWGPSYYYGSGYYGKHGYKYRVGERYEPITPTQRTFYNNTEGTNNPVYTPENLTEDEPQTVWETYN

SEQ ID NO: 29 - protein, 335 aa, Eimeria tenella; identical to SEQ ID NO: 12

SEQ ID NO: 30 - protein, 322 aa, Eimeria maxima
TTPVENQVHPYSEMSTYQEGSAPGAPEDTTTTTTSSPVSDGAEQWLESFVRAVQRQLQLQDQMMRQLMRDIQEYLSTAFNWAENQSTAYTRVTEMMDMISNRMNAAMDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFYYGGYPSYYNSPYYSYSSYPSYYNYSYPSYSYSSYPSYYRYSSYPYYNYSYPSYYNYGSYPYYSYSSYPSWYWRRLRSLATATCPDCPPLTTPSMIPT

SEQ ID NO: 31 - protein, 249 aa, Eimeria tenella
MTRLSLCALAVALAVGQSLAVPTTVENTVHPYSEMGTYQEGEAPGAPDESSTTTTTPSPSPEAPDQWLENFVRAVQRQLQLQESMMRQLVKEIQEYLSRAFNWDENQSAAYNRVNEMMDMITNRMTTALDGANELMATSETMDPETLRRATRKYMKEVRVQDVVVDSLWASLRGGVQTSAWMSGVTAVEKEETTPMAGRAAEEFMHRMYHNLRAAGMAEEDITRFMPKTEYTTPREQTRNMGRKGRYGY

SEQ ID NO: 32 - protein, 248 aa, Eimeria maxima
MTRLGLAAVALALAVGPSMAVPSTTPVENQVHPYSEMSTYQEGSAPGAPEDTTTTTTSSPVSDGAEQWLESFVRAVQRQLQLQDQMMRQLMRDIQEYLSTAFNWAENQSTAYTRVTEMMDMISNRMNAAMDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFY

SEQ ID NO: 33 - protein, 225 aa, Eimeria tenella
VENTVHPYSEMGTYQEGEAPGAPDESSTTTTTPSPSPEAPDQWLENFVRAVQRQLQLQESMMRQLVKEIQEYLSRAFNWDENQSAAYNRVNEMMDMITNRMTTALDGANELMATSETMDPETLRRATRKYMKEVRVQDVVVDSLWASLRGGVQTSAWMSGVTAVEKEETTPMAGRAAEEFMHRMYHNLRAAGMAEEDITRFMPKTEYTTPREQTRNMGRKGRYGY

SEQ ID NO: 34 - protein, 222 aa, Eimeria maxima
VENQVHPYSEMSTYQEGSAPGAPEDTTTTTTSSPVSDGAEQWLESFVRAVQRQLQLQDQMMRQLMRDIQEYLSTAFNWAENQSTAYTRVTEMMDMISNRMNAAMDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFY

SEQ ID NO: 35 - protein, 29 aa, Rattus norvegicus
MRCFISLVLGLLALEVALARNLQEHVFNS

SEQ ID NO: 36 - protein, 28 aa, Rattus norvegicus
MALHMVLVVLSLLPLLEAQNPEPANITL

SEQ ID NO: 37 - protein, 34 aa, Mus musculus
MDYYRKYAAVILVMLSMFLHILHSLPDGDFIIQG

SEQ ID NO: 38 - protein, 36 aa, Myxine glutinosa
MALSPFLAAVIPLVLLLSRAPPSADTRTTGHLCGKD

SEQ ID NO: 39 - protein, 34 aa, Lophius piscatorius
MAALWLQSFSLLVLLVVSWPGSQAVAPAQHLCGS

SEQ ID NO: 40 - protein, 34 aa, Homo Sapiens
MALWMRLLPLLALLALWGPDPAAAFVNQHLCGSH

SEQ ID NO: 41 - protein, 34 aa, Rattus norvegicus
MALWMRFLPLLALLVLWEPKPAQAFVKQHLCGPH

SEQ ID NO: 42 - protein, 34 aa, Rattus norvegicus
MALWIRFLPLLALLILWEPRPAQAFVKQHLCGSH

SEQ ID NO: 43 - protein, 25 aa, Ovis aries
MKVLILACLVALALAREQEELNVVG

SEQ ID NO: 44 - protein, 31 aa, Ovis aries
MRKSILLVVTILALTLPFLIAQEQNQEQRIC

SEQ ID NO: 45 - protein, 29 aa, Ovis aries
MMSFVSLLLVGILFWATQAEQLTKCEVFQ

SEQ ID NO: 46 - protein, 28 aa, Ovis aries
MKCLLLALGLALACGVQAIIVTQTMKGL

SEQ ID NO: 47 - protein, 25 aa, Ovis aries
  misc_feature (24)..(24): Xaa can be any naturally occurring amino acid
MKLLILTCLVAVALARPKHPIKHXG

SEQ ID NO: 48 - protein, 25 aa, Ovis aries
MKVLMKACLVAVALAKNTMEHVSSS

SEQ ID NO: 49 - protein, 26 aa, VS Virus
  misc_feature (24)..(26): Xaa can be any naturally occurring amino acid
MKCLLYLAFLFIHVNCKFTIVFPXXX

SEQ ID NO: 50 - protein, 33 aa, Gallus gallus
MQYRALVIAVILLLSTTVPEVCSKSIIDRERRD

SEQ ID NO: 51 - protein, 31 aa, Apis mellifera
MKFLVNVALVFMVVYISYIYAAPEPEPAPEP

SEQ ID NO: 52 - protein, 39 aa, Rattus norvegicus
  misc_feature (31)..(32): Xaa can be any naturally occurring amino acid
MNSQVSARKAGTLLLLMMSNLLFCQNVQTLXXCXXXXCX

SEQ ID NO: 53 - protein, 35 aa, Homo Sapiens
MPGSRTSLLLAFALLCLPWLQEAGAVQTVPLSRLF

SEQ ID NO: 54 - protein, 30 aa, Homo Sapiens
MEMFQGLLLLLLLSMGGTWASKEPLRPRCR

SEQ ID NO: 55 - protein, 34 aa, Homo Sapiens
MDYYRKYAAIFLVTLSVFLHVLHSAPDVQDCPEC

SEQ ID NO: 56 - protein, 31 aa, Oryctolagus cuniculus
  misc_feature (29)..(29): Xaa can be any naturally occurring amino acid
MKLAITLALVTLALLCSPASAGICPRFAXVI

SEQ ID NO: 57 - protein, 36 aa, Rattus norvegicus
MAADSQTPWLLTFSLLCLLWPQEAGALPAMPLSSLF

SEQ ID NO: 58 - protein, 36 aa, Homo Sapiens
MATGSRTSLLLAFGLLCLPWLQEGSAFPTIPLSRLF

SEQ ID NO: 59 - protein, 37 aa, Bos taurus
MMAAGPRTSLLLAFALLCLPWTQVVGAFPAMSLSGLF

SEQ ID NO: 60 - protein, 35 aa, Bos taurus
MMSAKDMVKVMIVMLAICFLARSDGKSVKKRAVSE

SEQ ID NO: 61 - protein, 32 aa, Rattus norvegicus
MSSRLLLQLLGFWLFLSQPCRARVSEEWMDQV

SEQ ID NO: 62 - protein, 28 aa, Rattus norvegicus
MKWVTFLLLLFISGSAFSRGVFRREAHK

SEQ ID NO: 63 - protein, 28 aa, Homo Sapiens
MKWVTFISLLFLFSSAYSRGVFRRDAHK

SEQ ID NO: 64 - protein, 28 aa, Rattus norvegicus
MKWVTFLLLLFISGSAFSRGVFRREAHK

SEQ ID NO: 65 - protein, 34 aa, Gallus gallus
MRQAAAPLLPGVLLLFSILPASQQGGVPGAIPGG

SEQ ID NO: 66 - protein, 34 aa, Gallus gallus
  misc_feature (32)..(34): Xaa can be any naturally occurring amino acid
MAMAGVFVLFSFVLCGFLPDAAFGAEVDCSRXXX

SEQ ID NO: 67 - protein, 28 aa, Gallus gallus
  misc_feature (26)..(28): Xaa can be any naturally occurring amino acid
MRSLLILVLCFLPLAALGKVFGRCEXXX

SEQ ID NO: 68 - protein, 29 aa, Gallus gallus
  misc_feature (27)..(29): Xaa can be any naturally occurring amino acid
MKLILCTVLSLGIAAVCFAAPPKSVIXXX

SEQ ID NO: 69 - protein, 34 aa, Homo Sapiens
MPSSVSWGILLLAGLCCLVPVSLAEDPQGDAAQK

SEQ ID NO: 70 - protein, 33 aa, Rattus norvegicus
MSTVELSLCLLIMLAVCCYEANASQICELVAHE

SEQ ID NO: 71 - protein, 30 aa, Rattus norvegicus
MRLSLCLLTILVVCCYEANGQTLAGVCQAL

SEQ ID NO: 72 - protein, 27 aa, AD Virus
MRYMILGLLALAAVCSAAKKVEFKEPA

SEQ ID NO: 73 - protein, 28 aa, Rattus norvegicus
  misc_feature (16)..(17): Xaa can be any naturally occurring amino acid
MKAAVLAVALVFLTGXXAXEFXXXDEPX

SEQ ID NO: 74 - protein, 29 aa, Rabies virus
MVPQALLFVPLLVFPLCFGKFPIYTILDK

SEQ ID NO: 75 - protein, 26 aa, Influenza virus
MKTIIALSYIFCLVFAQDLPGNDNNS

SEQ ID NO: 76 - protein, 25 aa, Influenza virus
MAIIYLILLFTAVRGDQICIGYHAN

SEQ ID NO: 77 - protein, 28 aa, Influenza virus
MNTQILVFALVAVIPTNADKICLGHHAV

SEQ ID NO: 78 - protein, 33 aa, Homo Sapiens
MALTFALLVALLVLSCKSSCSVGCDLPQTHSLG

SEQ ID NO: 79 - protein, 33 aa, Homo Sapiens
MALTFYLMVALVVLSYKSFSSLGCDLPQTHSLG

SEQ ID NO: 80 - protein, 33 aa, Homo Sapiens
MALSFSLLMAVLVLSYKSICSLGCDLPQTHSLG

SEQ ID NO: 81 - protein, 33 aa, Homo Sapiens
MASPFALLMVLVVLSCKSSCSLGCDLPETHSLG

SEQ ID NO: 82 - protein, 33 aa, Homo Sapiens; identical to SEQ ID NO: 80

SEQ ID NO: 83 - protein, 33 aa, Homo Sapiens
MALPFSLMMALVVLSCKSSCSLGCNLSQTHSLN

SEQ ID NO: 84 - protein, 33 aa, Homo Sapiens
MALPFALMMALVVLSCKSSCSLGCNLSQTHSLN

SEQ ID NO: 85 - protein, 30 aa, Homo Sapiens
MKYTSYILAFQLCIVLGSLGCYCQDPYVKE

SEQ ID NO: 86 - protein, 31 aa, Homo Sapiens
MTNKCLLQIALLLCFSTTALSMSYNLLGFLQ

SEQ ID NO: 87 - protein, 30 aa, Mus musculus
MRAPAQIFGFLLLLFPGTRCDIQMTQSPSS

SEQ ID NO: 88 - protein, 30 aa, Mus musculus
METDTLLLWVLLLWVPGSTGDIVLTQSPAS

SEQ ID NO: 89 - protein, 30 aa, Mus musculus
METDTLLLWVLLLWVPGSTGNIVLTQSPAS

SEQ ID NO: 90 - protein, 30 aa, Mus musculus
METDTLLLWVLLLWVPGADAAPTVSIFPPS

SEQ ID NO: 91 - protein, 29 aa, Mus musculus
MAWISLILSLLALSSGAISQAVVTQESAL

SEQ ID NO: 92 - protein, 29 aa, Mus musculus
MAWISLILSLLALSSGAISQAVVTQESAL

SEQ ID NO: 93 - protein, 29 aa, Mus musculus
MAWTSLILSLLALCSGASSQAVVTQESAL

SEQ ID NO: 94 - protein, 34 aa, Mus musculus
  misc_feature (28)..(34): Xaa can be any naturally occurring amino acid
MGVRMESHTRVFIFLLLWLSGTDGDIVXXXXXXX

SEQ ID NO: 95 - protein, 32 aa, Mus musculus
MDMRAPAQIFGFLLLLFPGTRCDIQMTQSPSS

SEQ ID NO: 96 - protein, 28 aa, Mus musculus
MKVLSLLYLLTAIPGIMSDVQLQESGPG

SEQ ID NO: 97 - protein, 29 aa, Mus musculus
  misc_feature (20)..(29): Xaa can be any naturally occurring amino acid
MGWSWIFLFLLSGTAGVHSXXXXXXXXXX

SEQ ID NO: 98 - protein, 29 aa, Mus musculus
  misc_feature (20)..(29): Xaa can be any naturally occurring amino acid
IKWSWISLFLLSGTAGVHSXXXXXXXXXX

SEQ ID NO: 99 - protein, 29 aa, Mus musculus
  misc_feature (20)..(29): Xaa can be any naturally occurring amino acid
MECSWVFLFLLSLTAGIHCXXXXXXXXXX

SEQ ID NO: 100 - protein, 29 aa, Mus musculus
  misc_feature (20)..(29): Xaa can be any naturally occurring amino acid
MEWSGVFIFLLSVTAGVYSXXXXXXXXXX

SEQ ID NO: 101 - protein, 29 aa, Mus musculus
MGWSFIFLFLLSVTAGVHSEVQLQQSGAE

SEQ ID NO: 102 - protein, 25 aa, Canis lupus
  misc_feature (1)..(2): Xaa can be any naturally occurring amino acid
XXPLLILAFLXAAVATPTDDDDKIV

SEQ ID NO: 103 - protein, 25 aa, Canis lupus
  misc_feature (3)..(3): Xaa can be any naturally occurring amino acid
ALXIIFLALLXAXVAFPIDDDDKIV

SEQ ID NO: 104 - protein, 27 aa, Canis lupus
  misc_feature (7)..(7): Xaa can be any naturally occurring amino acid
AFLILVXAFALXXVAFXXXVPAIXPVX

SEQ ID NO: 105 - protein, 26 aa, Canis lupus
  misc_feature (1)..(2): Xaa can be any naturally occurring amino acid
XXLILVFGALLXAIYXQXAFVXXXXX

SEQ ID NO: 106 - protein, 25 aa, Canis lupus
  misc_feature (1)..(2): Xaa can be any naturally occurring amino acid
XXFFLLLXVIGFXVAQYAPHXXXXX

SEQ ID NO: 107 - protein, 25 aa, Mus musculus
MKFFLLLSLIGFCWAQYDPHTQYGR

SEQ ID NO: 108 - protein, 25 aa, Mus musculus
MKFVLLLSLIGFCWAQYDPHTSDGR

SEQ ID NO: 109 - protein, 20 aa, Rattus norvegicus
LLSLIGFCYAQYDPHTADGR

SEQ ID NO: 110 - protein, 29 aa, Oryctolagus cuniculus
MMPLVPLLLVSIVFPGIQATQLTRCELTE

SEQ ID NO: 111 - protein, 29 aa, Sus sofra
MMSFVSLLVVGILFPAIQAKQFTKCELSQ

SEQ ID NO: 112 - protein, 26 aa, Rattus norvegicus
MKRLLILSLLLEAVCGNENFVGHQVL

SEQ ID NO: 113 - protein, 36 aa, Bos taurus
MPRLCSSRSGALLLALLLQASMEVRGWCLESSQCQD

SEQ ID NO: 114 - protein, 30 aa, Sus sofra
MAWQGLLLAACLLVLPSTMADCLSGCSLCA

SEQ ID NO: 115 - protein, 30 aa, Homo Sapiens
MARFLTLCTWLLLLGPGLLATVRAECSQDC

SEQ ID NO: 116 - protein, 31 aa, Sus sofra
  misc_feature (30)..(31): Xaa can be any naturally occurring amino acid
MQRLCAYVLIHVLALAACSEASWKPGFQLXX

SEQ ID NO: 117 - protein, 31 aa, Mus musculus
MDRRRMPLWALLLLWSPCTFSLPTGTTFERI

SEQ ID NO: 118 - protein, 31 aa, Trypanosome
MVKAIASLMLLHIWAIEEIKAERQAPSVSRT

SEQ ID NO: 119 - protein, 33 aa, Ictalurus punctatus
MSSSPLRLALALMCLVSAVGVISCGRPHVVLNS

SEQ ID NO: 120 - protein, 36 aa, Lophius piscatorius
MVSSSRLRCLLVLLLSLTVSISCSFAGQRDSKLRLL

SEQ ID NO: 121 - protein, 38 aa, Lophius piscatorius
MKMVSSSRLRCLLVLLLSLTASISCSFAGQRDSKLRLL

SEQ ID NO: 122 - protein, 33 aa, Lophius piscatorius
MQCIRCPAILALLALVLCGPSVSSQLDREQSDN

SEQ ID NO: 123 - protein, 32 aa, Lophius piscatorius
MGFLKFSPFLVVSILLLYQACGLQAVPLRSTL

SEQ ID NO: 124 - protein, 31 aa, Lophius piscatorius
MKRIHSLAGILLVLGLIQSSCRVLMQEADPS